SEQUENCE LISTING (3 sequences)

SEQ ID NO: 1 - protein, 108 aa, Homo sapiens
HPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 2 - protein, 134 aa, Homo sapiens
MDPQTAPSRALLLLLFLHLAFLGGRSHPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 3 - protein, 12 aa, Homo sapiens
AGTADCFWKYCV

(19) United States
(12) Patent Application Publication
(10) Pub. No.: US 2004/0253637 A1
(43) Pub. Date: Dec. 16, 2004

(54) Markers for differential diagnosis and methods of use thereof

(75) Inventors: Kenneth F. Buechler, Rancho Santa Fe, CA (US); Alan Maisel, Del Mar, CA (US); Joseph Michael Anderberg, Encinitas, CA (US); Paul H. McPherson, Encinitas, CA (US); Jeffrey R. Dahlen, San Diego, CA (US); Howard J. Kirchick, San Diego, CA (US)

Correspondence Address: FOLEY & LARDNER, P.O. BOX 80278, SAN DIEGO, CA, 92138-0278, US

(73) Assignee: Biosite Incorporated

(21) Appl. No.: 10/603,891

(22) Filed: Jun. 24, 2003

Related U.S. Application Data
(63) Continuation-in-part of application No. 10/410,572, filed on Apr. 8, 2003.
(63) Continuation-in-part of application No. 10/389,720, filed on Mar. 13, 2003.
Application No. 10/389,720 is a division of application No. 09/835,298, filed on Apr. 13, 2001.
(63) Continuation-in-part of application No. 10/371,149, filed on Feb. 20, 2003.
Application No. 10/371,149 is a continuation-in-part of application No. 10/225,082, filed on Aug. 20, 2002.
(63) Continuation-in-part of application No. 10/331,127, filed on Dec. 27, 2002.
(63) Continuation-in-part of application No. 10/330,696, filed on Dec. 27, 2002.
(63) Continuation-in-part of application No. PCT/US02/26604, filed on Aug. 20, 2002.
(63) Continuation-in-part of application No. PCT/US02/14219, filed on May 4, 2002.
(63) Continuation-in-part of application No. 10/139,086, filed on May 4, 2002.
(63) Continuation-in-part of application No. PCT/US02/11411, filed on Apr. 10, 2002.
Application No. PCT/US02/11411 is a continuation of application No. 09/835,298, filed on Apr. 13, 2001.
(60) Provisional application No. 60/436,392, filed on Dec. 24, 2002.
(60) Provisional application No. 60/313,775, filed on Aug. 20, 2001.
(60) Provisional application No. 60/334,964, filed on Nov. 30, 2001.
(60) Provisional application No. 60/346,485, filed on Jan. 2, 2002.
(60) Provisional application No. 60/436,392, filed on Dec. 24, 2002.
(60) Provisional application No. 60/436,301, filed on Dec. 24, 2002.
(60) Provisional application No. 60/313,775, filed on Aug. 20, 2001.
(60) Provisional application No. 60/334,964, filed on Nov. 30, 2001.
(60) Provisional application No. 60/346,485, filed on Jan. 2, 2002.
(60) Provisional application No. 60/288,871, filed on May 4, 2001.
(60) Provisional application No. 60/315,642, filed on Aug. 28, 2001.
(60) Provisional application No. 60/288,871, filed on May 4, 2001.
(60) Provisional application No. 60/315,642, filed on Aug. 28, 2001.

(51) Int. Cl. G01N 33/53

(52) U.S. Cl. 435/7.1; 436/518

(57) ABSTRACT

The present invention provides methods for the identification and use of diagnostic markers for differential diagnosis of diseases. In various aspects, the invention relates to methods and compositions able to determine the presence or absence of one, and preferably a plurality, of diseases that exhibit one or more similar or identical symptoms. Such methods and compositions can be used to provide assays and assay devices for use in determining the disease underlying one or more non-specific symptoms exhibited in a clinical setting.

This application is a continuation in part of U.S. patent application Ser. No. 10/330,696 filed Dec. 27, 2002, entitled “Markers for Differential Diagnosis and Methods of Use Thereof,” which claims priority to U.S. Provisional Patent Application No. 60/436,301 filed Dec. 24, 2002; and a continuation in part of U.S. patent application Ser. No. 10/139,086 filed May 4, 2002, entitled “Diagnostic Markers of Acute Coronary Syndromes and Methods of Use Thereof,” which claims priority to U.S. Provisional Patent Application No. 60/315,642 filed Aug. 28, 2001; and a continuation in part of International application PCT/US02/14219 filed May 4, 2002 which claims priority to U.S. Provisional Patent Application No. 60/288,871 filed May 4, 2001 and U.S. Provisional Patent Application No. 60/315,642 filed Aug. 28, 2001; and a continuation in part of U.S. patent application Ser. No. 10/389,720 filed Mar. 13, 2003, entitled “Use of B-type Natriuretic Peptide as a Prognostic Indicator in Acute Coronary Syndromes” which is a divisional of U.S. patent application Ser. No. 09/835,298 filed Apr. 13, 2001; and a continuation in part of International application PCT/JUS02/11441 filed Apr. 11, 2002 which claims priority to U.S. patent application Ser. No. 09/835,298 filed Apr. 13, 2001; and a continuation in part of U.S. patent application Ser. No. 10/371,149 filed Feb. 20, 2003 entitled “Diagnostic Markers of Stroke and Cerebral Injury and Methods of Use Thereof,” which is a continuation in part of Ser. No. 10/225,082 filed Aug. 20, 2002, which claims priority to U.S. Provisional Patent Application No. 60/313,775 filed Aug. 20, 2001; and a continuation in part of International application PCT/US02/26604 filed Aug. 20, 2002 which claims priority to U.S. Provisional Patent Application No. 60/313,775 filed Aug. 20, 2001, U.S. Provisional Patent Application No. 60/346,485 filed Jan. 2, 2002, and to U.S. Provisional Patent Application No. 60/334,964 filed Nov. 30, 2001; and a continuation in part of U.S. patent application Ser. No. 10/331,127 filed Dec. 27, 2002 and Ser. No. 10/410,572 filed Apr. 8, 2003, both of which claim priority to U.S. Provisional Patent Application No. 60/436,392 filed Dec. 24, 2002; a from each of which priority is claimed, and each of which is hereby incorporated by reference in its entirety, including all tables, figures, and claims.

### FIELD OF THE INVENTION

The present invention relates to the identification and use of diagnostic markers for differential diagnosis of diseases. In a various aspects, the invention relates to methods and compositions able to determine the presence or absence of one, and preferably a plurality, of diseases that exhibit one or more similar or identical symptoms.

### BACKGROUND OF THE INVENTION

The following discussion of the background of the invention is merely provided to aid the reader in understanding the invention and is not admitted to describe or constitute prior art to the present invention.

The clinical presentation of certain diseases can often be strikingly similar, even though the underlying diseases, and the appropriate treatments to be given to one suffering from the various diseases, can be completely distinct. For example, subjects may present in an urgent care facility exhibiting a deceptively simple constellation of apparent symptoms (e.g., fever, shortness of breath, dizziness, headache) that may be characteristic of a variety of unrelated conditions. Differential diagnosis methods involve the comparison of symptoms and/or diagnostic test results known to be associated with one or more diseases that exhibit a similar clinical presentation to the symptoms and/or diagnostic results exhibited by the subject, in order to identify the underlying disease or condition present in the subject.

Taking shortness of breath (referred to clinically as “dyspnea”) as an example, patients often present in a clinical setting with this symptom as the initial clinical presentation. This symptom considered in isolation may be indicative of conditions as diverse as asthma, chronic obstructive pulmonary disease (“COPD”), tracheal stenosis, obstructive endobroncheal tumor, pulmonary fibrosis, pneumoconiosis, lymphangitic carcinomatosis, kyphoscoliosis, pleural effusion, amyotrophic lateral sclerosis, congestive heart failure, coronary artery disease, myocardial infarction, cardiomyopathy, valvular dysfunction, left ventricle hypertrophy, pericarditis, arrhythmia, pulmonary embolism, metabolic acidosis, chronic bronchitis, pneumonia, anxiety, sepsis, aneurismic dissection, etc. See, e.g., Kelley's Textbook of Internal Medicine, 4^{th} Ed., Lippincott Williams & Wilkins, Philadelphia, Pa., 2000, pp. 2349-2354, “Approach to the Patient With Dyspnea”; Mulrow et al., J. Gen. Int. Med. 8: 383-92 (1993).

Differential diagnosis in the case of dyspnea involves identifying the particular condition causing shortness of breath in a given subject from amongst numerous possible causes. These methods often require that the clinician integrate information obtained from a battery of tests, leading to a clinical diagnosis that most closely represents the range of symptoms and/or diagnostic test results obtained for the subject. The tests required may include radiography, electrocardiogram, exercise treadmill testing, blood chemistry analysis, echocardiography, bronchoprovocation testing, spirometry, pulse oximetry, esophageal pH monitoring, laryngoscopy, computed tomography, histology, cytology, magnetic resonance imaging, etc. See, e.g., Morgan and Hodge, Am. Fam. Physician 57: 711-16 (1998). Because of the variety of tests that may need to be performed, obtaining sufficient information to arrive at a diagnosis can take hours or even days.

Differential diagnosis of chest pain requires the clinician to consider many possible causes, including differentiating between respiratory pain and pain associated with angina, or myocardial infarction and pleuritic and chest wall pain.

Differential diagnosis of diastolic and systolic dysfunction in patients suffering from heart failure is important since the therapies for each dysfunction are different. Further differentiation of atrial fibrillation from heart failure is critical for appropriate therapy.

In the area of infection, diffential diagnosis of viral versus bacterial is critical to the clinician delivering the appropriate therapy.

The acuteness or severity of the symptoms often dictates how rapidly a diagnosis must be established and treatment initiated. Immediate diagnosis and care of a patient experiencing a variety of acute conditions associated with dyspnea and chest pain can be critical. See, e.g., Harris, Aust. Fam. Physician 31: 802-06 (2002) (asthma); Goldhaber, Eur. Respir. J. Suppl. 35: 22s-27s (2002) (pulmonary embolism); Lundergan et al., Am. Heart J. 144: 456-62 (2002) (myocardial infarction). However, even in cases where the apparent symptoms appear relatively stable, rapid diagnosis, and the rapid initiation of treatment, can provide both relief from immediate discomfort and advantageous improvement in prognosis.

Each reference cited in the preceeding section is hereby incorporated by reference in its entirety, including all tables, figures, and claims.

### SUMMARY OF THE INVENTION

The present invention relates to the identification and use of diagnostic markers for differential diagnosis of diseases. The methods and compositions described herein can meet the need in the art for rapid, sensitive and specific diagnostic assays to be used in the diagnosis and differentiation of various diseases that are related in terms of one or more clinical characteristics.

In various aspects, the invention relates to materials and procedures for identifying the underlying cause of one or more symptoms that, when considered in isolation, may be related to a plurality of possible underlying diseases or conditions; to using such markers in diagnosing and treating a patient and/or to monitor the course of a treatment regimen; to using such markers to identify subjects at risk for one or more adverse outcomes an underlying disease or condition; and for screening compounds and pharmaceutical compositions that might provide a benefit in treating or preventing such diseases or conditions.

In a first aspect, the invention discloses methods for determining the presence or absence of a disease in a subject that is exhibiting a perceptible change in one or more physical characteristics (that is, one or more “symptoms”) that are indicative of a plurality of possible etiologies underlying the observed symptom(s). These methods comprise analyzing a test sample obtained from the subject for the presence or amount of one or more markers for one or more of the possible etiologies of the observed symptom(s). The presence or amount of such marker(s) in a sample obtained from the subject can be used to rule in or rule out one or more of the possible etiologies, thereby either providing a diagnosis (rule-in) and/or excluding one or more diagnoses (rule-out).

In certain embodiments, these markers can be used to rule in or rule out one or more possible etiologies of shortness of breath, or “dyspnea.” While the present invention is described hereinafter generally in terms of the differential diagnosis of diseases related to dyspnea, the skilled artisan will understand that the concepts of symptom-based differential diagnosis described herein are generally applicable to any physical characteristics that are indicative of a plurality of possible etiologies such as fever, chest pain (or “angina”), abdominal pain, neurologic dysfunction, disturbances in metabolic state, such as aberrant water, electrolyte, mineral, or acid-base metabolism, hypertension, dizziness, headache, etc.

In preferred embodiments, the present invention relates to methods in which a test sample is analyzed for the presence or amount of a plurality of markers related to a plurality of possible etiologies, so that the method is adapted to rule in or out a plurality of possible underlying causes based upon the analysis of a single sample. In the case of dyspnea, the plurality of markers are preferably selected to rule in or out a plurality of the following: asthma, atrial fibrillation, chronic obstructive pulmonary disease (“COPD”), tracheal stenosis, obstructive endobroncheal tumor, pulmonary fibrosis, pneumoconiosis, lymphangitic carcinomatosis, kyphoscoliosis, pleural effusion, amyotrophic lateral sclerosis, congestive heart failure, coronary artery disease, myocardial infarction, cardiomyopathy, valvular dysfunction, left ventricle hypertrophy, pericarditis, arrhythmia, pulmonary embolism, metabolic acidosis, chronic bronchitis, pneumonia, anxiety, sepsis, or aneurismic dissection. In a particularly preferred embodiment, the methods relate to defining the cause of dyspnea to rule in or rule out myocardial ischemia and cardiac necrosis, heart failure and pulmonary embolism. In yet another particularly preferred embodiment, the methods relate to defining the cause of dyspnea to rule in or rule out myocardial ischemia and cardiac necrosis, heart failure, pulmonary embolism and atrial fibrillation.

In the case of abdominal pain, the plurality of markers are preferably selected to rule in or out a plurality of the following: aortic aneurysm, mesenteric embolism, pancreatitis, appendicitis, myocardial infarction, one or more infectious diseases described above, influenza, esophageal carcinoma, gastric adenocarcinoma, colorectal adenocarcinoma, pancreatic tumors including ductal adenocarcinoma, cystadenocarcinoma, and insulinoma.

In the case of disturbanes of metabolic state, the plurality of markers are preferably selected to rule in or out a plurality of the following: diabetes mellitus, diabetic ketoacidosis, alcoholic ketoacidosis, respiratory acidosis, respiratory alkalosis, nonketogenic hyperglycemia, hypoglycemia, renal failure, interstitial renal disease, COPD, pneumonia, pulmonary and edema, asthma.

In certain embodiments, etiologies other than a disease that is to be ruled in or out, but which present similar symptoms, are referred to herein as “mimics” of a disease. For example, marker(s) able to differentiate one or more types of stroke from diseases that present similar symptoms, but that are not stroke (“stroke mimics”), are referred to herein as “stroke differential diagnostic markers.” The presence or amount of such marker(s) in a sample obtained from the subject can be used to rule in or rule out one or more of the following: stroke, thrombotic stroke, embolic stroke, lacunar stroke, hypoperfusion, intracerebral hemorrhage, and subarachnoid hemorrhage, thereby either providing a diagnosis (rule-in) and/or excluding a diagnosis (rule-out). Similarly, marker(s) able to differentiate congestive heart failure from diseases that present similar symptoms, but that are not congestive heart failure (“CHF mimics”), are referred to herein as “CHF differential diagnostic markers;” marker(s) able to differentiate myocardial infarction from diseases that present similar symptoms, but that are not myocardial infarction (“MI mimics”), are referred to herein as “MI differential diagnostic markers.” In another aspect, the present invention relates to methods and compositions for further subdividing congestive heart failure by distinguishing between systolic heart failure and diastolic heart failure. These methods comprise analyzing a test sample obtained from the subject for the presence or amount of one or more markers, the presence or amount of which can be used to rule in or out systolic heart failure and/or diastolic heart failure, or that can be used to distinguish between these two causes of congestive heart failure. Additional examples will be apparent to the skilled artisan from the description provided herein.

In another aspect, the present invention relates to methods and compositions for the differential diagnosis of atrial fibrillation and heart failure. The methods comprise analyzing a test sample obtained from the subject for the presence or amount of one or more markers, the presence or amount of which can be used to rule in or out heart failure or atrial fibrillation. In another aspect of this embodiment, the methods can be used to distinguish between systolic and diastolic dysfunction and atrial fibrillation. In yet another aspect of this embodiment, the methods can be used to distinguish between systolic and diastolic dysfunction, atrial fibrillation, myocardial ischemia and cardiac necrosis.

In yet another aspect, the present invention relates to methods and compositions for the differential diagnosis of aortic dissection and myocardial ischemia and necrosis. The methods comprise analyzing a test sample obtained from the subject for the presence or amount of one or more markers, the presence or amount of which can be used to rule in or out aortic dissection and myocardial ischemia and cardiac necrosis. In another aspect of this embodiment, the methods can be used to distinguish between aortic dissection, myocardial ischemia and cardiac necrosis and heart failure. In another aspect of this embodiment, the methods can be used to distinguish between aortic dissection, myocardial ischemia and cardiac necrosis, heart failure and atrial fibrillation.

Preferred markers of the invention can differentiate between myocardial infarction, congestive heart failure, and pulmonary embolism as a cause of dyspnea. Particularly preferred markers for these diseases are cardiac-specific troponin isoforms, B-type natriuretic peptide, and D-dimer, respectively. Each of these preferred markers are described in detail hereinafter.

The markers described herein may be used individually, but are preferably used as members of a marker “panel” comprising a plurality of markers that are measured in a sample. Such a panel may be analyzed in a number of fashions well known to those of skill in the art. For example, each member of a panel may be compared to a “normal” value, or a value identified as being indicative of the presence or absence of a particular disease. A particular diagnosis may depend upon the comparison of each marker to this value; alternatively, if only a subset of markers are outside of a normal range, this subset may be indicative of a particular diagnosis. In preferred embodiments, markers and marker panels are selected to exhibit at least 80% sensitivity, more preferably at least 90% sensitivity, and even more preferably at least 95% sensitivity, combined with at least 80% specificity, more preferably at least 90% specificity, and even more preferably at least 95% specificity. In particularly preferred embodiments, both the sensitivity and specificity are at least 85%, more preferably at least 90%, and even more preferably at least 95%.

Preferred marker panels of the present invention may be correlated to the presence or absence of a plurality of cardiovascular disorders. These marker panels preferably comprise one or more markers related to blood pressure regulation, and one or more markers related to cardiovascular damage. Additional markers may be added to such a panel, including preferably one or more markers related to inflammation and/or one or more markers related to coagulation and hemostasis. Suitable markers for inclusion in such panels are described in detail hereinafter.

Particularly preferred marker panels comprise, for example, one or more first marker(s) selected from the group consisting of atrial natriuretic factor, B-type natriuretic peptide or a marker related to B-type natriuretic peptide, C-type natriuretic factor, urotensin II, arginine vasopressin, aldosterone, angiotensin I, angiotensin II, angiotensin III, bradykinin, calcitonin, procalcitonin, calcitonin gene related peptide, adrenomedullin, calcyphosine, endothelin-2, endothelin-3, rennin, ANP, and urodilatin (referred to collectively as “markers related to blood pressure regulation”); and one or more second markers selected from the group consisting of free cardiac troponin I, free cardiac troponin T, cardiac troponin I in a complex comprising one or both of troponin T and troponin C, cardiac troponin T in a complex comprising one or both of troponin I and troponin C, free and complexed cardiac troponin I, free and complexed cardiac troponin T, creatine kinase-MB, myoglobin, glycogen phosphorylase-BB, annexin B, β-enolase, heart-type fatty acid binding protein, and S-100ao (referred to collectively as “markers related to myocardial injury”). Additional markers related to blood pressure regulation and myocardial injury are described hereinafter.

Additionally, one or more markers selected from the group consisting of C-reactive protein, interleukins, interleukin-1 receptor agonist, CD54, CD106, monocyte chemotactic protein-1, caspase-3, lipocalin-type prostaglandin D synthase, mast cell tryptase, eosinophil cationic protein, KL-6, haptoglobin, tumor necrosis factor a, tumor necrosis factor β, fibronectin, and vascular endothelial growth factor (“VEGF”) (referred to collectively as “markers related to inflammation”) may be included, as well as markers selected from the group consisting of plasmin, fibrinogen, D-dimer, β-thromboglobulin, platelet factor 4, fibrinopeptide A, platelet-derived growth factor, prothrombin fragment 1+2, plasmin-α2-antiplasmin complex, thrombin-antithrombin III complex, P-selectin, thrombin, von Willebrand factor, tissue factor, and thrombus precursor protein (referred to collectively as “markers related to coagulation and hemostasis”). Additional markers related to inflammation and markers related to coagulation and hemostasis are described hereinafter.

Additional markers and/or marker classes may be added to such panels to provide further ability to discriminate amongst diseases. For example, one or more markers selected from the group consisting of α-2 actin, basic calponin 1, β-1 integrin, acidic calponin, caldesmon, cysteine rich protein-2 (“CRP 2” or “CSRP 2”), elastin, fibrillin 1, latent transforming growth factor beta binding protein 4 (“LTBP 4”), smooth muscle myosin, smooth muscle myosin heavy chain, and transgelin (referred to collectively as “markers related to vascular tissue”) may be included in such a panel. Vascular tissue markers can in various embodiments be used as markers of aortic dissection and/or peripheral vascular disease or damage. Additional markers and marker classes are described hereinafter.

These markers may be combined in various combinations. For example, preferred panels may include B-type natriuretic peptide or a marker related to B-type natriuretic peptide, creatine kinase-MB, total cardiac troponin I, and myoglobin; B-type natriuretic peptide or a marker related to B-type natriuretic peptide, creatine kinase-MB, total cardiac troponin I, myoglobin, and C-reactive protein; B-type natriuretic peptide or a marker related to B-type natriuretic peptide, D-dimer, creatine kinase-MB, total cardiac troponin I, and myoglobin; and/or B-type natriuretic peptide or a marker related to B-type natriuretic peptide, D-dimer, creatine kinase-MB, total cardiac troponin I, myoglobin, and C-reactive protein. Such panels may distinguish a plurality of cardiovascular disorders selected from the group consisting of myocardial infarction, congestive heart failure, acute coronary syndrome, unstable angina, and pulmonary embolism.

Likewise, a panel may comprise a plurality of markers selected to diagnose and/or distinguish amongst a plurality of cerebrovascular disorders. Preferred marker panels of the present invention comprise one or more markers related to blood pressure regulation, and one or more markers related to neural tissue injury. Additional markers may be added to such a panel, including preferably one or more markers related to inflammation, and/or one or more markers related to apoptosis, and/or one or more acute phase markers and/or one or more markers related to coagulation and hemostasis.

Exemplary markers related to blood pressure regulation, to inflammation, and to coagulation and hemostasis are described above. One or more markers related to neural tissue injury include those selected from the group consisting of precerebellin 1, cerebillin 1., cerebellin 3, chimerin 1, chimerin 2, calbrain, calbindin D, brain tubulin, brain fatty acid binding protein (“B-FABP”), brain derived neurotrophic factor (“BDNF”), carbonic anhydrase XI, CACNA1A calcium channel gene, nerve growth factor β, atrophin 1, apolipoprotein E4-1, protein 4.1B, 14-3-3 protein, ciliary neurotrophic factor, creatine kinase-BB, C-tau, glial fibrillary acidic protein (“GFAP”), neural cell adhesion molecule (“NCAM”), neuron specific enolase, S-100b, prostaglandin D synthase, neurokinin A, neurotensin, and secretagogin. Additional exemplary markers related to neural tissue injury are described hereinafter. One or more markers related to apoptosis include those selected from the group consisting of spectrin, cathepsin D, caspase 3, s-acetyl glutathione, and ubiquitin fusion degradation protein 1 homolog. Suitable additional markers for inclusion in such panels are described in detail hereinafter. The presence or amount of the markers in such panels may be correlated to the presence or absence of a plurality of cerebrovascular disorders.

Additionally, one or more acute phase reactants include those selected from the group consisting of hepcidin, HSP-60, HSP-65, HSP-70, S-FAS ligand, asymmetric dimethylarginine (an endogenous inhibitor of nitric oxide synthase), matrix metalloproteins 11, 3, and 9, defensin HBD 1, defensin HBD 2, serum amyloid A, oxidized LDL, insulin like growth factor, transforming growth factor β, e-selectin, glutathione-S-transferase, hypoxia-inducible factor-1α, inducible nitric oxide synthase (“I-NOS”), intracellular adhesion molecule, lactate dehydrogenase, monocyte chemoattractant peptide-1 (“MCP-1”), n-acetyl aspartate, prostaglandin E2, receptor activator of nuclear factor (“RANK”) ligand, TNF receptor superfamily member 1A, TNFα, vascular cell adhesion molecule, and cystatin C.

An exemplary marker panel selected to diagnose and/or distinguish amongst a plurality of cerebrovascular disorders includes a plurality (and preferably each) of B-type natriuretic peptide or a marker related to B-type natriuretic peptide, caspase-3, interleukin-8, creatine kinase-BB, C-reactive protein, S-100b, matrix metalloprotein-9, and neural cell adhesion molecule. The presence or amount of the markers in such panels may be correlated to the presence or absence of a plurality of cerebrovascular disorders. Additional markers and marker panels are described hereinafter.

The presence or amount of the markers in such panels may be correlated to the presence or absence of a plurality of cerebrovascular disorders. Additional markers are described hereinafter. As described hereinafter, the markers described herein may be indicative of a plurality of diseases, depending on the status of other markers in a panel. For example, certain markers are generally elevated in inflammation resulting from a variety of causes. Thus, alone, a single marker may not be diagnostic per se, but as part of a panel, the marker can provide important diagnostic and/or prognostic information.

In a related aspect, the presence or amount of markers that are selected to diagnose and/or distinguish amongst a plurality of cerebrovascular disorders may also be used prognostically, in order to identify patients at risk for a future onset of a cerebrovascular disorder. Such uses may find particular interest in monitoring patients known to be at increased risk for such onset. For example, patients undergoing carotid endarterectomy are known to be at risk for cerebral ischemia. Outcomes of such ischemia include intraoperative and perioperative stroke, neurologic deficit, and death. Cerebral ischemia is also a risk of procedures such as hypothermic circulatory arrest, aortic valve replacement, mitral valve replacement, coronary artery surgery, endograft repair of aortic aneurism, coronary artery bypass graft surgery, laryngeal mask insertion, and repair of congenital heart defects. Thus, the present invention also relates to methods and compositions for monitoring the status of patients undergoing such procedures to identify at-risk patients.

In another aspect, the present invention relates to methods for identifying marker panels for use in the foregoing methods. The sensitivity and specificity of a diagnostic test depends on more than just the “quality” of the test—they also depend on the definition of what constitutes an abnormal test. In practice, Receiver Operating Characteristic curves, or “ROC” curves, are typically calculated by plotting the value of a variable versus its relative frequency in “normal” and “disease” populations. For any particular marker, a distribution of marker levels for subjects with and without a disease will likely overlap. Under such conditions, a test does not absolutely distinguish normal from disease with 100% accuracy, and the area of overlap indicates where the test cannot distinguish normal from disease. A threshold is selected, above which (or below which, depending on how a marker moves with the disease) the test is considered to be abnormal and below which the test is considered to be normal. The area under the ROC curve is a measure of the probability that the perceived measurement will allow correct identification of a condition. ROC curves can be used even when test results don't necessarily give an accurate numeric value for a marker level; that is, as long as one can rank results, one can create an appropriate ROC curve. Such methods are well known in the art. See, e.g., Hanley et al., Radiology 143: 29-36 (1982).

In preferred embodiments, particular thresholds for one or more markers in a panel are not relied upon to determine if a profile of marker levels obtained from a subject are indicative of a particular diagnosis. Rather, the present invention may utilize an evaluation of the entire profile of markers. By plotting ROC curves for the sensitivity of a particular panel of markers versus 1-(specificity) for the panel at various cutoffs, a profile of marker measurements from a subject may be considered together to provide a global probability (expressed either as a numeric score or as a percentage risk) that the symptom(s) observed in an individual are caused by a partiuclar underlying disease. In such embodiments, an increase in a certain subset of markers may be sufficient to indicate a particular diagnosis in one patient, while an increase in a different subset of markers may be sufficient to indicate the same or a different diagnosis in another patient.

One or more markers may lack predictive value when considered alone, but when used as part of a panel, such markers may be of great value in determining a particular diagnosis. Weighting factors may also be applied to one or more markers in a panel, for example, when a marker is of particularly high utility in identifying a particular diagnosis. While the exemplary panels described herein can provide the ability to determine a diagnosis underlying, e.g., dyspnea, one or more markers may be replaced, added, or subtracted from these exemplary panels while still providing clinically useful results.

In yet other embodiments, multiple determinations of one or more markers can be made, and a temporal change in the markers can be used to rule in or out one or more particular etiologies for observed symptom(s). For example, one or more markers may be determined at an initial time, and again at a second time, and the change (or lack thereof) in the marker level(s) over time determined. In such embodiments, an increase in the marker from the initial time to the second time may be diagnostic of a particular disease underlying one or more symptoms. Likewise, a decrease in the marker from the initial time to the second time may be indicative of a particular disease underlying one or more symptoms. Temporal changes in one or more markers may also be used together with single time point marker levels to increase the discriminating power of marker panels.

In yet another embodiment, multiple determinations of one or more diagnostic or prognostic markers can be made, and a temporal change in the marker can be used to monitor the efficacy appropriate therapies. In such an embodiment, one might expect to see a decrease or an increase in the marker(s) over time during the course of effective therapy.

The skilled artisan will understand that, while in certain embodiments comparative measurements are made of the same diagnostic marker at multiple time points, one could also measure a given marker at one time point, and a second marker at a second time point, and a comparison of these markers may provide diagnostic information. Similarly, the skilled artisan will understand that serial measurements and changes in markers or the combined result over time may also be of diagnostic and/or prognostic value.

The skilled artisan will understand that associating one or more diagnostic markers with the presence or absence of a particular disease is a statistical analysis. For example, the presence or absence of a particular marker level may signal that a patient is more likely to suffer from a disease, as determined by a level of statistical significance. Statistical significance is often determined by comparing two or more populations, and determining a confidence interval and/or a p value. See, e.g., Dowdy and Wearden, Statistics for Research, John Wiley & Sons, New York, 1983. Preferred confidence intervals of the invention are 90%, 95%, 97.5%, 98%, 99%, 99.5%, 99.9% and 99.99%, while preferred p values are 0.1, 0.05, 0.025, 0.02, 0.01, 0.005, 0.001, and 0.0001.

In yet another aspect, the invention relates to methods for determining a treatment regimen for use in a subject. The methods preferably comprise identifying the underlying cause of one or more symptoms that, when considered in isolation, may be related to a plurality of possible underlying diseases or conditions as described herein. One or more treatment regimens can then be selected to treat the underlying disease in the subject.

In a related aspect, the invention relates to methods for determining a treatment regimen for use in a subject suffering from congestive heart failure. The methods preferably comprise performing the methods described herein to rule in or out systolic heart failure and/or diastolic heart failure, or to distinguish between these two causes of congestive heart failure. One or more treatment regimens can then be selected to treat the type of congestive heart failure in the subject.

In a further aspect, the invention relates to kits for identifying the underlying cause of one or more symptoms that, when considered in isolation, may be related to a plurality of possible underlying diseases or conditions. These kits preferably comprise devices and reagents for measuring one or more marker levels in a patient sample, and instructions for performing the assay. Optionally, the kits may contain one or more means for correlating marker level(s) in order to rule in or rule out one or more potential etiologies of the observed symptoms. Such kits preferably contain sufficient reagents to perform one or more such determinations, and/or Food and Drug Administration (FDA)-approved labeling.

In a related aspect, the invention relates to kits to rule in or out systolic heart failure and/or diastolic heart failure, or to distinguish between these two causes of congestive heart failure. These kits preferably comprise devices and reagents for measuring one or more marker levels in a patient sample, and instructions for performing the assay. Optionally, the kits may contain one or more means for correlating marker level(s) in order to distinguish between systolic heart failure and diastolic heart failure. Such kits preferably contain sufficient reagents to perform one or more such determinations, and/or Food and Drug Administration (FDA)-approved labeling.

In yet a further aspect, the invention relates to devices for identifying the underlying cause of one or more symptoms that, when considered in isolation, may be related to a plurality of possible underlying diseases or conditions. Such devices preferably contain a plurality of discrete, independently addressable locations, or “diagnostic zones,” each of which is related to a particular marker of interest. Following reaction of a sample with the devices, a signal is generated from the diagnostic zone(s), which may then be correlated to the presence or amount of the markers of interest. Such markers may then be used to rule in or out one or more potential etiologies of the observed symptoms.

In a related aspect, the invention relates to devices to rule in or out systolic heart failure and/or diastolic heart failure, or to distinguish between these two causes of congestive heart failure. Such devices preferably contain a plurality of diagnostic zones, each of which is related to a particular marker of interest. Following reaction of a sample with the devices, a signal is generated from the diagnostic zone(s), which may then be correlated to the presence or amount of the markers of interest. Such markers may then be used to distinguish between systolic heart failure and diastolic heart failure.

### DETAILED DESCRIPTION OF THE INVENTION

The present invention relates to methods and compositions for symptom-based differential diagnosis of diseases in subjects.

Patients presenting for medical treatment often exhibit one or a few primary observable changes in bodily characteristics or functions that are indicative of disease. Often, these “symptoms” are nonspecific, in that a number of potential diseases can present the same observable symptom or symptoms. A typical list of nonspecific symptoms might include one or more of the following: shortness of breath (or dyspnea), chest pain, fever, dizziness, and headache. These symptoms can be quite common, and the number of diseases that must be considered by the clinician can be astoundingly broad.

Taking shortness of breath (referred to clinically as “dyspnea”) as an example, this symptom considered in isolation may be indicative of conditions as diverse as asthma, chronic obstructive pulmonary disease (“COPD”), tracheal stenosis, obstructive endobroncheal tumor, pulmonary fibrosis, pneumoconiosis, lymphangitic carcinomatosis, kyphoscoliosis, pleural effusion, amyotrophic lateral sclerosis, congestive heart failure, coronary artery disease, myocardial infarction, atrial fibrillation, cardiomyopathy, valvular dysfunction, left ventricle hypertrophy, pericarditis, arrhythmia, pulmonary embolism, metabolic acidosis, chronic bronchitis, pneumonia, anxiety, sepsis, aneurismic dissection, etc. See, e.g., Kelley's Textbook of Internal Medicine, 4^{th} Ed., Lippincott Williams & Wilkins, Philadelphia, Pa., 2000, pp. 2349-2354, “Approach to the Patient With Dyspnea”; Mulrow et al., J. Gen. Int. Med. 8: 383-92 (1993).

Similarly, chest pain, when considered in isolation, may be indicative of stable angina, unstable angina, myocardial ischemia, atrial fibrillation, myocardial infarction, musculoskeletal injury, cholecystitis, gastroesophageal reflux, pulmonary embolism, pericarditis, aortic dissection, pneumonia, anxiety, etc. Moreover, the classification of chest pain as stable or unstable angina (or even mild myocardial infarction) in cases other than definitive myocardial infarction is completely subjective. The diagnosis, and in this case the distinction, is made not by angiography, which may quantify the degree of arterial occlusion, but rather by a physician's interpretation of clinical symptoms.

Differential diagnosis refers to methods for diagnosing the particular disease(s) underlying the symptoms in a particular subject, based on a comparison of the characteristic features observable from the subject to the characteristic features of those potential diseases. Depending on the breadth of diseases that must be considered in the differential diagnosis, the types and number of tests that must be ordered by a clinician can be quite large. In the case of dyspnea for example, the clinician may order tests from a group that includes radiography, electrocardiogram, exercise treadmill testing, blood chemistry analysis, echocardiography, bronchoprovocation testing, spirometry, pulse oximetry, esophageal pH monitoring, laryngoscopy, computed tomography, histology, cytology, magnetic resonance imaging, etc. See, e.g., Morgan and Hodge, Am. Fam. Physician 57: 711-16 (1998). The clinician must then integrate information obtained from a battery of tests, leading to a clinical diagnosis that most closely represents the range of symptoms and/or diagnostic test results obtained for the subject.

The present invention describes methods and compositions that can assist in the differential diagnosis of one or more nonspecific symptoms by providing diagnostic markers that are designed to rule in or out one, and preferably a plurality, of possible etiologies for the observed symptoms. The concept of symptom-based differential diagnosis described herein can provide panels of diagnostic markers designed be considered in concert to distinguish between possible diseases that underlie a nonspecific symptom observed in a patient.

Definitions

The term “fever” as used herein refers to a body temperature greater than 100° C. orally or 100.8° C. rectally. In the case of fever, a plurality of markers are preferably selected to rule in or out a plurality of the following: sepsis; arteritis; sarcoidosis; and one or more infectious diseases, including infection by Staphyloccus species, Nisseria species, Pneumococcal species, Listeria species, Anthrax, Nocardia species, Salmonella species, Shigella species, Haemophilus species, Brucella species, Vibrio species including V. cholerae, Franciscella tularensis, Yersinia pestis, Pseudomonas species, Clostridia species including C. tetani, C. perfringens, C. ramosum, C. botulinum, and C. septicum, Actinomyces species, Treponema pallidum, Borrelia species including B. burgdorferi, Leptospira species, Mycobacterium species including M. tuberculosis, M. bovis, M. leprae, and M. africanum, Histoplasma species, Escherichia coli, Coccidioides species, Blastomyces species, Paracoccidioides species, Sporothrix species, Cryptococcus species, Candida species, and Aspergillus species; Rickettsial diseases including Rocky Mountain spotted fever, Q fever, typhus, trench fever, and cat-scratch fever; parasitic diseases including Malaria, Babesiosis, African sleeping sickness, Trypanosomiasis, Leishmaniasis, Toxoplasmosis, and Amebiasis; viral infection by influenza virus, parainfluenza virus, mumps virus, adenovirus, respiratory syncytial virus, rhinovirus, poliovirus, coxackievirus, echovirus, rubeola virus, rubella virus, parvovirus, hepatitis A, B, C, D, or E, cytomegalovirus, Epstein-Barr virus, Herpes simplex virus, Varicella-zoster virus, Alphavirus, Flaviviruses including yellow fever virus, dengue fever virus, Japanese encephalitis virus, and St. Louis encephalitis virus, West Nile virus, Colorado tick fever virus, Rabies virus, Arenavirus, Marburg agent, and Ebola virus.

The term “cerebrovascular disorder” as used herein refers to vascular and parenchymal cerebral abnormalities. The term is intended to include ischemic stroke (e.g., atherogenic embolism, thrombotic occlusion, lacunar stroke, cardioembolytic stroke), hemorrhagic stroke, transient ischemic attack, subarachnoid hemorrhage, cerebral vasospasm, hypertensive small vessel disease, vascular inflammatory conditions, aneurysm, arteriovenous malformations, and cerebral amyloid angiopathy (a disorder in which deposition of amyloid within the arterial media and adventitia leads to intracerebral hemorrhage). This list is not meant to be limiting.

The term “cardiovascular disorder” as used herein refers to abnormalities of the heart and vasculature. The term is intended to include, but is not limited to, renovascular hypertension, congestive heart failure, aortic aneurysm, iliac or femoral aneurysm, pulmonary embolism, myocardial infarction, acute coronary syndrome, angina, primary hypertension, atrial fibrillation, systolic dysfunction, diastolic dysfunction, myocarditis, atherosclerosis, atrial tachycardia, ventricular fibrillation, endocarditis, and peripheral vascular disease.

The term “neurologic dysfunction” as used herein refers to a loss of one or more normal physiological or mental functions having a neurogenic etiology. The skilled artisan will understand that neurologic dysfunction is a common symptom in various systemic disorders (e.g., alcoholism, vascular disease, stroke, autoimmunity, metabolic disorders, aging, etc.). Specific neurologic dysfunctions include, but are not limited to, pain, headache, aphasia, apraxia, agnosia, amnesia, stupor, coma, delirium, dementia, seizure, migraine insomnia, hypersomnia, sleep apnea, tremor, dyskinesia, paralysis, etc.

The term “hypertension” as used herein refers to a systolic blood pressure of greater than or equal to 140 mm Hg and/or a diastolic blood pressure of greater than or equal to 90 mm Hg. Hypertension can include isolated systolic hypertension (i.e., no elevation in diastolic blood pressure). In the case of hypertension, the plurality of markers are preferably selected to rule in or out a plurality of the following: left ventricular failure, atherosclerosis, renal disease including chronic glomerulonephritis, and polycystic renal disease, coartation of the aorta, renal arteriall stenosis, and hyperparathyroidism.

The term “condition within the differential diagnosis of a symptom” as used herein refers to a pathologic state that is known to be causative of a particular perceptible change in one or more physical characteristics exhibited by a subject suffering from the pathologic state, as compared to a normal subject. The concept of differential diagnosis is well established to those of skill in the art. See, e.g., Beck, Tutorials in Differential Diagnosis, Churchill Livingstone, 2002; Zackon, Pulmonary Differential Diagnosis, Elsevier, 2000; Jamison, Differential Diagnosis for Primary Practice, Churchill Livingstone, 1999; Bouchier et al., French's Index of Differential Diagnosis, Oxford University Press, 1997.

The term “marker” as used herein refers to proteins, polypeptides, phospholipids, or small molecules to be used as targets for screening test samples obtained from subjects. “Proteins or polypeptides” used as markers in the present invention are contemplated to include any fragments thereof, in particular, immunologically detectable fragments.

The term “related marker” as used herein refers to one or more fragments of a particular marker or its biosynthetic parent that may be detected as a surrogate for the marker itself or as independent markers. For example, human BNP is derived by proteolysis of a 108 amino acid precursor molecule, referred to hereinafter as BNP₁₋₁₀₈. Mature BNP, or “the BNP natriuretic peptide,” or “BNP-32” is a 32 amino acid molecule representing amino acids 77-108 of this precursor, which may be referred to as BNP₇₇₋₁₀₈. The remaining residues 1-76 are referred to hereinafter as BNP₁₋₇₆.

The sequence of the 108 amino acid BNP precursor pro-BNP (BNP₁₋₁₀₈) is as follows, with mature BNP (BNP₇₇₋₁₀₈) underlined:

| | | |
|---|---|---|
| HPLGSPGSAS DLETSGLQEQ RNHLQGKLSE LQVEQTSLEP LQESPRPTGV | 50 | (SEQ ID NO: 1) |
| WKSREVATEG IRGHRKMVLY TLRAPRSPKM VQGSGCFGRK MDRISSSSGL | 100 | |
| GCKVLRRH. | 108 | |

BNP₁₋₁₀₈ is synthesized as a larger precursor pre-pro-BNP having the following sequence (with the “pre” sequence shown in bold):

| | | |
|---|---|---|
| MDPQTAPSRA LLLLLFLHLA FLGGRSHPLG SPGSASDLET SGLQEQRNHL | 50 | (SEQ ID NO: 2) |
| QGKLSELQVE QTSLEPLQES PRPTGVWKSR EVATEGIRGH RKMVLYTLRA | 100 | |
| PRSPKMVQGS GCFGRKMDRI SSSSGLGCKV LRRH. | 134 | |

While mature BNP itself may be used as a marker in the present invention, the prepro-BNP, BNP₁₋₁₀₈ and BNP₁₋₇₆ molecules represent BNP-related markers that may be measured either as surrogates for mature BNP or as markers in and of themselves. In addition, one or more fragments of these molecules, including BNP-related polypeptides selected from the group consisting of BNP₇₇₋₁₀₆, BNP₇₉₋₁₀₆, BNP₇₆₋₁₀₇, BNP₆₉₋₁₀₈, BNP₇₉₋₁₀₈, BNP₈₀₋₁₀₈, BNP₈₁₋₁₀₈, BNP₈₃₋₁₀₈, BNP₃₉₋₈₆, BNP₅₃₋₈₅, BNP₆₆₋₉₈, BNP₃₀₋₁₀₃, BNP₁₋₁₀₇, BNP₉₋₁₀₆, and BNP₃₋₁₀₈ may also be present in circulation. In addition, natriuretic peptide fragments, including BNP fragments, may comprise one or more oxidizable methionines, the oxidation of which to methionine sulfoxide or methionine sulfone produces additional BNP-related markers. See, e.g., U.S. patent Ser. No. 10/419,059, filed Apr. 17, 2003, which is hereby incorporated by reference in its entirety including all tables, figures and claims.

Because production of BNP-related markers is an ongoing process that may be a function of, inter alia, the elapsed time between onset of an event triggering natriuretic peptide release into the tissues and the time the sample is obtained or analyzed; the elapsed time between sample acquisition and the time the sample is analyzed; the type of tissue sample at issue; the storage conditions; the quantity of proteolytic enzymes present; etc., it may be necessary to consider this degradation when both designing an assay for one or more natriuretic peptides, and when performing such an assay, in order to provide an accurate prognostic or diagnostic result. In addition, individual antibodies that distinguish amongst a plurality of natriuretic peptide (e.g., BNP) fragments may be individually employed to separately detect the presence or amount of different fragments. The results of this individual detection may provide a more accurate prognostic or diagnostic result than detecting the plurality of fragments in a single assay. For example, different weighting factors may be applied to the various fragment measurements to provide a more accurate estimate of the amount of natriuretic peptide originally present in the sample.

In a similar fashion, many of the markers described herein are synthesized as larger precursor molecules, which are then processed to provide mature marker; and/or are present in circulation in the form of fragments of the marker. Thus, “related markers” to each of the markers described herein may be identified and used in an analogous fashion to that described above for BNP.

Preferably, the methods described hereinafter utilize one or more markers that are derived from the subject. The term “subject-derived marker” as used herein refers to protein, polypeptide, phospholipid, nucleic acid, prion, or small molecule markers that are expressed or produced by one or more cells of the subject. The presence, absence, or amount of one or more markers may indicate that a particular disease is present, or may indicate that a particular disease is absent. Additional markers may be used that are derived not from the subject, but rather that are expressed by pathogenic or infectious organisms that are correlated with a particular disease. Such markers are preferably protein, polypeptide, phospholipid, nucleic acid, prion, or small molecule markers that identify the infectious diseases described above.

The term “test sample” as used herein refers to a sample of bodily fluid obtained for the purpose of diagnosis, prognosis, or evaluation of a subject of interest, such as a patient. In certain embodiments, such a sample may be obtained for the purpose of determining the outcome of an ongoing condition or the effect of a treatment regimen on a condition. Preferred test samples include blood, serum, plasma, cerebrospinal fluid, urine, saliva, sputum, and pleural effusions. In addition, one of skill in the art would realize that some test samples would be more readily analyzed following a fractionation or purification procedure, for example, separation of whole blood into serum or plasma components.

As used herein, a “plurality” as used herein refers to at least two. Preferably, a plurality refers to at least 3, more preferably at least 5, even more preferably at least 10, even more preferably at least 15, and most preferably at least 20. In particularly preferred embodiments, a plurality is a large number, i.e., at least 100.

The term “subject” as used herein refers to a human or non-human organism. Thus, the methods and compositions described herein are applicable to both human and veterinary disease. Further, while a subject is preferably a living organism, the invention described herein may be used in post-mortem analysis as well. Preferred subjects are “patients,” i.e., living humans that are receiving medical care. This includes persons with no defined illness who are being investigated for signs of pathology.

The term “diagnosis” as used herein refers to methods by which the skilled artisan can estimate and/or determine whether or not a patient is suffering from a given disease or condition. The skilled artisan often makes a diagnosis on the basis of one or more diagnostic indicators, i.e., a marker, the presence, absence, or amount of which is indicative of the presence, severity, or absence of the condition.

Similarly, a prognosis is often determined by examining one or more “prognostic indicators.” These are markers, the presence or amount of which in a patient (or a sample obtained from the patient) signal a probability that a given course or outcome will occur. For example, when one or more prognostic indicators reach a sufficiently high level in samples obtained from such patients, the level may signal that the patient is at an increased probability for experiencing a future stroke in comparison to a similar patient exhibiting a lower marker level. A level or a change in level of a prognostic indicator, which in turn is associated with an increased probability of morbidity or death, is referred to as being “associated with an increased predisposition to an adverse outcome” in a patient. Preferred prognostic markers can predict the onset of delayed neurologic deficits in a patient after stroke, or the chance of future stroke.

The term “correlating,” as used herein in reference to the use of diagnostic and markers, refers to comparing the presence or amount of the marker(s) in a patient to its presence or amount in persons known to suffer from, or known to be at risk of, a given condition; or in persons known to be free of a given condition. As discussed above, a marker level in a patient sample can be compared to a level known to be associated with a specific diagnosis. The sample's marker level is said to have been correlated with a diagnosis; that is, the skilled artisan can use the marker level to determine whether the patient suffers from a specific type diagnosis, and respond accordingly. Alternatively, the sample's marker level can be compared to a marker level known to be associated with a good outcome (e.g., the absence of disease, etc.). In preferred embodiments, a profile of marker levels are correlated to a global probability or a particular outcome using ROC curves.

The phrase “determining the diagnosis” as used herein refers to methods by which the skilled artisan can determine the presence or absence of a particular disease in a patient. The term “diagnosis” does not refer to the ability to determine the presence or absence of a particular disease with 100% accuracy, or even that a given course or outcome is more likely to occur than not. Instead, the skilled artisan will understand that the term “diagnosis” refers to an increased probability that a certain disease is present in the subject. In preferred embodiments, a diagnosis indicates about a 5% increased chance that a disease is present, about a 10% chance, about a 15% chance, about a 20% chance, about a 25% chance, about a 30% chance, about a 40% chance, about a 50% chance, about a 60% chance, about a 75% chance, about a 90% chance, and about a 95% chance. The term “about” in this context refers to +/−2%.

The term “discrete” as used herein refers to areas of a surface that are non-contiguous. That is, two areas are discrete from one another if a border that is not part of either area completely surrounds each of the two areas.

The term “independently addressable” as used herein refers to discrete areas of a surface from which a specific signal may be obtained.

The term “antibody” as used herein refers to a peptide or polypeptide derived from, modeled after or substantially encoded by an immunoglobulin gene or immunoglobulin genes, or fragments thereof, capable of specifically binding an antigen or epitope. See, e.g. Fundamental Immunology, 3^{rd} Edition, W. E. Paul, ed., Raven Press, N.Y. (1993); Wilson (1994) J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

Identification of Marker Panels

In accordance with the present invention, there are provided methods and systems for the identification of one or more markers for the differential diagnosis of one or more nonspecific symptoms exhibited by a subject. Suitable methods for identifying markers useful for the diagnosis of disease states are described in detail in U.S. Provisional Patent Application No. 60/436,392 filed Dec. 24, 2002, entitled METHOD AND SYSTEM FOR DISEASE DETECTION USING MARKER COMBINATIONS, and U.S. patent application Ser. No. 10/331,127 filed Dec. 27, 2002, entitled METHOD AND SYSTEM FOR DISEASE DETECTION USING MARKER COMBINATIONS, each of which is hereby incorporated by reference in its entirety, including all tables, figures, and claims.

One skilled in the art will also recognize that univariate analysis of markers can be performed and the data from the univariate analyses of multiple markers can be combined to form panels of markers to differentiate different disease conditions. In forming panels of markers to define the cause of dypsnea, for example, markers related to each cause of dypsnea should be considered.

In developing a panel of markers useful in differential diagnosis, data for a number of potential markers may be obtained from a group of subjects by testing for the presence or level of certain markers. The group of subjects is divided into two sets. The first set includes subjects who have been confirmed as having a disease or, more generally, being in a first condition state. For example, this first set of patients may be those that have recently had a stroke. The confirmation of this condition state may be made through a more rigorous and/or expensive testing confirm the condition state. Hereinafter, subjects in this first set will be referred to as “diseased”.

The second set of subjects is simply those who do not fall within the first set. Subjects in this second set will hereinafter be referred to as “non-diseased”. Preferably, the first set and the second set each have an approximately equal number of subjects.

The data obtained from subjects in these sets includes levels of a plurality of markers. Preferably, data for the same set of markers is available for each patient. This set of markers may include all candidate markers that may be suspected as being relevant to the detection of a particular disease or condition. Actual known relevance is not required. Embodiments of the methods and systems described herein may be used to determine which of the candidate markers are most relevant to the diagnosis of the disease or condition. The levels of each marker in the two sets of subjects may be distributed across a broad range, e.g., as a Gaussian distribution. However, no distribution fit is required.

As noted above, a marker often is incapable of definitively identifying a patient as either diseased or non-diseased. For example, if a patient is measured as having a marker level that falls within the overlapping region, the results of the test will be useless in diagnosing the patient. An artificial cutoff may be used to distinguish between a positive and a negative test result for the detection of the disease or condition. Regardless of where the cutoff is selected, the effectiveness of the single marker as a diagnosis tool is unaffected. Changing the cutoff merely trades off between the number of false positives and the number of false negatives resulting from the use of the single marker. The effectiveness of a test having such an overlap is often expressed using a ROC (Receiver Operating Characteristic) curve. ROC curves are well known to those skilled in the art.

The horizontal axis of the ROC curve represents (1-specificity), which increases with the rate of false positives. The vertical axis of the curve represents sensitivity, which increases with the rate of true positives. Thus, for a particular cutoff selected, the value of (1-specificity) may be determined, and a corresponding sensitivity may be obtained. The area under the ROC curve is a measure of the probability that the measured marker level will allow correct identification of a disease or condition. Thus, the area under the ROC curve can be used to determine the effectiveness of the test.

As discussed above, the measurement of the level of a single marker may have limited usefulness. The measurement of additional markers provides additional information, but the difficulty lies in properly combining the levels of two potentially unrelated measurements. In the methods and systems according to embodiments of the present invention, data relating to levels of various markers for the sets of diseased and non-diseased patients may be used to develop a panel of markers to provide a useful panel response. The data may be provided in a database such as Microsoft Access, Oracle, other SQL databases or simply in a data file. The database or data file may contain, for example, a patient identifier such as a name or number, the levels of the various markers present, and whether the patient is diseased or non-diseased.

Next, an artificial cutoff region may be initially selected for each marker. The location of the cutoff region may initially be selected at any point, but the selection may affect the optimization process described below. In this regard, selection near a suspected optimal location may facilitate faster convergence of the optimizer. In a preferred method, the cutoff region is initially centered about the center of the overlap region of the two sets of patients. In one embodiment, the cutoff region may simply be a cutoff point. In other embodiments, the cutoff region may have a length of greater than zero. In this regard, the cutoff region may be defined by a center value and a magnitude of length. In practice, the initial selection of the limits of the cutoff region may be determined according to a pre-selected percentile of each set of subjects. For example, a point above which a pre-selected percentile of diseased patients are measured may be used as the right (upper) end of the cutoff range.

Each marker value for each patient may then be mapped to an indicator. The indicator is assigned one value below the cutoff region and another value above the cutoff region. For example, if a marker generally has a lower value for non-diseased patients and a higher value for diseased patients, a zero indicator will be assigned to a low value for a particular marker, indicating a potentially low likelihood of a positive diagnosis. In other embodiments, the indicator may be calculated based on a polynomial. The coefficients of the polynomial may be determined based on the distributions of the marker values among the diseased and non-diseased subjects.

The relative importance of the various markers may be indicated by a weighting factor. The weighting factor may initially be assigned as a coefficient for each marker. As with the cutoff region, the initial selection of the weighting factor may be selected at any acceptable value, but the selection may affect the optimization process. In this regard, selection near a suspected optimal location may facilitate faster convergence of the optimizer. In a preferred method, acceptable weighting coefficients may range between zero and one, and an initial weighting coefficient for each marker may be assigned as 0.5. In a preferred embodiment, the initial weighting coefficient for each marker may be associated with the effectiveness of that marker by itself. For example, a ROC curve may be generated for the single marker, and the area under the ROC curve may be used as the initial weighting coefficient for that marker.

Next, a panel response may be calculated for each subject in each of the two sets. The panel response is a function of the indicators to which each marker level is mapped and the weighting coefficients for each marker. In a preferred embodiment, the panel response (R) for a each subject 0) is expressed as:

Rⱼ=ΣwᵢI_{i,j,}

where i is the marker index, j is the subject index, wᵢ is the weighting coefficient for marker i, I is the indicator value to which the marker level for marker i is mapped for subject j, and Σ is the summation over all candidate markers i.

One advantage of using an indicator value rather than the marker value is that an extraordinarily high or low marker levels do not change the probability of a diagnosis of diseased or non-diseased for that particular marker. Typically, a marker value above a certain level generally indicates a certain condition state. Marker values above that level indicate the condition state with the same certainty. Thus, an extraordinarily high marker value may not indicate an extraordinarily high probability of that condition state. The use of an indicator which is constant on one side of the cutoff region eliminates this concern.

The panel response may also be a general function of several parameters including the marker levels and other factors including, for example, race and gender of the patient. Other factors contributing to the panel response may include the slope of the value of a particular marker over time. For example, a patient may be measured when first arriving at the hospital for a particular marker. The same marker may be measured again an hour later, and the level of change may be reflected in the panel response. Further, additional markers may be derived from other markers and may contribute to the value of the panel response. For example, the ratio of values of two markers may be a factor in calculating the panel response.

Having obtained panel responses for each subject in each set of subjects, the distribution of the panel responses for each set may now be analyzed. An objective function may be defined to facilitate the selection of an effective panel. The objective function should generally be indicative of the effectiveness of the panel, as may be expressed by, for example, overlap of the panel responses of the diseased set of subjects and the panel responses of the non-diseased set of subjects. In this manner, the objective function may be optimized to maximize the effectiveness of the panel by, for example, minimizing the overlap.

In a preferred embodiment, the ROC curve representing the panel responses of the two sets of subjects may be used to define the objective function. For example, the objective function may reflect the area under the ROC curve. By maximizing the area under the curve, one may maximize the effectiveness of the panel of markers. In other embodiments, other features of the ROC curve may be used to define the objective function. For example, the point at which the slope of the ROC curve is equal to one may be a useful feature. In other embodiments, the point at which the product of sensitivity and specificity is a maximum, sometimes referred to as the “knee,” may be used. In an embodiment, the sensitivity at the knee may be maximized. In further embodiments, the sensitivity at a predetermined specificity level may be used to define the objective function. Other embodiments may use the specificity at a predetermined sensitivity level may be used. In still other embodiments, combinations of two or more of these ROC-curve features may be used.

It is possible that one of the markers in the panel is specific to the disease or condition being diagnosed. When such markers are present at above or below a certain threshold, the panel response may be set to return a “positive” test result. When the threshold is not satisfied, however, the levels of the marker may nevertheless be used as possible contributors to the objective function.

An optimization algorithm may be used to maximize or minimize the objective function. Optimization algorithms are well-known to those skilled in the art and include several commonly available minimizing or maximizing functions including the Simplex method and other constrained optimization techniques. It is understood by those skilled in the art that some minimization functions are better than others at searching for global minimums, rather than local minimums. In the optimization process, the location and size of the cutoff region for each marker may be allowed to vary to provide at least two degrees of freedom per marker. Such variable parameters are referred to herein as independent variables. In a preferred embodiment, the weighting coefficient for each marker is also allowed to vary across iterations of the optimization algorithm. In various embodiments, any permutation of these parameters may be used as independent variables.

In addition to the above-described parameters, the sense of each marker may also be used as an independent variable. For example, in many cases, it may not be known whether a higher level for a certain marker is generally indicative of a diseased state or a non-diseased state. In such a case, it may be useful to allow the optimization process to search on both sides. In practice, this may be implemented in several ways. For example, in one embodiment, the sense may be a truly separate independent variable which may be flipped between positive and negative by the optimization process. Alternatively, the sense may be implemented by allowing the weighting coefficient to be negative.

The optimization algorithm may be provided with certain constraints as well. For example, the resulting ROC curve may be constrained to provide an area-under-curve of greater than a particular value. ROC curves having an area under the curve of 0.5 indicate complete randomness, while an area under the curve of 1.0 reflects perfect separation of the two sets. Thus, a minimum acceptable value, such as 0.75, may be used as a constraint, particularly if the objective function does not incorporate the area under the curve. Other constraints may include limitations on the weighting coefficients of particular markers. Additional constraints may limit the sum of all the weighting coefficients to a particular value, such as 1.0.

The iterations of the optimization algorithm generally vary the independent parameters to satisfy the constraints while minimizing or maximizing the objective function. The number of iterations may be limited in the optimization process. Further, the optimization process may be terminated when the difference in the objective function between two consecutive iterations is below a predetermined threshold, thereby indicating that the optimization algorithm has reached a region of a local minimum or a maximum.

Thus, the optimization process may provide a panel of markers including weighting coefficients for each marker and cutoff regions for the mapping of marker values to indicators. In order to develop lower-cost panels which require the measurement of fewer marker levels, certain markers may be eliminated from the panel. In this regard, the effective contribution of each marker in the panel may be determined to identify the relative importance of the markers. In one embodiment, the weighting coefficients resulting from the optimization process may be used to determine the relative importance of each marker. The markers with the lowest coefficients may be eliminated.

In certain cases, the lower weighting coefficients may not be indicative of a low importance. Similarly, a higher weighting coefficient may not be indicative of a high importance. For example, the optimization process may result in a high coefficient if the associated marker is irrelevant to the diagnosis. In this instance, there may not be any advantage that will drive the coefficient lower. Varying this coefficient may not affect the value of the objective function.

Exemplary Marker Panels

The present invention is described hereinafter generally in terms of the differential diagnosis of diseases related to dyspnea. The skilled artisan will understand, however, that the concepts of symptom-based differential diagnosis described herein are generally applicable to any physical characteristics that are indicative of a plurality of possible etiologies such as fever, neurologic dysfunction, chest pain (“angina”), dizzyness, headache, etc.

A first step in the identification of suitable markers for symptom-bases differential diagnosis requires a consideration of the possible diagnoses that may be causative of the non-specific symptom observed. In the case of dyspnea, the potential causes are myriad. In a preferred embodiment, the following discussion considers three potential diagnoses: congestive heart failure, pulmonary embolism, and myocardial infarction; and three potential markers for inclusion in a differential diagnosis panel for these potential diagnoses: BNP, D-dimer, and cardiac troponin, respecitively. In another preferred embodiement, markers for three potential diagnoses, congestive heart failure, pulmonary embolism, and myocardial infarction include three potential markers in a differential diagnosis panel, BNP related peptides, D-dimer, and cardiac troponin, respecitively. In a preferred embodiment, three potential diagnoses in the case of dyspnea include congestive heart failure, pulmonary embolism, and myocaridal infarction. In a second preferred embodiment, four potential diagnoses in the case of dyspnea include congestive heart failure, pulmonary embolism, and myocaridal infarction, and atrial fibrillation. Potential markers for inclusion in a differential diagnosis panel include one or more of the following: BNP (heart failure), BNP related peptides (heart failure), D-dimer (pulmonary embolism), cardiac troponin (myocardial infarction), ANP (atrial fibrillation), and ANP related peptides (atrial fibrillation).

BNP

B-type natriuretic peptide (BNP), also called brain-type natriuretic peptide is a 32 amino acid, 4 kDa peptide that is involved in the natriuresis system to regulate blood pressure and fluid balance. Bonow, R. O., Circulation 93:1946-1950 (1996). The precursor to BNP is synthesized as a 108-amino acid molecule, referred to as “pre pro BNP,” that is proteolytically processed into a 76-amino acid N-terminal peptide (amino acids 1-76), referred to as “NT pro BNP” and the 32-amino acid mature hormone, referred to as BNP or BNP 32 (amino acids 77-108). It has been suggested that each of these species—NT pro-BNP, BNP-32, and the pre pro BNP—can circulate in human plasma. Tateyama et al., Biochem. Biophys. Res. Commun. 185: 760-7 (1992); Hunt et al., Biochem. Biophys. Res. Commun. 214: 1175-83 (1995). The 2 forms, pre pro BNP and NT pro BNP, and peptides which are derived from BNP, pre pro BNP and NT pro BNP and which are present in the blood as a result of proteolyses of BNP, NT pro BNP and pre pro BNP, are collectively described as markers related to or associated with BNP.

The term “BNP” as used herein refers to the mature 32-amino acid BNP molecule itself. As the skilled artisan will recognize, however, because of its relationship to BNP, the concentration of NT pro-BNP molecule can also provide diagnostic or prognostic information in patients. The phrases “marker related to BNP” or “BNP related peptide” refers to any polypeptide that originates from the pre pro-BNP molecule, other than the 32-amino acid BNP molecule itself. Proteolytic degradation of BNP and of peptides related to BNP have also been described in the literature and these proteolytic fragments are also encompassed it the term “BNP related peptides.”

BNP and BNP-related peptides are predominantly found in the secretory granules of the cardiac ventricles, and are released from the heart in response to both ventricular volume expansion and pressure overload. Wilkins, M. et al., Lancet 349: 1307-10 (1997). Elevations of BNP are associated with raised atrial and pulmonary wedge pressures, reduced ventricular systolic and diastolic function, left ventricular hypertrophy, and myocardial infarction. Sagnella, G. A., Clinical Science 95: 519-29 (1998). Furthermore, there are numerous reports of elevated BNP concentration associated with congestive heart failure and renal failure. Thus, BNP levels in a patient may be indicative of several possible underlying causes of dyspnea.

D-Dimer

D-dimer is a crosslinked fibrin degradation product with an approximate molecular mass of 200 kDa. The normal plasma concentration of D-dimer is <150 ng/ml (750 pM). The plasma concentration of D-dimer is elevated in patients with acute myocardial infarction and unstable angina, but not stable angina. Hoffmeister, H. M. et al., Circulation 91: 2520-27 (1995); Bayes-Genis, A. et al., Thromb. Haemost. 81: 865-68 (1999); Gurfinkel, E. et al., Br. Heart J. 71: 151-55 (1994); Kruskal, J. B. et al., N. Engl. J. Med. 317: 1361-65 (1987); Tanaka, M. and Suzuki, A., Thromb. Res. 76: 289-98 (1994).

The plasma concentration of D-dimer also will be elevated during any condition associated with coagulation and fibrinolysis activation, including stroke, surgery, atherosclerosis, trauma, and thrombotic thrombocytopenic purpura. D-dimer is released into the bloodstream immediately following proteolytic clot dissolution by plasmin. The plasma concentration of D-dimer can exceed 2 μg/ml in patients with unstable angina. Gurfinkel, E. et al., Br. Heart J 71: 151-55 (1994). Plasma D-dimer is a specific marker of fibrinolysis and indicates the presence of a prothrombotic state associated with acute myocardial infarction and unstable angina. The plasma concentration of D-dimer is also nearly always elevated in patients with acute pulmonary embolism; thus, normal levels of D-dimer may allow the exclusion of pulmonary embolism. Egermayer et al., Thorax 53: 830-34 (1998).

Cardiac Troponin

Troponin I (TnI) is a 25 kDa inhibitory element of the troponin complex, found in muscle tissue. TnI binds to actin in the absence of Ca²⁺, inhibiting the ATPase activity of actomyosin. A TnI isoform that is found in cardiac tissue (cTnI) is 40% divergent from skeletal muscle TnI, allowing both isoforms to be immunologically distinguished. The normal plasma concentration of cTnI is <0.1 ng/ml (4 pM). cTnI is released into the bloodstream following cardiac cell death; thus, the plasma cTnI concentration is elevated in patients with acute myocardial infarction. Investigations into changes in the plasma cTnI concentration in patients with unstable angina have yielded mixed results, but cTnI is not elevated in the plasma of individuals with stable angina. Benamer, H. et al., Am. J. Cardiol. 82: 845-50 (1998); Bertinchant, J. P. et al., Clin. Biochem. 29: 587-94 (1996); Tanasijevic, M. J. et al., Clin. Cardiol. 22: 13-16 (1999); Musso, P. et al., J. Ital. Cardiol. 26: 1013-23 (1996); Holvoet, P. et al., JAMA 281: 1718-21 (1999); Holvoet, P. et al., Circulation 98: 1487-94 (1998).

The plasma concentration of cTnI in patients with acute myocardial infarction is significantly elevated 4-6 hours after onset, peaks between 12-16 hours, and can remain elevated for one week. The release kinetics of cTnI associated with unstable angina may be similar. The measurement of specific forms of cardiac troponin, including free cardiac troponin I and complexes of cardiac troponin I with troponin C and/or T may provide the user with the ability to identify various stages of ACS. Free and complexed cardiac-troponin T may be used in a manner analogous to that described for cardiac troponin I. Cardiac troponin T complex may be useful either alone or when expressed as a ratio with total cardiac troponin I to provide information related to the presence of progressing myocardial damage. Ongoing ischemia may result in the release of the cardiac troponin TIC complex, indicating that higher ratios of cardiac troponin TIC:total cardiac troponin I may be indicative of continual damage caused by unresolved ischemia. See, U.S. Pat. Nos. 6,147,688, 6,156,521, 5,947,124, and 5,795,725, which are hereby incorporated by reference in their entirety, including all tables, figures, and claims. One skilled in the art recognizes that in measuring cardiac troponin, one can measure the different isoforms of troponin I and troponin T.

One skilled in the art recognizes that in measuring cardiac troponin, one can measure the different isoforms of troponin I and troponin T. Thus, one may preferably measure free cardiac troponin I, free cardiac troponin T, cardiac troponin I in a complex comprising one or both of troponin T and troponin C, cardiac troponin T in a complex comprising one or both of troponin I and troponin C, total cardiac troponin I (meaning free and complexed cardiac troponin I), and/or total cardiac troponin T, The term “at least one cardiac troponin form” as used herein refers to any one of these foregoing forms.

ANP

A-type natriuretic peptide (ANP) (also referred to as atrial natriuretic peptide or cardiodilatin Forssmann et al Histochem Cell Biol 110: 335-357 (1998)) is a 28 amino acid peptide that is synthesized, stored, and released atrial myocytes in response to atrial distension, angiotensin II stimulation, endothelin, and sympathetic stimulation (beta-adrenoceptor mediated). ANP is synthesized as a precursor molecule (pro-ANP) that is converted to an active form, ANP, by proteolytic cleavage and also forming N-terminal ANP (1-98). N-terminal ANP and ANP have been reported to increase in patients exhibiting atrial fibrillation and heart failure (Rossi et al. Journal of the American College of Cardiology 35: 1256-62 (2000). In addition to atrial natriuretic peptide (ANP99-126) itself, linear peptide fragments from its N-terminal prohormone segment have also been reported to have biological activity. As the skilled artisan will recognize, however, because of its relationship to ANP, the concentration of N-terminal ANP molecule can also provide diagnostic or prognostic information in patients. The phrase “marker related to ANP or ANP related peptide” refers to any polypeptide that originates from the pro-ANP molecule (1-126), other than the 28-amino acid ANP molecule itself. Proteolytic degradation of ANP and of peptides related to ANP have also been described in the literature and these proteolytic fragments are also encompassed it the term “ANP related peptides.”

Elevated levels of ANP are found during hypervolemia, atrial fibrillation and congestive heart failure. ANP is involved in the long-term regulation of sodium and water balance, blood volume and arterial pressure. This hormone decreases aldosterone release by the adrenal cortex, increases glomerular filtration rate (GFR), produces natriuresis and diuresis (potassium sparing), and decreases renin release thereby decreasing angiotensin II. These actions contribute to reductions in blood volume and therefore central venous pressure (CVP), cardiac output, and arterial blood pressure. Several isoforms of ANP have been identified, and their relationship to stroke incidence studied. See, e.g., Rubatu et al., Circulation 100:1722-6, 1999; Estrada et al., Am. J. Hypertens. 7:1085-9, 1994.

Chronic elevations of ANP appear to decrease arterial blood pressure primarily by decreasing systemic vascular resistance. The mechanism of systemic vasodilation may involve ANP receptor-mediated elevations in vascular smooth muscle cGMP as well as by attenuating sympathetic vascular tone. This latter mechanism may involve ANP acting upon sites within the central nervous system as well as through inhibition of norepinephrine release by sympathetic nerve terminals. ANP may be viewed as a counter-regulatory system for the renin-angiotensin system. A new class of drugs that are neutral endopeptidase (NEP) inhibitors have demonstrated efficacy in heart failure. These drugs inhibit neutral endopeptidase, the enzyme responsible for the degradation of ANP, and thereby elevate plasma levels of ANP. NEP inhibition is particularly effective in heart failure when the drug has a combination of both NEP and ACE inhibitor properties.

Based on the foregoing discussion, the skilled artisan will recognize that, for example, increased BNP is indicative of congestive heart failure, but may also be indicative of other cardiac-related conditions such as myocardial infarction. Thus, the inclusion of a marker related to myocardial injury such as cardiac troponin I and/or cardiac troponin T can permit further discrimination of the disease underlying the observed dyspnea and the increased BNP level. In this case, an increased level of cardiac troponin may be used to rule in myocardial infarction.

Similarly, BNP may also be indicative of pulmonary embolism. The inclusion of a marker related to coagulation and hemostasis such as D-dimer can permit further discrimination of the disease underlying the observed dyspnea and the increased BNP level. In this case, a normal level of D-dimer may be used to rule out pulmonary embolism.

A detailed analysis of this exemplary marker panel is provided in the following examples. The skilled artisan will readily acknowledge that other markers may be substituted in or added to this marker panel to further discriminate the causes of dyspnea in accordance with the methods for identification and use of diagnostic markers described herein. Additional suitable markers are described in the following sections.

As discussed in detail herein, the foregoing principles of marker panel design may be applied broadly to symptom-based differential diagnosis. For example, in the case of abdominal pain, the plurality of markers are preferably selected to rule in or out a plurality of the following: aortic dissection, mesenteric embolism, pancreatitis, appendicitis, angina, myocardial infarction, one or more infectious diseases described above, influenza, esophageal carcinoma, gastric adenocarcinoma, colorectal adenocarcinoma, pancreatic tumors including ductal adenocarcinoma, cystadenocarcinoma, and insulinoma. In a preferred embodiment, the potential diagnoses for abdominal pain include aortic aneurysm, mesenteric embolism, pancreatitis, appendicitis, angina and myocardial infarction.

The foregoing principles may also be applied to subdivide differential diagnosis to a given level of detail required by the clinical artisan. For example, the differential diagnosis of various symptoms may require discrimination between heart failure and atrial fibrillation. An exemplary marker panel for performing such discrimination preferably includes BNP or BNP related peptides, and ANP or ANP related peptides, respectively. Additional markers may be defined to distinguish between systolic and diastolic dysfunction and atrial fibrillation. Preferred markers in this case include BNP, calcitonin gene related peptide, calcitonin and urotensin 1 for differentiation of systolic and diastolic dysfunction and ANP or ANP related peptides for the detection of atrial fibrillation. Likewise, markers may be defined to distinguish between systolic and diastolic dysfunction, atrial fibrillation, myocardial ischemia and cardiac necrosis. Preferred markers in this case include BNP, calcitonin gene related peptide, calcitonin and urotensin 1 for differentiation of systolic and diastolic dysfunction and ANP or ANP related peptides for the detection of atrial fibrillation and BNP and cardiac troponins for the detection of myocardial ischemia and necrosis.

In the case of chest pain, the present invention can provide markers able to distinguish between aortic dissection, myocardial ischemia, and cardiac necrosis; markers able to distinguish between aortic dissection, myocardial ischemia, and myocardial infarction; markers able to distinguish between aortic dissection, myocardial ischemia, cardiac necrosis and heart failure; markers able to distinguish between aortic dissection, myocardial ischemia, cardiac necrosis and myocardial infarction; markers able to distinguish between aortic dissection, myocardial ischemia, cardiac necrosis and atrial fibrillation; and/or markers able to distinguish between aortic dissection, myocardial ischemia and cardiac necrosis, myocardial infarction and atrial fibrillation. In accordance with the foregoing, a particularly preferred marker for aortic dissection is smooth muscle myosin, and most preferably smooth muscle myosin heavy chain, and a particularly preferred marker for atrial fibrillation is ANP or an ANP-related marker.

Preferred marker sets are those comprising smooth muscle myosin heavy chain and ANP or an ANP-related marker to distinguish aortic dissection and atrial fibrillation, respectively; smooth muscle myosin heavy chain, ANP or an ANP-related marker, and BNP or a BNP-related marker to distinguish aortic dissection, atrial fibrillation and myocardial ischemia, respectively; smooth muscle myosin heavy chain, BNP or a BNP-related marker, and a cardiac troponin form to distinguish aortic dissection, myocardial ischemia, and myocardial infarction, respectively; and smooth muscle myosin heavy chain, BNP or a BNP-related marker, creatine kinase MB, myoglobin, and a cardiac troponin form to distinguish aortic dissection, myocardial ischemia, cardiac necrosis, and myocardial infarction.

Similarly, in the case of disturbanes of metabolic state, the plurality of markers are preferably selected to rule in or out a plurality of the following: diabetes mellitus, diabetic ketoacidosis, alcoholic ketoacidosis, respiratory acidosis, respiratory alkalosis, nonketogenic hyperglycemia, hypoglycemia, renal failure, interstitial renal disease, COPD, pneumonia, pulmonary edema and asthma.

In the case of neurologic dysfunction, the plurality of markers are preferably selected to rule in or out a plurality of the following: stroke, brain tumor, cerebral hypoxia, hypoglycemia, migraine, atrial fibrillation, myocardial infarction, cardiac ischemia, peripheral vascular disease and seizure. Preferred markers in this case include specific markers of cerebral injury such as adenylate kinase, brain-derived neurotrophic factor, calbindin-D, creatine kinase-BB, glial fibrillary acidic protein, lactate dehydrogenase, myelin basic protein, neural cell adhesion molecule, neuron-specific enolase, neurotrophin-3, proteolipid protein, S-100b, thrombomodulin, protein kinase C gamma; and/or one or more non-specific markers of cerebral injury such as β-thromboglobulin, D-dimer, fibrinopeptide A, plasmin-α-2-antiplasmin complex, platelet factor 4, prothrombin fragment 1+2, thrombin-antithrombin III complex, tissue factor, von Willebrand factor, adrenomedullin, cardiac troponin I (for myocardial ischemia and necrosis), head activator, hemoglobin α₂ chain, caspase-3, vascular endothelial growth factor (VEGF), one or more endothelins (e.g., endothelin-1, endothelin-2, and endothelin-3), interleukin-8, Atrial natriuretic peptide, B-type natriuretic peptide (for myocardial ischemia and necrosis), and C-type natriuretic peptide; and/or one or more acute phase reactants such as C-reactive protein, ceruloplasmin, fibrinogen, α1-acid glycoprotein, α₂-antitrypsin, haptoglobin, insulin-like growth factor-1, interleukin-1β, interleukin-1 receptor antagonist, interleukin-6, transforming growth factor, tumor necrosis factor α, E-selectin, intercellular adhesion molecule-1, matrix metalloproteinases (e.g., matrix metalloproteinase 9 (MMP-9)), monocyte chemotactic protein-1, and vascular cell adhesion molecule.

Methods and marker sets for differential diagnosis of stroke and other cerebral injuries are described in U.S. patent Ser. No. 10/225,082, filed Aug. 20, 2002, which is hereby incorporated in its entirety, including all tables figures and claims. As described therein, preferred marker panels diagnose and/or differentiate between stroke, subarachnoid hemorrhage, intracerebral hemorrhage, and/or hemorrhagic stroke; and/or can distinguish between ischemic and hemorrhagic stroke. Particularly preferred are markers that differentiate between thrombotic, embolic, lacunar, hypoperfusion, intracerebral hemorrhage, and subarachnoid hemorrhage types of strokes. Particularly preferred marker sets include BNP, IL-6, S-100b, MMP-9, TAT complex, and vWF A1-integrin; BNP, S-100b, MMP-9, and vWF-A1-integrin; vWF-A1, VEGF, and MMP-9; caspase-3, MMP-9, and GFAP; caspase-3, MMP-9, vWF-A1, and BNP; NCAM, BDNF, Caspase-3, MMP-9, vWF-A1, and VEGF; NCAM, BDNF, Caspase-3, MMP-9, vWF-A1, and S-100b; VEGF; NCAM, BDNF, Caspase-3, MMP-9, vWF-A1, and MCP-1; VEGF; NCAM, BDNF, Caspase-3, MMP-9, VEGF, and vWF A1-integrin; BDNF, MMP-9, S-100b, vWF A1-integrin, MCP-1, and GFAP; BDNF, caspase-3, MMP-9, vWF-A1, S-100b, and GFAP; NCAM, BDNF, MMP-9, vWF-A1, S-100b, and GFAP; NCAM, BDNF, caspase-3, MMP-9, S-100P, and GFAP; caspase-3, NCAM, MCP-1, S100b, MMP-9, vWF A1-integrin, and BNP; caspase-3, NCAM, MCP-1, S100b, MMP-9, vWF A1, BNP, and GFAP; CRP, NT-3, vWF, MMP-9, VEGF, and CKBB; CRP, MMP-9, VEGF, CKBB, and MCP-1; CRP, NT-3, MMP-9, VEGF, CKBB, and MCP-1; and CRP, MMP-9, VEGF, CKBB, MCP-1. Calbindin, vWF VP1, vWF A3, vWF A1-A3, TAT complex, proteolipid protein, IL-6, IL-8, myelin basic protein, S-100b, tissue factor, GFAP, vWF A1-integrin, CNP, and NCAM.

A panel consisting of the markers referenced herein may be constructed to provide relevant information related to the differential diagnosis of interest. Such a panel may be constructed using 1, 2, 3, 4, 5, 6, 7, 8, 9, 10, 11, 12, 13, 14, 15, 16, 17, 18, 19 or 20 individual markers. The analysis of a single marker or subsets of markers comprising a larger panel of markers could be carried out by one skilled in the art to optimize clinical sensitivity or specificity in various clinical settings. These include, but are not limited to ambulatory, urgent care, critical care, intensive care, monitoring unit, inpatient, outpatient, physician office, medical clinic, and health screening settings. Furthermore, one skilled in the art can use a single marker or a subset of markers comprising a larger panel of markers in combination with an adjustment of the diagnostic threshold in each of the aforementioned settings to optimize clinical sensitivity and specificity. The clinical sensitivity of an assay is defined as the percentage of those with the disease that the assay correctly predicts, and the specificity of an assay is defined as the percentage of those without the disease that the assay corrects predicts (Tietz Textbook of Clinical Chemistry, 2^{nd} edition, Carl Burtis and Edward Ashwood eds., W.B. Saunders and Company, p. 496). The following provides a brief discussion of additional exemplary markers for use in identifying suitable marker panels by the methods described herein.

(i) Exemplary Markers Related to Myocardial Injury

Annexin V, also called lipocortin V, endonexin II, calphobindin I, calcium binding protein 33, placental anticoagulant protein I, thromboplastin inhibitor, vascular anticoagulant-α, and anchorin CII, is a 33 kDa calcium-binding protein that is an indirect inhibitor and regulator of tissue factor. Annexin V is composed of four homologous repeats with a consensus sequence common to all annexin family members, binds calcium and phosphatidyl serine, and is expressed in a wide variety of tissues, including heart, skeletal muscle, liver, and endothelial cells (Giambanco, I. et al., J. Histochem. Cytochem. 39:P1189-1198, 1991; Doubell, A. F. et al., Cardiovasc. Res. 27:1359-1367, 1993). The normal plasma concentration of annexin V is <2 ng/ml (Kaneko, N. et al., Clin. Chim. Acta 251:65-80, 1996). The plasma concentration of annexin V is elevated in individuals with acute myocardial infarction (Kaneko, N. et al., Clin. Chim. Acta 251:65-80, 1996). Due to its wide tissue distribution, elevation of the plasma concentration of annexin V may be associated with any condition involving non-cardiac tissue injury. However, one study has found that plasma annexin V concentrations were not significantly elevated in patients with old myocardial infarction, chest pain syndrome, valvular heart disease, lung disease, and kidney disease (Kaneko, N. et al., Clin. Chim. Acta 251:65-80, 1996). Annexin V is released into the bloodstream soon after acute myocardial infarction onset. The annexin V concentration in the plasma of acute myocardial infarction patients decreased from initial (admission) values, suggesting that it is rapidly cleared from the bloodstream (Kaneko, N. et al. Clin. Chim. Acta 251:65-80, 1996).

Enolase is a 78 kDa homo- or heterodimeric cytosolic protein produced from α, β, and γ subunits. Enolase catalyzes the interconversion of 2-phosphoglycerate and phosphoenolpyruvate in the glycolytic pathway. Enolase is present as αα, αβ, ββ, αγ and γγ isoforms. The α subunit is found in most tissues, the β subunit is found in cardiac and skeletal muscle, and the γ subunit is found primarily in neuronal and neuroendocrine tissues. β-enolase is composed of αβ and ββ enolase, and is specific for muscle. The normal plasma concentration of β-enolase is <10 ng/ml (120 pM). β-enolase is elevated in the serum of individuals with acute myocardial infarction, but not in individuals with angina (Nomura, M. et al., Br. Heart J. 58:29-33, 1987; Herraez-Dominguez, M. V. et al., Clin. Chim. Acta 64:307-315, 1975). Further investigations into possible changes in plasma β-enolase concentration associated with unstable and stable angina need to be performed. The plasma concentration of β-enolase is elevated during heart surgery, muscular dystrophy, and skeletal muscle injury (Usui, A. et al., Cardiovasc. Res. 23:737-740, 1989; Kato, K. et al., Clin. Chim. Acta 131:75-85, 1983; Matsuda, H. et al., Forensic Sci. Int. 99:197-208, 1999). β-enolase is released into the bloodstream immediately following cardiac or skeletal muscle injury. The plasma β-enolase concentration was elevated to more than 150 ng/ml in the perioperative stage of cardiac surgery, and remained elevated for 1 week. Serum β-enolase concentrations peaked approximately 12-14 hours after the onset of chest pain and acute myocardial infarction and approached baseline after 1 week had elapsed from onset, with maximum levels approaching 1 μg/ml (Kato, K. et al., Clin. Chim. Acta 131:75-85, 1983; Nomura, M. et al., Br. Heart J. 58:29-33, 1987).

Creatine kinase (CK) is a 85 kDa cytosolic enzyme that catalyzes the reversible formation ADP and phosphocreatine from ATP and creatine. CK is a homo- or heterodimer composed of M and B chains. CK-MB is the isoform that is most specific for cardiac tissue, but it is also present in skeletal muscle and other tissues. The normal plasma concentration of CK-MB is <5 ng/ml. The plasma CK-MB concentration is significantly elevated in patients with acute myocardial infarction. Plasma CK-MB is not elevated in patients with stable angina, and investigation into plasma CK-MB concentration elevations in patients with unstable angina have yielded mixed results (Thygesen, K. et al., Eur. J. Clin. Invest. 16:1-4, 1986; Koukkunen, H. et al., Ann. Med. 30:488-496, 1998; Bertinchant, J. P. et al., Clin. Biochem. 29:587-594, 1996; Benamer, H. et al., Am. J. Cardiol. 82:845-850, 1998; Norregaard-Hansen, K. et al., Eur. Heart J. 13:188-193, 1992). The mixed results associated with unstable angina suggest that CK-MB may be useful in determining the severity of unstable angina because the extent of myocardial ischemia is directly proportional to unstable angina severity. Elevations of the plasma CK-MB concentration are associated with skeletal muscle injury and renal disease. CK-MB is released into the bloodstream following cardiac cell death. The plasma concentration of CK-MB in patients with acute myocardial infarction is significantly elevated 4-6 hours after onset, peaks between 12-24 hours, and returns to baseline after 3 days. The release kinetics of CK-MB associated with unstable angina may be similar.

Glycogen phosphorylase (GP) is a 188 kDa intracellular allosteric enzyme that catalyzes the removal of glucose (liberated as glucose-1-phosphate) from the nonreducing ends of glycogen in the presence of inorganic phosphate during glycogenolysis. GP is present as a homodimer, which associates with another homodimer to form a tetrameric enzymatically active phosphorylase A. There are three isoforms of GP that can be immunologically distinguished. The BB isoform is found in brain and cardiac tissue, the MM isoform is found in skeletal muscle and cardiac tissue, and the LL isoform is predominantly found in liver (Mair, J. et al., Br. Heart J. 72:125-127, 1994). GP-BB is normally associated with the sarcoplasmic reticulum glycogenolysis complex, and this association is dependent upon the metabolic state of the myocardium (Mair, J., Clin. Chim. Acta 272:79-86, 1998). At the onset of hypoxia, glycogen is broken down, and GP-BB is converted from a bound form to a free cytoplasmic form (Krause, E. G. et al. Mol. Cell Biochem. 160-161:289-295, 1996). The normal plasma GP-BB concentration is <7 ng/ml (36 pM). The plasma GP-BB concentration is significantly elevated in patients with acute myocardial infarction and unstable angina with transient ST-T elevations, but not stable angina (Mair, J. et al., Br. Heart J. 72:125-127, 1994; Mair, J., Clin. Chim. Acta 272:79-86, 1998; Rabitzsch, G. et al., Clin. Chem. 41:966-978, 1995; Rabitzsch, G. et al., Lancet 341:1032-1033, 1993). Furthermore, GP-BB also can be used to detect perioperative acute myocardial infarction and myocardial ischemia in patients undergoing coronary artery bypass surgery (Rabitzsch, G. et al., Biomed. Biochim. Acta 46:S584-S588, 1987; Mair, P. et al., Eur. J. Clin. Chem. Clin. Biochem. 32:543-547, 1994). GP-BB has been demonstrated to be a more sensitive marker of unstable angina and acute myocardial infarction early after onset than CK-MB, cardiac tropopnin T, and myoglobin (Rabitzsch, G. et al., Clin. Chem. 41:966-978, 1995). Because it is also found in the brain, the plasma GP-BB concentration also may be elevated during ischemic cerebral injury. GP-BB is released into the bloodstream under ischemic conditions that also involve an increase in the permeability of the cell membrane, usually a result of cellular necrosis. GP-BB is significantly elevated within 4 hours of chest pain onset in individuals with unstable angina and transient ST-T ECG alterations, and is significantly elevated while myoglobin, CK-MB, and cardiac troponin T are still within normal levels (Mair, J. et al., Br. Heart J 72:125-127, 1994). Furthermore, GP-BB can be significantly elevated 1-2 hours after chest pain onset in patients with acute myocardial infarction (Rabitzsch, G. et al., Lancet 341:1032-1033, 1993). The plasma GP-BB concentration in patients with unstable angina and acute myocardial infarction can exceed 50 ng/ml (250 pM) (Mair, J. et al., Br. Heart J 72:125-127, 1994; Mair, J., Clin. Chim. Acta 272:79-86, 1998; Krause, E. G. et al., Mol. Cell Biochem. 160-161:289-295, 1996; Rabitzsch, G. et al., Clin. Chem. 41:966-978, 1995; Rabitzsch, G. et al., Lancet 341:1032-1033, 1993). GP-BB appears to be a very sensitive marker of myocardial ischemia, with specificity similar to that of CK-BB. GP-BB plasma concentrations are elevated within the first 4 hours after acute myocardial infarction onset, which suggests that it may be a very useful early marker of myocardial damage. Furthermore, GP-BB is not only a more specific marker of cardiac tissue damage, but also ischemia, since it is released to an unbound form during myocardial ischemia and would not normally be released upon traumatic injury. This is best illustrated by the usefulness of GP-BB in detecting myocardial ischemia during cardiac surgery. GP-BB may be a very useful marker of early myocardial ischemia during acute myocardial infarction and severe unstable angina.

Heart-type fatty acid binding protein (H-FABP) is a cytosolic 15 kDa lipid-binding protein involved in lipid metabolism. Heart-type FABP antigen is found not only in heart tissue, but also in kidney, skeletal muscle, aorta, adrenals, placenta, and brain (Veerkamp, J. H. and Maatman, R. G., Prog. Lipid Res. 34:17-52, 1995; Yoshimoto, K. et al., Heart Vessels 10:304-309, 1995). Furthermore, heart-type FABP mRNA can be found in testes, ovary, lung, mammary gland, and stomach (Veerkamp, J. H. and Maatman, R. G., Prog. Lipid Res. 34:17-52, 1995). The normal plasma concentration of FABP is <6 ng/ml (400 pM). The plasma H-FABP concentration is elevated in patients with acute myocardial infarction and unstable angina (Ishii, J. et al., Clin. Chem. 43:1372-1378, 1997; Tsuji, R. et al., Int. J. Cardiol. 41:209-217, 1993). Furthermore, H-FABP may be useful in estimating infarct size in patients with acute myocardial infarction (Glatz, J. F. et al., Br. Heart J. 71:135-140, 1994). Myocardial tissue as a source of H-FABP can be confirmed by determining the ratio of myoglobin/FABP (grams/grams). A ratio of approximately 5 indicates that FABP is of myocardial origin, while a higher ratio indicates skeletal muscle sources (Van Nieuwenhoven, F. A. et al., Circulation 92:2848-2854, 1995). Because of the presence of H-FABP in skeletal muscle, kidney and brain, elevations in the plasma H-FABP concentration may be associated with skeletal muscle injury, renal disease, or stroke. H-FABP is released into the bloodstream following cardiac tissue necrosis. The plasma H-FABP concentration can be significantly elevated 1-2 hours after the onset of chest pain, earlier than CK-MB and myoglobin (Tsuji, R. et al., Int. J. Cardiol. 41:209-217, 1993; Van Nieuwenhoven, F. A. et al., Circulation 92:2848-2854, 1995; Tanaka, T. et al., Clin. Biochem. 24:195-201, 1991). Additionally, H-FABP is rapidly cleared from the bloodstream, and plasma concentrations return to baseline after 24 hours after acute myocardial infarction onset (Glatz, J. F. et al., Br. Heart J. 71:135-140, 1994; Tanaka, T. et al., Clin. Biochem. 24:195-201, 1991).

Phosphoglyceric acid mutase (PGAM) is a 57 kDa homo- or heterodimeric intracellular glycolytic enzyme composed of 29 kDa M or B subunits that catalyzes the interconversion of 3-phosphoglycerate to 2-phosphoglycerate in the presence of magnesium. Cardiac tissue contains isozymes MM, MB, and BB, skeletal muscle contains primarily PGAM-MM, and most other tissues contain PGAM-BB (Durany, N. and Carreras, J., Comp. Biochem. Physiol. B. Biochem. Mol. Biol. 114:217-223, 1996). Thus, PGAM-MB is the most specific isozyme for cardiac tissue. PGAM is elevated in the plasma of patients with acute myocardial infarction, but further studies need to be performed to determine changes in the plasma PGAM concentration associated with acute myocardial infarction, unstable angina and stable angina (Mair, J., Crit. Rev. Clin. Lab. Sci. 34:1-66, 1997). Plasma PGAM-MB concentration elevations may be associated with unrelated myocardial or possibly skeletal tissue damage. PGAM-MB is most likely released into the circulation following cellular necrosis. PGAM has a half-life of less than 2 hours in the bloodstream of rats (Grisolia, S. et al., Physiol. Chem. Phys. 8:37-52, 1976).

S-100 is a 21 kDa homo- or heterodimeric cytosolic Ca²⁺-binding protein produced from α and β subunits. It is thought to participate in the activation of cellular processes along the Ca²⁺-dependent signal transduction pathway (Bonfrer, J. M. et al., Br. J. Cancer 77:2210-2214, 1998). S-100ao (αα isoform) is found in striated muscles, heart and kidney, S-100a (αβ isoform) is found in glial cells, but not in Schwann cells, and S-100b (ββ isoform) is found in high concentrations in glial cells and Schwann cells, where it is a major cytosolic component (Kato, K. and Kimura, S., Biochim. Biophys. Acta 842:146-150, 1985; Hasegawa, S. et al., Eur. Urol. 24:393-396, 1993). The normal serum concentration of S-100ao is <0.25 ng/ml (12 pM), and its concentration may be influenced by age and sex, with higher concentrations in males and older individuals (Kikuchi, T. et al., Hinyokika Kiyo 36:1117-1123, 1990; Morita, T. et al., Nippon Hinyokika Gakkai Zasshi 81:1162-1167, 1990; Usui, A. et al., Clin. Chem. 36:639-641, 1990). The serum concentration of S-100ao is elevated in patients with acute myocardial infarction, but not in patients with angina pectoris with suspected acute myocardial infarction (Usui, A. et al., Clin. Chem. 36:639-641, 1990). Further investigation is needed to determine changes in the plasma concentration of S-100ao associated with unstable and stable angina. Serum S-100ao is elevated in the serum of patients with renal cell carcinoma, bladder tumor, renal failure, and prostate cancer, as well as in patients undergoing open heart surgery (Hasegawa, S. et al., Eur. Urol. 24:393-396, 1993; Kikuchi, T. et al., Hinyokika Kiyo 36:1117-1123, 1990; Morita, T. et al., Nippon Hinyokika Gakkai Zasshi 81:1162-1167, 1990; Usui, A. et al., Clin. Chem. 35:1942-1944, 1989). S-100ao is a cytosolic protein that will be released into the extracellular space following cell death. The serum concentration of S-100ao is significantly elevated on admission in patients with acute myocardial infarction, increases to peak levels 8 hours after admission, decreases and returns to baseline one week later (Usui, A. et al., Clin. Chem. 36:639-641, 1990). Furthermore, S-100ao appears to be significantly elevated earlier after acute myocardial infarction onset than CK-MB (Usui, A. et al., Clin. Chem. 36:639-641, 1990). The maximum serum S-100ao concentration can exceed 100 ng/ml. S-100ao may be rapidly cleared from the bloodstream by the kidney, as suggested by the rapid decrease of the serum S-100ao concentration of heart surgery patients following reperfusion and its increased urine concentration. S-100ao is found in high concentration in cardiac tissue and appears to be a sensitive marker of cardiac injury. Major sources of non-specificity of this marker include skeletal muscle and renal tissue injury. S-100ao may be significantly elevated soon after acute myocardial infarction onset, and it may allow for the discrimination of acute myocardial infarction from unstable angina. Patients with angina pectoris and suspected acute myocardial infarction, indicating that they were suffering chest pain associated with an ischemic episode, did not have a significantly elevated S-100ao concentration.

Myoglobin is a small (˜17.8 kDa) heme-containing protein responsible for the oxygen deposition in muscle tissue. The small molecular weight of myoglobin allows it to be rapidly released from muscle tissue when the tissue is damaged. By comparison, creatine kinase and its isoform CK-MB are larger than myoglobin and are released more slowly after an acute myocardial infarction. Because myoglobin escapes rapidly from damaged myocardial cells, it can be detected as soon as 1 hour after an acute myocardial infarction, with peak serum levels occurring in 3 to 15 hours The same form of myoglobin is expressed in cardiac and in skeletal muscle tissues. Because of its lower cardiac specificity, it is often used together with cardiac Troponin I or Troponin T. Although using early detection of elevated myoglobin levels in patients with chest pain to determine whether an AMI has occurred sounds ideal, some disadvantages are apparent. Because both cardiac and skeletal muscle contain myoglobin, many non-cardiac-related factors, such as skeletal muscle or neuromuscular disorders, strenuous exercise, renal failure, intramuscular injections, and cardiac bypass surgery, can elevate serum levels of the protein. Controversy also exists about the level at which myoglobin becomes indicative of an acute myocardial infarction, with the reported reference ranges varying from 50 to 120 μg/mL. See, e.g., Montague and Kircher, Am J Clin Pathol. 104:472-476 (1995); Bhayana and Henderson, Clin Biochem. 28:1-29 (1995); Wong, Ann Clin Lab Sci. 26:301-312 (1996); and Selker et al., Ann Emerg Med. 29:63-69 (1997).

(ii) Exemplary Markers Related to Coagulation and Hemostasis

Plasmin is a 78 kDa serine proteinase that proteolytically digests crosslinked fibrin, resulting in clot dissolution. The 70 kDa serine proteinase inhibitor α2-antiplasmin α2AP) regulates plasmin activity by forming a covalent 1:1 stoichiometric complex with plasmin. The resulting ˜150 kDa plasmin-α2AP complex (PAP), also called plasmin inhibitory complex (PIC) is formed immediately after α2AP comes in contact with plasmin that is activated during fibrinolysis. The normal serum concentration of PAP is <1 μg/ml (6.9 nM). Elevations in the serum concentration of PAP can be attributed to the activation of fibrinolysis. Elevations in the serum concentration of PAP may be associated with clot presence, or any condition that causes or is a result of fibrinolysis activation. These conditions can include atherosclerosis, disseminated intravascular coagulation, acute myocardial infarction, surgery, trauma, unstable angina, stroke, and thrombotic thrombocytopenic purpura. PAP is formed immediately following proteolytic activation of plasmin. PAP is a specific marker for fibrinolysis activation and the presence of a recent or continual hypercoagulable state.

β-thromboglobulin (βTG) is a 36 kDa platelet α granule component that is released upon platelet activation. The normal plasma concentration of βTG is <40 ng/ml (1.1 nM). Plasma levels of β-TG appear to be elevated in patients with unstable angina and acute myocardial infarction, but not stable angina (De Caterina, R. et al., Eur. Heart J. 9:913-922, 1988; Bazzan, M. et al., Cardiologia 34, 217-220, 1989). Plasma β-TG elevations also seem to be correlated with episodes of ischemia in patients with unstable angina (Sobel, M. et al., Circulation 63:300-306, 1981). Elevations in the plasma concentration of βTG may be associated with clot presence, or any condition that causes platelet activation. These conditions can include atherosclerosis, disseminated intravascular coagulation, surgery, trauma, and thrombotic thrombocytopenic purpura, and stroke (Landi, G. et al., Neurology 37:1667-1671, 1987). βTG is released into the circulation immediately after platelet activation and aggregation. It has a biphasic half-life of 10 minutes, followed by an extended 1 hour half-life in plasma (Switalska, H. I. et al., J. Lab. Clin. Med. 106:690-700, 1985). Plasma βTG concentration is reportedly elevated dring unstable angina and acute myocardial infarction. Special precautions must be taken to avoid platelet activation during the blood sampling process. Platelet activation is common during regular blood sampling, and could lead to artificial elevations of plasma βTG concentration. In addition, the amount of βTG released into the bloodstream is dependent on the platelet count of the individual, which can be quite variable. Plasma concentrations of βTG associated with ACS can approach 70 ng/ml (2 nM), but this value may be influenced by platelet activation during the sampling procedure.

Platelet factor 4 (PF4) is a 40 kDa platelet α granule component that is released upon platelet activation. PF4 is a marker of platelet activation and has the ability to bind and neutralize heparin. The normal plasma concentration of PF4 is <7 ng/ml (175 pM). The plasma concentration of PF4 appears to be elevated in patients with acute myocardial infarction and unstable angina, but not stable angina (Gallino, A. et al., Am. Heart J. 112:285-290, 1986; Sakata, K. et al., Jpn. Circ. J. 60:277-284, 1996; Bazzan, M. et al., Cardiologia 34:217-220, 1989). Plasma PF4 elevations also seem to be correlated with episodes of ischemia in patients with unstable angina (Sobel, M. et al., Circulation 63:300-306, 1981). Elevations in the plasma concentration of PF4 may be associated with clot presence, or any condition that causes platelet activation. These conditions can include atherosclerosis, disseminated intravascular coagulation, surgery, trauma, thrombotic thrombocytopenic purpura, and acute stroke (Carter, A. M. et al., Arterioscler. Thromb. Vasc. Biol. 18:1124-1131, 1998). PF4 is released into the circulation immediately after platelet activation and aggregation. It has a biphasic half-life of 1 minute, followed by an extended 20 minute half-life in plasma. The half-life of PF4 in plasma can be extended to 20-40 minutes by the presence of heparin (Rucinski, B. et al., Am. J. Physiol. 251:H800-H807, 1986). Plasma PF4 concentration is reportedly elevated during unstable angina and acute myocardial infarction, but these studies may not be completely reliable. Special precautions must be taken to avoid platelet activation during the blood sampling process. Platelet activation is common during regular blood sampling, and could lead to artificial elevations of plasma PF4 concentration. In addition, the amount of PF4 released into the bloodstream is dependent on the platelet count of the individual, which can be quite variable. Plasma concentrations of PF4 associated with disease can exceed 100 ng/ml (2.5 nM), but it is likely that this value may be influenced by platelet activation during the sampling procedure.

Fibrinopeptide A (FPA) is a 16 amino acid, 1.5 kDa peptide that is liberated from amino terminus of fibrinogen by the action of thrombin. Fibrinogen is synthesized and secreted by the liver. The normal plasma concentration of FPA is <5 ng/ml (3.3 nM). The plasma FPA concentration is elevated in patients with acute myocardial infarction, unstable angina, and variant angina, but not stable angina (Gensini, G. F. et al., Thromb. Res. 50:517-525, 1988; Gallino, A. et al., Am. Heart J 112:285-290, 1986; Sakata, K. et al., Jpn. Circ. J. 60:277-284, 1996; Theroux, P. et al., Circulation 75:156-162, 1987; Merlini, P. A. et al., Circulation 90:61-68, 1994; Manten, A. et al., Cardiovasc. Res. 40:389-395, 1998). Furthermore, plasma FPA may indicate the severity of angina (Gensini, G. F. et al., Thromb. Res. 50:517-525, 1988). Elevations in the plasma concentration of FPA are associated with any condition that involves activation of the coagulation pathway, including stroke, surgery, cancer, disseminated intravascular coagulation, nephrosis, and thrombotic thrombocytopenic purpura. FPA is released into the circulation following thrombin activation and cleavage of fibrinogen. Because FPA is a small polypeptide, it is likely cleared from the bloodstream rapidly. FPA has been demonstrated to be elevated for more than one month following clot formation, and maximum plasma FPA concentrations can exceed 40 ng/ml in active angina (Gensini, G. F. et al., Thromb. Res. 50:517-525, 1988; Tohgi, H. et al., Stroke 21:1663-1667, 1990).

Platelet-derived growth factor (PDGF) is a 28 kDa secreted homo- or heterodimeric protein composed of the homologous subunits A and/or B (Mahadevan, D. et al., J. Biol. Chem. 270:27595-27600, 1995). PDGF is a potent mitogen for mesenchymal cells, and has been implicated in the pathogenesis of atherosclerosis. PDGF is released by aggregating platelets and monocytes near sites of vascular injury. The normal plasma concentration of PDGF is <0.4 ng/ml (15 pM). Plasma PDGF concentrations are higher in individuals with acute myocardial infarction and unstable angina than in healthy controls or individuals with stable angina (Ogawa, H. et al., Am. J. Cardiol. 69:453-456, 1992; Wallace, J. M. et al., Ann. Clin. Biochem. 35:236-241, 1998; Ogawa, H. et al., Coron. Artery Dis. 4:437-442, 1993). Changes in the plasma PDGF concentration in these individuals is most likely due to increased platelet and monocyte activation. Plasma PDGF is elevated in individuals with brain tumors, breast cancer, and hypertension (Kurimoto, M. et al., Acta Neurochir. (Wien) 137:182-187, 1995; Seymour, L. et al., Breast Cancer Res. Treat. 26:247-252, 1993; Rossi, E. et al., Am. J. Hypertens. 11:1239-1243, 1998). Plasma PDGF may also be elevated in any pro-inflammatory condition or any condition that causes platelet activation including surgery, trauma, disseminated intravascular coagulation, and thrombotic thrombocytopenic purpura. PDGF is released from the secretory granules of platelets and monocytes upon activation. PDGF has a biphasic half-life of approximately 5 minutes and 1 hour in animals (Cohen, A. M. et al., J. Surg. Res. 49:447-452, 1990; Bowen-Pope, D. F. et al., Blood 64:458-469, 1984). The plasma PDGF concentration in ACS can exceed 0.6 ng/ml (22 pM) (Ogawa, H. et al., Am. J. Cardiol. 69:453-456, 1992). PDGF may be a sensitive and specific marker of platelet activation. In addition, it may be a sensitive marker of vascular injury, and the accompanying monocyte and platelet activation.

Prothrombin fragment 1+2 is a 32 kDa polypeptide that is liberated from the amino terminus of thrombin during thrombin activation. The normal plasma concentration of F1+2 is <32 ng/ml (1 nM). The plasma concentration of F1+2 is reportedly elevated in patients with acute myocardial infarction and unstable angina, but not stable angina, but the changes were not robust (Merlini, P. A. et al., Circulation 90:61-68, 1994). Other reports have indicated that there is no significant change in the plasma F1+2 concentration in cardiovascular disease (Biasucci, L. M. et al., Circulation 93:2121-2127, 1996; Manten, A. et al., Cardiovasc. Res. 40:389-395, 1998). The concentration of F1+2 in plasma can be elevated during any condition associated with coagulation activation, including stroke, surgery, trauma, thrombotic thrombocytopenic purpura, and disseminated intravascular coagulation. F1+2 is released into the bloodstream immediately upon thrombin activation. F1+2 has a half-life of approximately 90 minutes in plasma, and it has been suggested that this long half-life may mask bursts of thrombin formation (Biasucci, L. M. et al., Circulation 93:2121-2127, 1996).

P-selectin, also called granule membrane protein-140, GMP-140, PADGEM, and CD-62P, is a ˜140 kDa adhesion molecule expressed in platelets and endothelial cells. P-selectin is stored in the alpha granules of platelets and in the Weibel-Palade bodies of endothelial cells. Upon activation, P-selectin is rapidly translocated to the surface of endothelial cells and platelets to facilitate the “rolling” cell surface interaction with neutrophils and monocytes. Membrane-bound and soluble forms of P-selectin have been identified. Soluble P-selectin may be produced by shedding of membrane-bound P-selectin, either by proteolysis of the extracellular P-selectin molecule, or by proteolysis of components of the intracellular cytoskeleton in close proximity to the surface-bound P-selectin molecule (Fox, J. E., Blood Coagul. Fibrinolysis 5:291-304, 1994). Additionally, soluble P-selectin may be translated from mRNA that does not encode the N-terminal transmembrane domain (Dunlop, L. C. et al., J. Exp. Med. 175:1147-1150, 1992; Johnston, G. I. et al., J. Biol. Chem. 265:21381-21385, 1990). Activated platelets can shed membrane-bound P-selectin and remain in the circulation, and the shedding of P-selectin can elevate the plasma P-selectin concentration by approximately 70 ng/ml (Michelson, A. D. et al., Proc. Natl. Acad. Sci. U.S.A. 93:11877-11882, 1996). Soluble P-selectin may also adopt a different conformation than membrane-bound P-selectin. Soluble P-selectin has a monomeric rod-like structure with a globular domain at one end, and the membrane-bound molecule forms rosette structures with the globular domain facing outward (Ushiyama, S. et al., J. Biol. Chem. 268:15229-15237, 1993). Soluble P-selectin may play an important role in regulating inflammation and thrombosis by blocking interactions between leukocytes and activated platelets and endothelial cells (Gamble, J. R. et al., Science 249:414-417, 1990). The normal plasma concentration of soluble P-selectin is <200 ng/ml. Blood is normally collected using citrate as an anticoagulant, but some studies have used EDTA plasma with additives such as prostaglandin E to prevent platelet activation. EDTA may be a suitable anticoagulant that will yield results comparable to those obtained using citrate. Furthermore, the plasma concentration of soluble P-selectin may not be affected by potential platelet activation during the sampling procedure. The plasma soluble P-selectin concentration was significantly elevated in patients with acute myocardial infarction and unstable angina, but not stable angina, even following an exercise stress test (Ikeda, H. et al., Circulation 92:1693-1696, 1995; Tomoda, H. and Aoki, N., Angiology 49:807-813, 1998; Hollander, J. E. et al., J. Am. Coll. Cardiol. 34:95-105, 1999; Kaikita, K. et al., Circulation 92:1726-1730, 1995; Ikeda, H. et al., Coron. Artery. Dis. 5:515-518, 1994). The sensitivity and specificity of membrane-bound P-selectin versus soluble P-selectin for acute myocardial infarction is 71% versus 76% and 32% versus 45% (Hollander, J. E. et al., J. Am. Coll. Cardiol. 34:95-105, 1999). The sensitivity and specificity of membrane-bound P-selectin versus soluble P-selectin for unstable angina+acute myocardial infarction is 71% versus 79% and 30% versus 35% (Hollander, J. E. et al., J. Am. Coll. Cardiol. 34:95-105, 1999). P-selectin expression is greater in coronary atherectomy specimens from individuals with unstable angina than stable angina (Tenaglia, A. N. et al., Am. J. Cardiol. 79:742-747, 1997). Furthermore, plasma soluble P-selectin may be elevated to a greater degree in patients with acute myocardial infarction than in patients with unstable angina. Plasma soluble and membrane-bound P-selectin also is elevated in individuals with non-insulin dependent diabetes mellitus and congestive heart failure (Nomura, S. et al., Thromb. Haemost. 80:388-392, 1998; O'Connor, C. M. et al., Am. J. Cardiol. 83:1345-1349, 1999). Soluble P-selectin concentration is elevated in the plasma of individuals with idiopathic thrombocytopenic purpura, rheumatoid arthritis, hypercholesterolemia, acute stroke, atherosclerosis, hypertension, acute lung injury, connective tissue disease, thrombotic thrombocytopenic purpura, hemolytic uremic syndrome, disseminated intravascular coagulation, and chronic renal failure (Katayama, M. et al., Br. J. Haematol. 84:702-710, 1993; Haznedaroglu, I. C. et al., Acta Haematol. 101:16-20, 1999; Ertenli, I. et al., J. Rheumatol. 25:1054-1058, 1998; Davi, G. et al., Circulation 97:953-957, 1998; Frijns, C. J. et al., Stroke 28:2214-2218, 1997; Blann, A. D. et al., Thromb. Haemost. 77:1077-1080, 1997; Blann, A. D. et al., J. Hum. Hypertens. 11:607-609, 1997; Sakamaki, F. et al., A. J. Respir. Crit. Care Med. 151:1821-1826, 1995; Takeda, I. et al., Int. Arch. Allergy Immunol. 105:128-134, 1994; Chong, B. H. et al., Blood 83:1535-1541, 1994; Bonomini, M. et al., Nephron 79:399-407, 1998). Additionally, any condition that involves platelet activation can potentially be a source of plasma elevations in P-selectin. P-selectin is rapidly presented on the cell surface following platelet of endothelial cell activation. Soluble P-selectin that has been translated from an alternative mRNA lacking a transmembrane domain is also released into the extracellular space following this activation. Soluble P-selectin can also be formed by proteolysis involving membrane-bound P-selectin, either directly or indirectly. Plasma soluble P-selectin is elevated on admission in patients with acute myocardial infarction treated with tPA or coronary angioplasty, with a peak elevation occurring 4 hours after onset (Shimomura, H. et al., Am. J. Cardiol. 81:397-400, 1998). Plasma soluble P-selectin was elevated less than one hour following an anginal attack in patients with unstable angina, and the concentration decreased with time, approaching baseline more than 5 hours after attack onset (Ikeda, H. et al., Circulation 92:1693-1696, 1995). The plasma concentration of soluble P-selectin can approach 1 μg/ml in ACS (Ikeda, H. et al., Coron. Artery Dis. 5:515-518, 1994). Further investigation into the release of soluble P-selectin into and its removal from the bloodstream need to be conducted. P-selectin may be a sensitive and specific marker of platelet and endothelial cell activation, conditions that support thrombus formation and inflammation. It is not, however, a specific marker of ACS. When used with another marker that is specific for cardiac tissue injury, P-selectin may be useful in the discrimination of unstable angina and acute myocardial infarction from stable angina. Furthermore, soluble P-selectin may be elevated to a greater degree in acute myocardial infarction than in unstable angina. P-selectin normally exists in two forms, membrane-bound and soluble. Published investigations note that a soluble form of P-selectin is produced by platelets and endothelial cells, and by shedding of membrane-bound P-selectin, potentially through a proteolytic mechanism. Soluble P-selectin may prove to be the most useful currently identified marker of platelet activation, since its plasma concentration may not be as influenced by the blood sampling procedure as other markers of platelet activation, such as PF4 and β-TG.

Thrombin is a 37 kDa serine proteinase that proteolytically cleaves fibrinogen to form fibrin, which is ultimately integrated into a crosslinked network during clot formation. Antithrombin III (ATIII) is a 65 kDa serine proteinase inhibitor that is a physiological regulator of thrombin, factor XIa, factor XIIa, and factor IXa proteolytic activity. The inhibitory activity of ATIII is dependent upon the binding of heparin. Heparin enhances the inhibitory activity of ATIII by 2-3 orders of magnitude, resulting in almost instantaneous inactivation of proteinases inhibited by ATIII. ATIII inhibits its target proteinases through the formation of a covalent 1:1 stoichiometric complex. The normal plasma concentration of the approximately 100 kDa thrombin-ATIII complex (TAT) is <5 ng/ml (50 pM). TAT concentration is elevated in patients with acute myocardial infarction and unstable angina, especially during spontaneous ischemic episodes (Biasucci, L. M. et al., Am. J. Cardiol. 77:85-87, 1996; Kienast, J. et al., Thromb. Haemost. 70:550-553, 1993). Furthermore, TAT may be elevated in the plasma of individuals with stable angina (Manten, A. et al., Cardiovasc. Res. 40:389-395, 1998). Other published reports have found no significant differences in the concentration of TAT in the plasma of patients with ACS (Manten, A. et al., Cardiovasc. Res. 40:389-395, 1998; Hoffmeister, H. M. et al., Atherosclerosis 144:151-157, 1999). Further investigation is needed to determine plasma TAT concentration changes associated with ACS. Elevation of the plasma TAT concentration is associated with any condition associated with coagulation activation, including stroke, surgery, trauma, disseminated intravascular coagulation, and thrombotic thrombocytopenic purpura. TAT is formed immediately following thrombin activation in the presence of heparin, which is the limiting factor in this interaction. TAT has a half-life of approximately 5 minutes in the bloodstream (Biasucci, L. M. et al., Am. J. Cardiol. 77:85-87, 1996). TAT concentration is elevated in, exhibits a sharp drop after 15 minutes, and returns to baseline less than 1 hour following coagulation activation. The plasma concentration of TAT can approach 50 ng/ml in ACS (Biasucci, L. M. et al., Circulation 93:2121-2127, 1996). TAT is a specific marker of coagulation activation, specifically, thrombin activation.

von Willebrand factor (vWF) is a plasma protein produced by platelets, megakaryocytes, and endothelial cells composed of 220 kDa monomers that associate to form a series of high molecular weight multimers. These multimers normally range in molecular weight from 600-20,000 kDa. vWF participates in the coagulation process by stabilizing circulating coagulation factor VIII and by mediating platelet adhesion to exposed subendothelium, as well as to other platelets. The A1 domain of vWF binds to the platelet glycoprotein Ib-IX-V complex and non-fibrillar collagen type VI, and the A3 domain binds fibrillar collagen types I and III (Emsley, J. et al., J. Biol. Chem. 273:10396-10401, 1998). Other domains present in the vWF molecule include the integrin binding domain, which mediates platelet-platelet interactions, the the protease cleavage domain, which appears to be relevant to the pathogenesis of type 11A von Willebrand disease. The interaction of vWF with platelets is tightly regulated to avoid interactions between vWF and platelets in normal physiologic conditions. vWF normally exists in a globular state, and it undergoes a conformation transition to an extended chain structure under conditions of high sheer stress, commonly found at sites of vascular injury. This conformational change exposes intramolecular domains of the molecule and allows vWF to interact with platelets. Furthermore, shear stress may cause vWF release from endothelial cells, making a larger number of vWF molecules available for interactions with platelets. The conformational change in vWF can be induced in vitro by the addition of non-physiological modulators like ristocetin and botrocetin (Miyata, S. et al., J. Biol. Chem. 271:9046-9053, 1996). At sites of vascular injury, vWF rapidly associates with collagen in the subendothelial matrix, and virtually irreversibly binds platelets, effectively forming a bridge between platelets and the vascular subendothelium at the site of injury. Evidence also suggests that a conformational change in vWF may not be required for its interaction with the subendothelial matrix (Sixma, J. J. and de Groot, P. G., Mayo Clin. Proc. 66:628-633, 1991). This suggests that vWF may bind to the exposed subendothelial matrix at sites of vascular injury, undergo a conformational change because of the high localized shear stress, and rapidly bind circulating platelets, which will be integrated into the newly formed thrombus. Measurement of the total amount of vWF would allow one who is skilled in the art to identify changes in total vWF concentration associated with stroke or cardiovascular disease. This measurement could be performed through the measurement of various forms of the vWF molecule. Measurement of the A1 domain would allow the measurement of active vWF in the circulation, indicating that a pro-coagulant state exists because the A1 domain is accessible for platelet binding. In this regard, an assay that specifically measures vWF molecules with both the exposed A1 domain and either the integrin binding domain or the A3 domain would also allow for the identification of active vWF that would be available for mediating platelet-platelet interactions or mediate crosslinking of platelets to vascular subendothelium, respectively. Measurement of any of these vWF forms, when used in an assay that employs antibodies specific for the protease cleavage domain may allow assays to be used to determine the circulating concentration of various vWF forms in any individual, regardless of the presence of von Willebrand disease. The normal plasma concentration of vWF is 5-10 μg/ml, or 60-110% activity, as measured by platelet aggregation. The measurement of specific forms of vWF may be of importance in any type of vascular disease, including stroke and cardiovascular disease. The plasma vWF concentration is reportedly elevated in individuals with acute myocardial infarction and unstable angina, but not stable angina (Goto, S. et al., Circulation 99:608-613, 1999; Tousoulis, D. et al., Int. J. Cardiol. 56:259-262, 1996; Yazdani, S. et al., J Am Coll Cardiol 30:1284-1287, 1997; Montalescot, G. et al., Circulation 98:294-299). Furthermore, elevations of the plasma vWF concentration may be a predictor of adverse clinical outcome in patients with unstable angina (Montalescot, G. et al., Circulation 98:294-299). vWF concentrations also have been demonstrated to be elevated in patients with stroke and subarachnoid hemorrhage, and also appear to be useful in assessing risk of mortality following stroke (Blann, A. et al., Blood Coagul. Fibrinolysis 10:277-284, 1999; Hirashima, Y. et al. Neurochem Res. 22:1249-1255, 1997; Catto, A. J. et al., Thromb. Hemost. 77:1104-1108, 1997). The plasma concentration of vWF may be elevated in conjunction with any event that is associated with endothelial cell damage or platelet activation. vWF is present at high concentration in the bloodstream, and it is released from platelets and endothelial cells upon activation. vWF would likely have the greatest utility as a marker of platelet activation or, specifically, conditions that favor platelet activation and adhesion to sites of vascular injury. The conformation of vWF is also known to be altered by high shear stress, as would be associated with a partially stenosed blood vessel. As the blood flows past a stenosed vessel, it is subjected to shear stress considerably higher than is encountered in the circulation of an undiseased individual.

Tissue factor (TF) is a 45 kDa cell surface protein expressed in brain, kidney, and heart, and in a transcriptionally regulated manner on perivascular cells and monocytes. TF forms a complex with factor VIIa in the presence of Ca²⁺ ions, and it is physiologically active when it is membrane bound. This complex proteolytically cleaves factor X to form factor Xa. It is normally sequestered from the bloodstream. Tissue factor can be detected in the bloodstream in a soluble form, bound to factor VIIa, or in a complex with factor VIIa, and tissue factor pathway inhibitor that can also include factor Xa. TF also is expressed on the surface of macrophages, which are commonly found in atherosclerotic plaques. The normal serum concentration of TF is <0.2 ng/ml (4.5 pM). The plasma TF concentration is elevated in patients with ischemic heart disease (Falciani, M. et al., Thromb. Haemost. 79:495-499, 1998). TF is elevated in patients with unstable angina and acute myocardial infarction, but not in patients with stable angina (Falciani, M. et al., Thromb. Haemost. 79:495-499, 1998; Suefuji, H. et al., Am. Heart J. 134:253-259, 1997; Misumi, K. et al., Am. J. Cardiol. 81:22-26, 1998). Furthermore, TF expression on macrophages and TF activity in atherosclerotic plaques is more common in unstable angina than stable angina (Soejima, H. et al., Circulation 99:2908-2913, 1999; Kaikita, K. et al., Arterioscler. Thromb. Vasc. Biol. 17:2232-2237, 1997; Ardissino, D. et al., Lancet 349:769-771, 1997). The differences in plasma TF concentration in stable versus unstable angina may not be of statistical significance. Elevations in the serum concentration of TF are associated with any condition that causes or is a result of coagulation activation through the extrinsic pathway. These conditions can include subarachnoid hemorrhage, disseminated intravascular coagulation, renal failure, vasculitis, and sickle cell disease. (Hirashima, Y. et al., Stroke 28:1666-1670, 1997; Takahashi, H. et al., Am. J. Hematol. 46:333-337, 1994; Koyama, T. et al., Br. J. Haematol. 87:343-347, 1994). TF is released immediately when vascular injury is coupled with extravascular cell injury. TF levels in ischemic heart disease patients can exceed 800 pg/ml within 2 days of onset (Falciani, M. et al., Thromb. Haemost. 79:495-499, 1998. TF levels were decreased in the chronic phase of acute myocardial infarction, as compared with the chronic phase (Suefuji, H. et al., Am. Heart J 134:253-259, 1997). TF is a specific marker for activation of the extrinsic coagulation pathway and the presence of a general hypercoagulable state. It may be a sensitive marker of vascular injury resulting from plaque rupture

The coagulation cascade can be activated through either the extrinsic or intrinsic pathways. These enzymatic pathways share one final common pathway. The first step of the common pathway involves the proteolytic cleavage of prothrombin by the factor Xa/factor Va prothrombinase complex to yield active thrombin. Thrombin is a serine proteinase that proteolytically cleaves fibrinogen. Thrombin first removes fibrinopeptide A from fibrinogen, yielding desAA fibrin monomer, which can form complexes with all other fibrinogen-derived proteins, including fibrin degradation products, fibrinogen degradation products, desAA fibrin, and fibrinogen. The desAA fibrin monomer is generically referred to as soluble fibrin, as it is the first product of fibrinogen cleavage, but it is not yet crosslinked via factor XIIIa into an insoluble fibrin clot. DesAA fibrin monomer also can undergo further proteolytic cleavage by thrombin to remove fibrinopeptide B, yielding desAABB fibrin monomer. This monomer can polymerize with other desAABB fibrin monomers to form soluble desAABB fibrin polymer, also referred to as soluble fibrin or thrombus precursor protein (TpP™). TpP™ is the immediate precursor to insoluble fibrin, which forms a “mesh-like” structure to provide structural rigidity to the newly formed thrombus. In this regard, measurement of TpP™ in plasma is a direct measurement of active clot formation. The normal plasma concentration of TpP™ is <6 ng/ml (Laurino, J. P. et al., Ann. Clin. Lab. Sci. 27:338-345, 1997). American Biogenetic Sciences has developed an assay for TpP™ (U.S. Pat. Nos. 5,453,359 and 5,843,690) and states that its TpP™ assay can assist in the early diagnosis of acute myocardial infarction, the ruling out of acute myocardial infarction in chest pain patients, and the identification of patients with unstable angina that will progress to acute myocardial infarction. Other studies have confirmed that TpP™ is elevated in patients with acute myocardial infarction, most often within 6 hours of onset (Laurino, J. P. et al., Ann. Clin. Lab. Sci. 27:338-345, 1997; Carville, D. G. et al., Clin. Chem. 42:1537-1541, 1996). The plasma concentration of TpP™ is also elevated in patients with unstable angina, but these elevations may be indicative of the severity of angina and the eventual progression to acute myocardial infarction (Laurino, J. P. et al., Ann. Clin. Lab. Sci. 27:338-345, 1997). The concentration of TpP™ in plasma will theoretically be elevated during any condition that causes or is a result of coagulation activation, including disseminated intravascular coagulation, deep venous thrombosis, congestive heart failure, surgery, cancer, gastroenteritis, and cocaine overdose (Laurino, J. P. et al., Ann. Clin. Lab. Sci. 27:338-345, 1997). TpP™ is released into the bloodstream immediately following thrombin activation. TpP™ likely has a short half-life in the bloodstream because it will be rapidly converted to insoluble fibrin at the site of clot formation. Plasma TpP™ concentrations peak within 3 hours of acute myocardial infarction onset, returning to normal after 12 hours from onset. The plasma concentration of TpP™ can exceed 30 ng/ml in CVD (Laurino, J. P. et al., Ann. Clin. Lab. Sci. 27:338-345, 1997). TpP™ is a sensitive and specific marker of coagulation activation. It has been demonstrated that TpP™ is useful in the diagnosis of acute myocardial infarction, but only when it is used in conjunction with a specific marker of cardiac tissue injury.

(iii) Exemplar Markers Related to Atherosclerotic Plague Rupture

The appearance of markers related to atherosclerotic plaque rupture may preceed specific markers of myocardial injury. Potential markers of atherosclerotic plaque rupture include human neutrophil elastase, inducible nitric oxide synthase, lysophosphatidic acid, malondialdehyde-modified low density lipoprotein, and various members of the matrix metalloproteinase (MMP) family, including MMP-1, -2, -3, and -9.

Human neutrophil elastase (HNE) is a 30 kDa serine proteinase that is normally contained within the azurophilic granules of neutrophils. HNE is released upon neutrophil activation, and its activity is regulated by circulating α₁-proteinase inhibitor. Activated neutrophils are commonly found in atherosclerotic plaques, and rupture of these plaques may result in the release of HNE. The plasma HNE concentration is usually measured by detecting HNE-α₁-PI complexes. The normal concentration of these complexes is 50 ng/ml, which indicates a normal concentration of approximately 25 ng/ml (0.8 nM) for HNE. HNE release also can be measured through the specific detection of fibrinopeptide Bβ₃₀₋₄₃, a specific HNE-derived fibrinopeptide, in plasma. Plasma HNE is elevated in patients with coronary stenosis, and its elevation is greater in patients with complex plaques than those with simple plaques (Kosar, F. et al., Angiology 49:193-201, 1998; Amaro, A. et al., Eur. Heart J 16:615-622, 1995). Plasma HNE is not significantly elevated in patients with stable angina, but is elevated inpatients with unstable angina and acute myocardial infarction, as determined by measuring fibrinopeptide Bβ₃₀₋₄₃, with concentrations in unstable angina being 2.5-fold higher than those associated with acute myocardial infarction (Dinerman, J. L. et al., J. Am. Coll. Cardiol. 15:1559-1563, 1990; Mehta, J. et al., Circulation 79:549-556, 1989). Serum HNE is elevated in cardiac surgery, exercise-induced muscle damage, giant cell arteritis, acute respiratory distress syndrome, appendicitis, pancreatitis, sepsis, smoking-associated emphysema, and cystic fibrosis (Genereau, T. et al., J. Rheumatol. 25:710-713, 1998; Mooser, V. et al., Arterioscler. Thromb. Vasc. Biol. 19:1060-1065, 1999; Gleeson, M. et al. Eur. J. Appl. Physiol. 77:543-546, 1998; Gando, S. et al., J Trauma 42:1068-1072, 1997; Eriksson, S. et al., Eur. J. Surg. 161:901-905, 1995; Liras, G. et al., Rev. Esp. Enferm. Dig. 87:641-652, 1995; Endo, S. et al., J Inflamm. 45:136-142, 1995; Janoff, A., Annu Rev Med 36:207-216, 1985). HNE may also be released during blood coagulation (Plow, E. F. and Plescia, J., Thromb. Haemost. 59:360-363, 1988; Plow, E. F., J. Clin. Invest. 69:564-572, 1982). Serum elevations of HNE could also be associated with any non-specific infection or inflammatory state that involves neutrophil recruitment and activation. It is most likely released upon plaque rupture, since activated neutrophils are present in atherosclerotic plaques. HNE is presumably cleared by the liver after it has formed a complex with α₁-PI.

Inducible nitric oxide synthase (iNOS) is a 130 kDa cytosolic protein in epithelial cells macrophages whose expression is regulated by cytokines, including interferon-γ, interleukin-1β, interleukin-6, and tumor necrosis factor a, and lipopolysaccharide. iNOS catalyzes the synthesis of nitric oxide (NO) from L-arginine, and its induction results in a sustained high-output production of NO, which has antimicrobial activity and is a mediator of a variety of physiological and inflammatory events. NO production by iNOS is approximately 100 fold more than the amount produced by constitutively-expressed NOS (Depre, C. et al., Cardiovasc. Res. 41:465-472, 1999). There are no published investigations of plasma iNOS concentration changes associated with ACS. iNOS is expressed in coronary atherosclerotic plaque, and it may interfere with plaque stability through the production of peroxynitrate, which is a product of NO and superoxide and enhances platelet adhesion and aggregation (Depre, C. et al., Cardiovasc. Res. 41:465-472, 1999). iNOS expression during myocardial ischemia may not be elevated, suggesting that iNOS may be useful in the differentiation of angina from acute myocardial infarction (Hammerman, S. I. et al., Am. J. Physiol. 277:H1579-H1592, 1999; Kaye, D. M. et al., Life Sci 62:883-887, 1998). Elevations in the plasma iNOS concentration may be associated with cirrhosis, iron-deficiency anemia, or any other condition that results in macrophage activation, including bacterial infection (Jimenez, W. et al., Hepatology 30:670-676, 1999; Ni, Z. et al., Kidney Int. 52:195-201, 1997). iNOS may be released into the bloodstream as a result of atherosclerotic plaque rupture, and the presence of increased amounts of iNOS in the bloodstream may not only indicate that plaque rupture has occurred, but also that an ideal environment has been created to promote platelet adhesion. However, iNOS is not specific for atherosclerotic plaque rupture, and its expression can be induced during non-specific inflammatory conditions.

Lysophosphatidic acid (LPA) is a lysophospholipid intermediate formed in the synthesis of phosphoglycerides and triacylglycerols. It is formed by the acylation of glycerol-3 phosphate by acyl-coenzyme A and during mild oxidation of low-density lipoprotein (LDL). LPA is a lipid second messanger with vasoactive properties, and it can function as a platelet activator. LPA is a component of atherosclerotic lesions, particularly in the core, which is most prone to rupture (Siess, W., Proc. Natl. Acad. Sci. U.S.A. 96, 6931-6936, 1999). The normal plasma LPA concentration is 540 nM. Serum LPA is elevated in renal failure and in ovarian cancer and other gynecologic cancers (Sasagawa, T. et al., J. Nutr. Sci. Vitaminol. (Tokyo) 44:809-818, 1998; Xu, Y. et al., JAMA 280:719-723, 1998). In the context of unstable angina, LPA is most likely released as a direct result of plaque rupture. The plasma LPA concentration can exceed 60 μM in patients with gynecologic cancers (Xu, Y. et al., JAMA 280:719-723, 1998). Serum LPA may be a useful marker of atherosclerotic plaque rupture.

Malondialdehyde-modified low-density lipoprotein (MDA-modified LDL) is formed during the oxidation of the apoB-100 moiety of LDL as a result of phospholipase activity, prostaglandin synthesis, or platelet activation. MDA-modified LDL can be distinguished from oxidized LDL because MDA modifications of LDL occur in the absence of lipid peroxidation (Holvoet, P., Acta Cardiol. 53:253-260, 1998). The normal plasma concentration of MDA-modified LDL is less than 4 μg/ml (˜10 μM). Plasma concentrations of oxidized LDL are elevated in stable angina, unstable angina, and acute myocardial infarction, indicating that it may be a marker of atherosclerosis (Holvoet, P., Acta Cardiol. 53:253-260, 1998; Holvoet, P. et al., Circulation 98:1487-1494, 1998). Plasma MDA-modified LDL is not elevated in stable angina, but is significantly elevated in unstable angina and acute myocardial infarction (Holvoet, P., Acta Cardiol. 53:253-260, 1998; Holvoet, P. et al., Circulation 98:1487-1494, 1998; Holvoet, P. et al., JAMA 281:1718-1721, 1999). Plasma MDA-modified LDL is elevated in individuals with beta-thallasemia and in renal transplant patients (Livrea, M. A. et al., Blood 92:3936-3942, 1998; Ghanem, H. et al., Kidney Int. 49:488-493, 1996; van den Dorpel, M. A. et al., Transpl. Int. 9 Suppl. 1:S54-S57, 1996). Furthermore, serum MDA-modified LDL may be elevated during hypoxia (Balagopalakrishna, C. et al., Adv. Exp. Med. Biol. 411:337-345, 1997). The plasma concentration of MDA-modified LDL is elevated within 6-8 hours from the onset of chest pain. Plasma concentrations of MDA-modified LDL can approach 20 μg/ml (˜50 μM) in patients with acute myocardial infarction, and 15 μg/ml (˜40 μM) in patients with unstable angina (Holvoet, P. et al., Circulation 98:1487-1494, 1998). Plasma MDA-modified LDL has a half-life of less than 5 minutes in mice (Ling, W. et al., J. Clin. Invest. 100:244-252, 1997). MDA-modified LDL appears to be a specific marker of atherosclerotic plaque rupture in acute coronary symptoms. It is unclear, however, if elevations in the plasma concentration of MDA-modified LDL are a result of plaque rupture or platelet activation. The most reasonable explanation is that the presence of increased amounts of MDA-modified LDL is an indication of both events. MDA-modified LDL may be useful in discriminating unstable angina and acute myocardial infarction from stable angina.

Matrix metalloproteinase-1 (MMP-1), also called collagenase-1, is a 41/44 kDa zinc- and calcium-binding proteinase that cleaves primarily type I collagen, but can also cleave collagen types II, III, VII and X. The active 41/44 kDa enzyme can undergo autolysis to the still active 22/27 kDa form. MMP-1 is synthesized by a variety of cells, including smooth muscle cells, mast cells, macrophage-derived foam cells, T lymphocytes, and endothelial cells (Johnson, J. L. et al., Arterioscler. Thromb. Vasc. Biol. 18:1707-1715, 1998). MMP-1, like other MMPs, is involved in extracellular matrix remodeling, which can occur following injury or during intervascular cell migration. MMP-1 can be found in the bloodstream either in a free form or in complex with TIMP-1, its natural inhibitor. MMP-1 is normally found at a concentration of <25 ng/ml in plasma. MMP-1 is found in the shoulder region of atherosclerotic plaques, which is the region most prone to rupture, and may be involved in atherosclerotic plaque destabilization (Johnson, J. L. et al., Arterioscler. Thromb. Vasc. Biol. 18:1707-1715, 1998). Furthermore, MMP-1 has been implicated in the pathogenesis of myocardial reperfusion injury (Shibata, M. et al., Angiology 50:573-582, 1999). Serum MMP-1 may be elevated inflammatory conditions that induce mast cell degranulation. Serum MMP-1 concentrations are elevated in patients with arthritis and systemic lupus erythematosus (Keyszer, G. et al., Z Rheumatol 57:392-398, 1998; Keyszer, G. J. Rheumatol. 26:251-258, 1999). Serum MMP-1 also is elevated in patients with prostate cancer, and the degree of elevation corresponds to the metastatic potential of the tumor (Baker, T. et al., Br. J. Cancer 70:506-512, 1994). The serum concentration of MMP-1 may also be elevated in patients with other types of cancer. Serum MMP-1 is decreased in patients with hemochromatosis and also in patients with chronic viral hepatitis, where the concentration is inversely related to the severity (George, D. K. et al., Gut 42:715-720, 1998; Murawaki, Y. et al., J. Gastroenterol. Hepatol. 14:138-145, 1999). Serum MMP-1 was decreased in the first four days following acute myocardial infarction, and increased thereafter, reaching peak levels 2 weeks after the onset of acute myocardial infarction (George, D. K. et al., Gut 42:715-720, 1998).

Matrix metalloproteinase-2 (MMP-2), also called gelatinase A, is a 66 kDa zinc- and calcium-binding proteinase that is synthesized as an inactive 72 kDa precursor. Mature MMP-3 cleaves type I gelatin and collagen of types IV, V, VII, and X. MMP-2 is synthesized by a variety of cells, including vascular smooth muscle cells, mast cells, macrophage-derived foam cells, T lymphocytes, and endothelial cells (Johnson, J. L. et al., Arterioscler. Thromb. Vasc. Biol. 18:1707-1715, 1998). MMP-2 is usually found in plasma in complex with TIMP-2, its physiological regulator (Murawaki, Y. et al., J. Hepatol. 30:1090-1098, 1999). The normal plasma concentration of MMP-2 is <˜550 ng/ml (8 nM). MMP-2 expression is elevated in vascular smooth muscle cells within atherosclerotic lesions, and it may be released into the bloodstream in cases of plaque instability (Kai, H. et al., J. Am. Coll. Cardiol. 32:368-372, 1998). Furthermore, MMP-2 has been implicated as a contributor to plaque instability and rupture (Shah, P. K. et al., Circulation 92:1565-1569, 1995). Serum MMP-2 concentrations were elevated in patients with stable angina, unstable angina, and acute myocardial infarction, with elevations being significantly greater in unstable angina and acute myocardial infarction than in stable angina (Kai, H. et al., J. Am. Coll. Cardiol. 32:368-372, 1998). There was no change in the serum MMP-2 concentration in individuals with stable angina following a treadmill exercise test (Kai, H. et al., J. Am. Coll. Cardiol. 32:368-372, 1998). Serum and plasma MMP-2 is elevated in patients with gastric cancer, hepatocellular carcinoma, liver cirrhosis, urothelial carcinoma, rheumatoid arthritis, and lung cancer (Murawaki, Y. et al., J. Hepatol. 30:1090-1098, 1999; Endo, K. et al., Anticancer Res. 17:2253-2258, 1997; Gohji, K. et al., Cancer 78:2379-2387, 1996; Gruber, B. L. et al., Clin. Immunol. Immunopathol. 78:161-171, 1996; Garbisa, S. et al., Cancer Res. 52:4548-4549, 1992). Furthermore, MMP-2 may also be translocated from the platelet cytosol to the extracellular space during platelet aggregation (Sawicki, G. et al., Thromb. Haemost. 80:836-839, 1998). MMP-2 was elevated on admission in the serum of individuals with unstable angina and acute myocardial infarction, with maximum levels approaching 1.5 μg/ml (25 nM) (Kai, H. et al., J. Am. Coll. Cardiol. 32:368-372, 1998). The serum MMP-2 concentration peaked 1-3 days after onset in both unstable angina and acute myocardial infarction, and started to return to normal after 1 week (Kai, H. et al., J. Am. Coll. Cardiol. 32:368-372, 1998).

Matrix metalloproteinase-3 (MMP-3), also called stromelysin-1, is a 45 kDa zinc- and calcium-binding proteinase that is synthesized as an inactive 60 kDa precursor. Mature MMP-3 cleaves proteoglycan, fibrinectin, laminin, and type IV collagen, but not type I collagen. MMP-3 is synthesized by a variety of cells, including smooth muscle cells, mast cells, macrophage-derived foam cells, T lymphocytes, and endothelial cells (Johnson, J. L. et al., Arterioscler. Thromb. Vasc. Biol. 18:1707-1715, 1998). MMP-3, like other MMPs, is involved in extracellular matrix remodeling, which can occur following injury or during intervascular cell migration. MMP-3 is normally found at a concentration of <125 ng/ml in plasma. The serum MMP-3 concentration also has been shown to increase with age, and the concentration in males is approximately 2 times higher in males than in females (Manicourt, D. H. et al., Arthritis Rheum. 37:1774-1783, 1994). MMP-3 is found in the shoulder region of atherosclerotic plaques, which is the region most prone to rupture, and may be involved in atherosclerotic plaque destabilization (Johnson, J. L. et al., Arterioscler. Thromb. Vasc. Biol. 18:1707-1715, 1998). Therefore, MMP-3 concentration may be elevated as a result of atherosclerotic plaque rupture in unstable angina. Serum MMP-3 may be elevated inflammatory conditions that induce mast cell degranulation. Serum MMP-3 concentrations are elevated in patients with arthritis and systemic lupus erythematosus (Zucker, S. et al. J. Rheumatol. 26:78-80, 1999; Keyszer, G. et al., Z Rheumatol. 57:392-398, 1998; Keyszer, G. et al. J. Rheumatol. 26:251-258, 1999). Serum MMP-3 also is elevated in patients with prostate and urothelial cancer, and also glomerulonephritis (Lein, M. et al., Urologe A 37:377-381, 1998; Gohji, K. et al., Cancer 78:2379-2387, 1996; Akiyama, K. et al., Res. Commun. Mol. Pathol. Pharmacol. 95:115-128, 1997). The serum concentration of MMP-3 may also be elevated in patients with other types of cancer. Serum MMP-3 is decreased in patients with hemochromatosis (George, D. K. et al., Gut 42:715-720, 1998).

Matrix metalloproteinase-9 (MMP-9) also called gelatinase B, is an 84 kDa zinc- and calcium-binding proteinase that is synthesized as an inactive 92 kDa precursor. Mature MMP-9 cleaves gelatin types I and V, and collagen types IV and V. MMP-9 exists as a monomer, a homodimer, and a heterodimer with a 25 kDa α₂-microglobulin-related protein (Triebel, S. et al., FEBS Lett. 314:386-388, 1992). MMP-9 is synthesized by a variety of cell types, most notably by neutrophils. The normal plasma concentration of MMP-9 is <35 ng/ml (400 pM). MMP-9 expression is elevated in vascular smooth muscle cells within atherosclerotic lesions, and it may be released into the bloodstream in cases of plaque instability (Kai, H. et al., J. Am. Coll. Cardiol. 32:368-372, 1998). Furthermore, MMP-9 may have a pathogenic role in the development of ACS (Brown, D. L. et al., Circulation 91:2125-2131, 1995). Plasma MMP-9 concentrations are significantly elevated in patients with unstable angina and acute myocardial infarction, but not stable angina (Kai, H. et al., J. Am. Coll. Cardiol. 32:368-372, 1998). The elevations in patients with acute myocardial infarction may also indicate that those individuals were suffering from unstable angina. Elevations in the plasma concentration of MMP-9 may also be greater in unstable angina than in acute myocardial infarction. There was no significant change in plasma MMP-9 levels after a treadmill exercise test in patients with stable angina (Kai, H. et al., J. Am. Coll. Cardiol. 32:368-372, 1998). Plasma MMP-9 is elevated in individuals with rheumatoid arthritis, septic shock, giant cell arteritis and various carcinomas (Gruber, B. L. et al., Clin. Immunol. Immunopathol. 78:161-171, 1996; Nakamura, T. et al., Am. J. Med. Sci. 316:355-360, 1998; Blankaert, D. et al., J. Acquir. Immune Defic. Syndr. Hum. Retrovirol. 18:203-209, 1998; Endo, K. et al. Anticancer Res. 17:2253-2258, 1997; Hayasaka, A. et al., Hepatology 24:1058-1062, 1996; Moore, D. H. et al., Gynecol. Oncol. 65:78-82, 1997; Sorbi, D. et al., Arthritis Rheum. 39:1747-1753, 1996; lizasa, T. et al., Clin., Cancer Res. 5:149-153, 1999). Furthermore, the plasma MMP-9 concentration may be elevated in stroke and cerebral hemorrhage (Mun-Bryce, S. and Rosenberg, G. A., J. Cereb. Blood Flow Metab. 18:1163-1172, 1998; Romanic, A. M. et al., Stroke 29:1020-1030, 1998; Rosenberg, G. A., J. Neurotrauma 12:833-842, 1995). MMP-9 was elevated on admission in the serum of individuals with unstable angina and acute myocardial infarction, with maximum levels approaching 150 ng/ml (1.7 nM) (Kai, H. et al., J. Am. Coll. Cardiol. 32:368-372, 1998). The serum MMP-9 concentration was highest on admission in patients unstable angina, and the concentration decreased gradually after treatment, approaching baseline more than 1 week after onset (Kai, H. et al., J. Am. Coll. Cardiol. 32:368-372, 1998).

The balance between matrix metalloproteinases and their inhibitors is a critical factor which affects tumor invasion and metastasis. The TIMP family represents a class of small (21-28 kDa) related proteins that inhibit the metalloproteinases. Tissue inhibitor of metalloproteinase 1 (TIMP 1) is reportedly involved in the regulation of bone modeling and remodeling in normal developing human bone, involved in the invasive phenotype of acute myelogenous leukemia, demonstrating polymorphic X-chromosome inactivation. TIMP1 is known to act on mmp-1, mmp-2, mmp-3, mmp-7, mmp-8, mmp-9, mmp-10, mmp-11, mmp-12, mmp-13 and mmp-16. Tissue inhibitor of metalloproteinase 2 (TIMP2) complexes with metalloproteinases (such as collagenases) and irreversibly inactivates them. TIMP 2 is known to act on mmp-1, mmp-2, mmp-3, mmp-7, mmp-8, mmp-9, mmp-10, mmp-13, mmp-14, mmp-15, mmp-16 and mmp-19. Two alternatively spliced forms may be associated with SYN4, and involved in the invasive phenotype of acute myelogenous leukemia. Unlike the inducible expression of some other TIMP gene family members, the expression of this gene is largely constitutive. Tissue inhibitor of metalloproteinase 3 (TIMP3) antagonizes matrix metalloproteinase activity and can suppress tumor growth, angiogenesis, invasion, and metastasis. Loss of TIMP-3 has been related to the acquisition of tumorigenesis.

(iv) Exemplary Markers Related to Tissue Injury and Inflammation

Pulmonary surfactant protein D (SP-D) is a 43 kDa protein synthesized and secreted into the airspaces of the lung by the respiratory epithelium. At the alveolar level, SP-D is constitutively synthesized and secreted by alveolar type II cells. SP-D, a collagenous calcium-dependent lectin (or collectin), binds to surface glycoconjugates expressed by a wide variety of microorganisms, and to oligosaccharides associated with the surface of various complex organic antigens. SP-D also specifically interacts with glycoconjugates and other molecules expressed on the surface of macrophages, neutrophils, and lymphocytes. In addition, SP-D binds to specific surfactant-associated lipids and can influence the organization of lipid mixtures containing phosphatidylinositol in vitro. Consistent with these diverse in vitro activities is the observation that SP-D-deficient transgenic mice show abnormal accumulations of surfactant lipids, and respond abnormally to challenge with respiratory viruses and bacterial lipopolysaccharides. The phenotype of macrophages isolated from the lungs of SP-D-deficient mice is altered, and there is circumstantial evidence that abnormal oxidant metabolism and/or increased metalloproteinase expression contributes to the development of emphysema. The expression of SP-D is increased in response to many forms of lung injury, and deficient accumulation of appropriately oligomerized SP-D might contribute to the pathogenesis of a variety of human lung diseases. See, e.g., Crouch, Respir. Res. 1: 93-108 (2000).

C-reactive protein is a (CRP) is a homopentameric Ca²⁺-binding acute phase protein with 21 kDa subunits that is involved in host defense. CRP preferentially binds to phosphorylcholine, a common constituent of microbial membranes. Phosphorylcholine is also found in mammalian cell membranes, but it is not present in a form that is reactive with CRP. The interaction of CRP with phosphorylcholine promotes agglutination and opsonization of bacteria, as well as activation of the complement cascade, all of which are involved in bacterial clearance. Furthermore, CRP can interact with DNA and histones, and it has been suggested that CRP is a scavenger of nuclear material released from damaged cells into the circulation (Robey, F. A. et al., J. Biol. Chem. 259:7311-7316, 1984). CRP synthesis is induced by 11-6, and indirectly by IL-1, since IL-1 can trigger the synthesis of IL-6 by Kupffer cells in the hepatic sinusoids. The normal plasma concentration of CRP is <3 μg/ml (30 nM) in 90% of the healthy population, and <10 μg/ml (100 nM) in 99% of healthy individuals. Plasma CRP concentrations can be measured by rate nephelometry or ELISA. The plasma concentration of CRP is significantly elevated in patients with acute myocardial infarction and unstable angina, but not stable angina (Biasucci, L. M. et al., Circulation 94:874-877, 1996; Biasucci, L. M. et al., Am. J. Cardiol. 77:85-87, 1996; Benamer, H. et al., Am. J. Cardiol. 82:845-850, 1998; Caligiuri, G. et al., J. Am. Coll. Cardiol. 32:1295-1304, 1998; Curzen, N. P. et al., Heart 80:23-27, 1998; Dangas, G. et al., Am. J. Cardiol. 83:583-5, A7, 1999). CRP may also be elevated in the plasma of individuals with variant or resolving unstable angina, but mixed results have been reported (Benamer, H. et al., Am. J. Cardiol. 82:845-850, 1998; Caligiuri, G. et al., J. Am. Coll. Cardiol. 32:1295-1304, 1998). The concentration of CRP will be elevated in the plasma from individuals with any condition that may elicit an acute phase response, such as infection, surgery, trauma, and stroke. CRP is a secreted protein that is released into the bloodstream soon after synthesis. CRP synthesis is upregulated by IL-6, and the plasma CRP concentration is significantly elevated within 6 hours of stimulation (Biasucci, L. M. et al., Am. J. Cardiol. 77:85-87, 1996). The plasma CRP concentration peaks approximately 50 hours after stimulation, and begins to decrease with a half-life of approximately 19 hours in the bloodstream (Biasucci, L. M. et al., Am. J. Cardiol. 77:85-87, 1996). Other investigations have confirmed that the plasma CRP concentration in individuals with unstable angina (Biasucci, L. M. et al., Circulation 94:874-877, 1996). The plasma concentration of CRP can approach 100 μg/ml (1 μM) in individuals with ACS (Biasucci, L. M. et al., Circulation 94:874-877, 1996; Liuzzo, G. et al., Circulation 94:2373-2380, 1996). CRP is a specific marker of the acute phase response. Elevations of CRP have been identified in the plasma of individuals with acute myocardial infarction and unstable angina, most likely as a result of activation of the acute phase response associated with atherosclerotic plaque rupture or cardiac tissue injury.

Interleukins (ILs) are part of a larger class of polypeptides known as cytokines. These are messenger molecules that transmit signals between various cells of the immune system. They are mostly secreted by macrophages and lymphocytes and their production is induced in response to injury or infection. Their actions influence other cells of the immune system as well as other tissues and organs including the liver and brain. There are 18 ILs described up till now. IL-2, IL-4, IL-6, IL-8 and IL-10 are the important interleukins. The following table shows selected functions of representative interleukins.

TABLE 1
Selected Functions of Representative Interleukins*

| Functions | IL-1 | IL-2 | IL-4 | IL-6 | IL-8 | IL-10 |
|---|---|---|---|---|---|---|
| Enhance immune responses | + | + | + | + | − | + |
| Suppress immune responses | − | − | − | − | − | + |
| Enhance inflammation | + | + | + | + | + | − |
| Suppress inflammation | − | − | − | − | − | + |
| Promote cell growth | + | + | − | − | − | − |
| Chemotactic (chemokines) | − | − | − | − | + | − |
| Pyrogenic | + | − | − | − | − | − |

Interleukin-1β (IL-1β) is a 17 kDa secreted proinflammatory cytokine that is involved in the acute phase response and is a pathogenic mediator of many diseases. IL-1β is normally produced by macrophages and epithelial cells. IL-1β is also released from cells undergoing apoptosis. The normal serum concentration of IL-1β is <30 pg/ml (1.8 pM). In theory, IL-1β would be elevated earlier than other acute phase proteins such as CRP in unstable angina and acute myocardial infarction, since IL-1β is an early participant in the acute phase response. Furthermore, IL-1β is released from cells undergoing apoptosis, which may be activated in the early stages of ischemia. In this regard, elevation of the plasma IL-1β concentration associated with ACS requires further investigation using a high-sensitivity assay. Elevations of the plasma IL-1β concentration are associated with activation of the acute phase response in proinflammatory conditions such as trauma and infection. IL-1β has a biphasic physiological half-life of 5 minutes followed by 4 hours (Kudo, S. et al., Cancer Res. 50:5751-5755, 1990). IL-1β is released into the extracellular milieu upon activation of the inflammatory response or apoptosis.

Interleukin-1 receptor antagonist (IL-1ra) is a 17 kDa member of the IL-1 family predominantly expressed in hepatocytes, epithelial cells, monocytes, macrophages, and neutrophils. IL-1ra has both intracellular and extracellular forms produced through alternative splicing. IL-1ra is thought to participate in the regulation of physiological IL-1 activity. IL-1ra has no IL-1-like physiological activity, but is able to bind the IL-1 receptor on T-cells and fibroblasts with an affinity similar to that of IL-1β, blocking the binding of IL-1α and IL-1β and inhibiting their bioactivity (Stockman, B. J. et al., Biochemistry 31:5237-5245, 1992; Eisenberg, S. P. et al., Proc. Natl. Acad. Sci. U.S.A. 88:5232-5236, 1991; Carter, D. B. et al., Nature 344:633-638, 1990). IL-1ra is normally present in higher concentrations than IL-1 in plasma, and it has been suggested that IL-1ra levels are a better correlate of disease severity than IL-1 (Biasucci, L. M. et al., Circulation 99:2079-2084, 1999). Furthermore, there is evidence that IL-1ra is an acute phase protein (Gabay, C. et al., J. Clin. Invest. 99:2930-2940, 1997). The normal plasma concentration of IL-Ira is <200 pg/ml (12 pM). The plasma concentration of IL-1ra is elevated in patients with acute myocardial infarction and unstable angina that proceeded to acute myocardial infarction, death, or refractory angina (Biasucci, L. M. et al., Circulation 99:2079-2084, 1999; Latini, R. et al., J. Cardiovasc. Pharmacol. 23:1-6, 1994). Furthermore, IL-1ra was significantly elevated in severe acute myocardial infarction as compared to uncomplicated acute myocardial infarction (Latini, R. et al., J. Cardiovasc. Pharmacol. 23:1-6, 1994). Elevations in the plasma concentration of IL-1ra are associated with any condition that involves activation of the inflammatory or acute phase response, including infection, trauma, and arthritis. IL-1ra is released into the bloodstream in pro-inflammatory conditions, and it may also be released as a participant in the acute phase response. The major sources of clearance of IL-1ra from the bloodstream appear to be kidney and liver (Kim, D. C. et al., J. Pharm. Sci. 84:575-580, 1995). IL-1ra concentrations were elevated in the plasma of individuals with unstable angina within 24 hours of onset, and these elevations may even be evident within 2 hours of onset (Biasucci, L. M. et al., Circulation 99:2079-2084, 1999). In patients with severe progression of unstable angina, the plasma concentration of IL-1ra was higher 48 hours after onset than levels at admission, while the concentration decreased in patients with uneventful progression (Biasucci, L. M. et al., Circulation 99:2079-2084, 1999). In addition, the plasma concentration of IL-1ra associated with unstable angina can approach 1.4 ng/ml (80 pM). Changes in the plasma concentration of IL-1ra appear to be related to disease severity. Furthermore, it is likely released in conjunction with or soon after IL-1 release in pro-inflammatory conditions, and it is found at higher concentrations than IL-1. This indicates that IL-1ra may be a useful indirect marker of IL-1 activity, which elicits the production of IL-6.

Interleukin-6 (IL-6) is a 20 kDa secreted protein that is a hematopoietin family proinflammatory cytokine. IL-6 is an acute-phase reactant and stimulates the synthesis of a variety of proteins, including adhesion molecules. Its major function is to mediate the acute phase production of hepatic proteins, and its synthesis is induced by the cytokine IL-1. IL-6 is normally produced by macrophages and T lymphocytes. The normal serum concentration of IL-6 is <3 pg/ml (0.15 pM). The plasma concentration of IL-6 is elevated in patients with acute myocardial infarction and unstable angina, to a greater degree in acute myocardial infarction (Biasucci, L. M. et al., Circulation 94:874-877, 1996; Manten, A. et al., Cardiovasc. Res. 40:389-395, 1998; Biasucci, L. M. et al., Circulation 99:2079-2084, 1999). IL-6 is not significantly elevated in the plasma of patients with stable angina (Biasucci, L. M. et al., Circulation 94:874-877, 1996; Manten, A. et al., Cardiovasc. Res. 40:389-395, 1998). Furthermore, IL-6 concentrations increase over 48 hours from onset in the plasma of patients with unstable angina with severe progression, but decrease in those with uneventful progression (Biasucci, L. M. et al., Circulation 99:2079-2084, 1999). This indicates that IL-6 may be a useful indicator of disease progression. Plasma elevations of IL-6 are associated with any nonspecific proinflammatory condition such as trauma, infection, or other diseases that elicit an acute phase response. IL-6 has a half-life of 4.2 hours in the bloodstream and is elevated following acute myocardial infarction and unstable angina (Manten, A. et al., Cardiovasc. Res. 40:389-395, 1998). The plasma concentration of IL-6 is elevated within 8-12 hours of acute myocardial infarction onset, and can approach 100 pg/ml. The plasma concentration of IL-6 in patients with unstable angina was elevated at peak levels 72 hours after onset, possibly due to the severity of insult (Biasucci, L. M. et al., Circulation 94:874-877, 1996).

Interleukin-8 (IL-8) is a 6.5 kDa chemokine produced by monocytes, endothelial cells, alveolar macrophages and fibroblasts. IL-8 induces chemotaxis and activation of neutrophils and T cells.

Tumor necrosis factor α (TNFβ) is a 17 kDa secreted proinflammatory cytokine that is involved in the acute phase response and is a pathogenic mediator of many diseases. TNFα is normally produced by macrophages and natural killer cells. TNF-alpha is a protein of 185 amino acids glycosylated at positions 73 and 172. It is synthesized as a precursor protein of 212 amino acids. Monocytes express at least five different molecular forms of TNF-alpha with molecular masses of 21.5-28 kDa. They mainly differ by post-translational alterations such as glycosylation and phosphorylation. The normal serum concentration of TNFα is <40 pg/ml (2 pM). The plasma concentration of TNFα is elevated in patients with acute myocardial infarction, and is marginally elevated in patients with unstable angina (Li, D. et al., Am. Heart J 137:1145-1152, 1999; Squadrito, F. et al., Inflamm. Res. 45:14-19, 1996; Latini, R. et al., J. Cardiovasc. Pharmacol. 23:1-6, 1994; Carlstedt, F. et al., J. Intern. Med. 242:361-365, 1997). Elevations in the plasma concentration of TNFα are associated with any proinflammatory condition, including trauma, stroke, and infection. TNFα has a half-life of approximately 1 hour in the bloodstream, indicating that it may be removed from the circulation soon after symptom onset. In patients with acute myocardial infarction, TNFα was elevated 4 hours after the onset of chest pain, and gradually declined to normal levels within 48 hours of onset (Li, D. et al., Am. Heart J. 137:1145-1152, 1999). The concentration of TNFα in the plasma of acute myocardial infarction patients exceeded 300 pg/ml (15 pM) (Squadrito, F. et al., Inflamm. Res. 45:14-19, 1996). Release of TNFα by monocytes has also been related to the progression of pneumoconiosis in caol workers. Schins and Borm, Occup. Environ. Med. 52: 441-50 (1995).

Soluble intercellular adhesion molecule (sICAM-1), also called CD54, is a 85-110 kDa cell surface-bound immunoglobulin-like integrin ligand that facilitates binding of leukocytes to antigen-presenting cells and endothelial cells during leukocyte recruitment and migration. sICAM-1 is normally produced by vascular endothelium, hematopoietic stem cells and non-hematopoietic stem cells, which can be found in intestine and epidermis. sICAM-1 can be released from the cell surface during cell death or as a result of proteolytic activity. The normal plasma concentration of sICAM-1 is approximately 250 ng/ml (2.9 nM). The plasma concentration of sICAM-1 is significantly elevated in patients with acute myocardial infarction and unstable angina, but not stable angina (Pellegatta, F. et al., J. Cardiovasc. Pharmacol. 30:455-460, 1997; Miwa, K. et al., Cardiovasc. Res. 36:37-44, 1997; Ghaisas, N. K. et al., Am. J. Cardiol. 80:617-619, 1997; Ogawa, H. et al., Am. J. Cardiol. 83:38-42, 1999). Furthermore, ICAM-1 is expressed in atherosclerotic lesions and in areas predisposed to lesion formation, so it may be released into the bloodstream upon plaque rupture (Iiyama, K. et al., Circ. Res. 85:199-207, 1999; Tenaglia, A. N. et al., Am. J. Cardiol. 79:742-747, 1997). Elevations of the plasma concentration of sICAM-1 are associated with ischemic stroke, head trauma, atherosclerosis, cancer, preeclampsia, multiple sclerosis, cystic fibrosis, and other nonspecific inflammatory states (Kim, J. S., J. Neurol. Sci. 137:69-78, 1996; Laskowitz, D. T. et al., J. Stroke Cerebrovasc. Dis. 7:234-241, 1998). The plasma concentration of sICAM-1 is elevated during the acute stage of acute myocardial infarction and unstable angina. The elevation of plasma sICAM-1 reaches its peak within 9-12 hours of acute myocardial infarction onset, and returns to normal levels within 24 hours (Pellegatta, F. et al., J. Cardiovasc. Pharmacol. 30:455-460, 1997). The plasma concentration of sICAM can approach 700 ng/ml (8 nM) in patients with acute myocardial infarction (Pellegatta, F. et al., J. Cardiovasc. Pharmacol. 30:455-460, 1997). sICAM-1 is elevated in the plasma of individuals with acute myocardial infarction and unstable angina, but it is not specific for these diseases. It may, however, be useful marker in the differentiation of acute myocardial infarction and unstable angina from stable angina since plasma elevations are not associated with stable angina. Interestingly, ICAM-1 is present in atherosclerotic plaques, and may be released into the bloodstream upon plaque rupture.

Vascular cell adhesion molecule (VCAM), also called CD106, is a 100-110 kDa cell surface-bound immunoglobulin-like integrin ligand that facilitates binding of B lymphocytes and developing T lymphocytes to antigen-presenting cells during lymphocyte recruitment. VCAM is normally produced by endothelial cells, which line blood and lymph vessels, the heart, and other body cavities. VCAM-1 can be released from the cell surface during cell death or as a result of proteolytic activity. The normal serum concentration of sVCAM is approximately 650 ng/ml (6.5 nM). The plasma concentration of sVCAM-1 is marginally elevated in patients with acute myocardial infarction, unstable angina, and stable angina (Mulvihill, N. et al., Am. J. Cardiol. 83:1265-7, A9, 1999; Ghaisas, N. K. et al., Am. J. Cardiol. 80:617-619, 1997). However, sVCAM-1 is expressed in atherosclerotic lesions and its plasma concentration may correlate with the extent of atherosclerosis (Iiyama, K. et al., Circ. Res. 85:199-207, 1999; Peter, K. et al., Arterioscler. Thromb. Vasc. Biol. 17:505-512, 1997). Elevations in the plasma concentration of sVCAM-1 are associated with ischemic stroke, cancer, diabetes, preeclampsia, vascular injury, and other nonspecific inflammatory states (Bitsch, A. et al., Stroke 29:2129-2135, 1998; Otsuki, M. et al., Diabetes 46:2096-2101, 1997; Banks, R. E. et al., Br. J. Cancer 68:122-124, 1993; Steiner, M. et al., Thromb. Haemost. 72:979-984, 1994; Austgulen, R. et al., Eur. J. Obstet. Gynecol. Reprod. Biol. 71:53-58, 1997).

Monocyte chemotactic protein-1 (MCP-1) is a 10 kDa chemotactic factor that attracts monocytes and basophils, but not neutrophils or eosiniphils. MCP-1 is normally found in equilibrium between a monomeric and homodimeric form, and it is normally produced in and secreted by monocytes and vascular endothelial cells (Yoshimura, T. et al., FEBS Lett. 244:487-493, 1989; Li, Y. S. et al., Mol. Cell. Biochem. 126:61-68, 1993). MCP-1 has been implicated in the pathogenesis of a variety of diseases that involve monocyte infiltration, including psoriasis, rheumatoid arthritis, and atherosclerosis. The normal concentration of MCP-1 in plasma is <0.1 ng/ml. The plasma concentration of MCP-1 is elevated in patients with acute myocardial infarction, and may be elevated in the plasma of patients with unstable angina, but no elevations are associated with stable angina (Soejima, H. et al., J. Am. Coll. Cardiol. 34:983-988, 1999; Nishiyama, K. et al., Jpn. Circ. J 62:710-712, 1998; Matsumori, A. et al., J. Mol. Cell. Cardiol. 29:419-423, 1997). Interestingly, MCP-1 also may be involved in the recruitment of monocytes into the arterial wall during atherosclerosis. Elevations of the serum concentration of MCP-1 are associated with various conditions associated with inflammation, including alcoholic liver disease, interstitial lung disease, sepsis, and systemic lupus erythematosus (Fisher, N. C. et al., Gut 45:416-420, 1999; Suga, M. et al., Eur. Respir. J. 14:376-382, 1999; Bossink, A. W. et al., Blood 86:3841-3847, 1995; Kaneko, H. et al. J. Rheumatol. 26:568-573, 1999). MCP-1 is released into the bloodstream upon activation of monocytes and endothelial cells. The concentration of MCP-1 in plasma form patients with acute myocardial infarction has been reported to approach 1 ng/ml (100 pM), and can remain elevated for one month (Soejima, H. et al., J. Am. Coll. Cardiol. 34:983-988, 1999). MCP-1 is a specific marker of the presence of a pro-inflammatory condition that involves monocyte migration.

Hemoglobin (Hb) is an oxygen-carrying iron-containing globular protein found in erythrocytes. It is a heterodimer of two globin subunits. α₂γ₂ is referred to as fetal Hb, α₂β₂ is called adult HbA, and α₂δ₂ is called adult HbA₂. 90-95% of hemoglobin is HbA, and the α₂ globin chain is found in all Hb types, even sickle cell hemoglobin. Hb is responsible for carrying oxygen to cells throughout the body. Hbα₂ is not normally detected in serum.

Human lipocalin-type prostaglandin D synthase (hPDGS), also called β-trace, is a 30 kDa glycoprotein that catalyzes the formation of prostaglandin D2 from prostaglandin H. The upper limit of hPDGS concentrations in apparently healthy individuals is reported to be approximately 420 ng/ml (Patent No. EP0999447A1). Elevations of hPDGS have been identified in blood from patients with unstable angina and cerebral infarction (Patent No. EP0999447A1). Furthermore, hPDGS appears to be a useful marker of ischemic episodes, and concentrations of hPDGS were found to decrease over time in a patient with angina pectoris following percutaneous transluminal coronary angioplasty (PTCA), suggesting that the hPGDS concentration decreases as ischemia is resolved (Patent No. EP0999447A1).

Mast cell tryptase, also known as alpha tryptase, is a 275 amino acid (30.7 kDa) protein that is the major neutral protease present in mast cells. Mast cell tryptase is a specific marker for mast cell activation, and is a marker of allergic airway inflammation in asthma and in allergic reactions to a diverse set of allergens. See, e.g., Taira et al., J. Asthma 39: 315-22 (2002); Schwartz et al., N. Engl. J. Med. 316: 1622-26 (1987). Elevated serum tryptase levels (>1 ng/mL) between 1 and 6 hours after an event provides a specific indication of mast cell degranulation.

Eosinophil cationic protein (ECP) is a heterogeneous protein with molecular weight variants from 16-24 kDa and a pI of pH 10.8. ECP is highly cytotoxic and is released by activated eosinophils. Venge, Clinical and experimental allergy, 23 (suppl. 2): 3-7 (1993). Concentrations of ECP in the bronchoalveolar lavage fluid (BALF) of asthma patients vary with the severity of their disease, and ECP concentrations in sputum have also been shown to reflect the pathophysiology of the disease. Bousquet et al., New Engl. J. Med. 323: 1033-9 (1990). Virchow et al., Am. Rev. Respir. Dis. 146: 604-6 (1992). Assessment of serum ECP may be assumed to reflect pulmonary inflammation in bronchial asthma. Koller et al., Arch. Dis. Childhood 73: 413-7 (1995); see also, Sorkness et al., Clin. Exp. Allergy 32: 1355-59 (2002); Badr-elDin et al., East Mediterr. Health J. 5: 664-75 (1999).

KL-6 (also referred to as MUC1) is a high molecular weight (>300 kDa) mucinous glycoprotein expressed on pneumonocytes. Serum levels of KL-6 are reportedly elevated in interstitial lung diseases, which are characterized by exertional dyspnea. KL-6 has been shown to be a marker of various interstitial lung diseases, including pulmonary fibrosis, interstitial pneumonia, sarcoidosis, and interstitial pneumonitis. See, e.g., Kobayashi and Kitamura, Chest 108: 311-15 (1995); Kohno, J. Med. Invest. 46: 151-58 (1999); Bandoh et al., Ann. Rheum. Dis. 59: 257-62 (2000); and Yamane et al., J. Rheumatol. 27: 930-4 (2000).

Procalcitonin is a 116 amino acid (14.5 kDa) protein encoded by the Calc-1 gene located on chromosome 11p15.4. The Calc-1 gene produces two transcripts that are the result of alternative splicing events. Pre-procalcitonin contains a 25 amino acid signal peptide which is processed by C-cells in the thyrois to a 57 amino acid N-terminal fragment, a 32 amino acid calcitonin fragment, and a 21 amino acid katacalcin fragment. Procalcitonin is secreted intact as a glycosylated product by other body cells. Whicher et al., Ann. Clin. Biochem. 38: 483-93 (2001). Plasma procalcitonin has been identified as a marker of sepsis and its severity (Yukioka et al., Ann. Acad. Med. Singapore 30: 528-31 (2001)), with day 2 procalcitonin levels predictive of mortality (Pettila et al., Intensive Care Med. 28: 1220-25 (2002).

Interleukin 10 (“IL-0”) is a 160 amino acid (18.5 kDa predicted mass) cytokine that is a member of the four α-helix bundle family of cytokines. In solution, IL-10 forms a homodimer having an apparent molecular weight of 39 kDa. The human IL-10 gene is located on chromosome 1. Viera et al., Proc. Natl. Acad. Sci. USA 88: 1172-76 (1991); Kim et al., J. Immunol. 148: 3618-23 (1992). Overproduction of IL-10 has been identified as a marker in sepsis, and is predictive of severity and mortality. Gogos et al., J. Infect. Dis. 181: 176-80 (2000).

(v) Exemplary Specific Markers for Neural Tissue Injury

Adenylate kinase (AK) is a ubiquitous 22 kDa cytosolic enzyme that catalyzes the interconversion of ATP and AMP to ADP. Four isoforms of adenylate kinase have been identified in mammalian tissues (Yoneda, T. et al., Brain Res Mol Brain Res 62:187-195, 1998). The AK1 isoform is found in brain, skeletal muscle, heart, and aorta. The normal serum mass concentration of AK1 is currently unknown, because a functional assay is typically used to measure total AK concentration. The normal serum AK concentration is <5 units/liter and AK elevations have been performed using CSF (Bollensen, E. et al., Acta Neurol Scand 79:53-582, 1989). Serum AK1 appears to have the greatest specificity of the AK isoforms as a marker of cerebral injury. AK may be best suited as a cerebrospinal fluid marker of cerebral ischemia, where its dominant source would be neural tissue.

Neurotrophins are a family of growth factors expressed in the mammalian nervous system. Some examples include nerve growth factor (NGF), brain-derived neurotrophic factor (BDNF), neurotrophin-3 (NT-3) and neurotrophin-4/5 (NT-4/5). Neurotrophins exert their effects primarily as target-derived paracrine or autocrine neurotrophic factors. The role of the neurotrophins in survival, differentiation and maintenance of neurons is well known. They exhibit partially overlapping but distinct patterns of expression and cellular targets. In addition to the effects in the central nervous system, neurotrophins also affect peripheral afferent and efferent neurons.

BDNF is a potent neurotrophic factor which supports the growth and survivability of nerve and/or glial cells. BDNF is expressed as a 32 kDa precursor “pro-BDNF” molecule that is cleaved to a mature BDNF form. Mowla et al., J. Biol. Chem. 276: 12660-6 (2001). The most abundant active form of human BDNF is a 27 kDa homodimer, formed by two identical 119 amino acid subunits, which is held together by strong hydrophobic interactions; however, pro-BDNF is also released extracellularly and is biologically active. BDNF is widely distributed throughout the CNS and displays in vitro trophic effects on a wide range of neuronal cells, including hippocampal, cerebellar, and cortical neurons. In vivo, BDNF has been found to rescue neural cells from traumatic and toxic brain injury. For example, studies have shown that after transient middle cerebral artery occlusion, BDNF mRNA is upregulated in cortical neurons (Schabiltz et al., J. Cereb. Blood Flow Metab. 14:500-506, 1997). In experimentally induced focal, unilateral thrombotic stroke, BDNF mRNA was increased from 2 to 18 h following the stroke. Such results suggest that BDNF potentially plays a neuroprotective role in focal cerebral ischemia.

NT-3 is also a 27 kDa homodimer consisting of two 119-amino acid subunits. The addition of NT-3 to primary cortical cell cultures has been shown to exacerbate neuronal death caused by oxygen-glucose deprivation, possible via oxygen free radical mechanisms (Bates et al., Neurobiol. Dis. 9:24-37, 2002). NT-3 is expressed as an inactive pro-NT-3 molecule, which is cleaved to the mature biologically active form.

Calbindin-D is a 28 kDa cytosolic vitamin D-dependent Ca²⁺-binding protein that may serve a cellular protective function by stabilizing intracellular calcium levels. Calbindin-D is found in the central nervous system, mainly in glial cells, and in cells of the distal renal tubule (Hasegawa, S. et al., J. Urol. 149:1414-1418, 1993). The normal serum concentration of calbindin-D is <20 pg/ml (0.7 pM). Serum calbindin-D concentration is reportedly elevated following cardiac arrest, and this elevation is thought to be a result of CNS damage due to cerebral ischemia (Usui, A. et al., J. Neurol. Sci. 123:134-139, 1994). Elevations of serum calbindin-D are elevated and plateau soon after reperfusion following ischemia. Maximum serum calbindin-D concentrations can be as much as 700 pg/ml (25 pM).

Creatine kinase (CK) is a cytosolic enzyme that catalyzes the reversible formation of ADP and phosphocreatine from ATP and creatine. The brain-specific CK isoform (CK-BB) is an 85 kDa cytosolic protein that accounts for approximately 95% of the total brain CK activity. It is also present in significant quantities in cardiac tissue, intestine, prostate, rectum, stomach, smooth muscle, thyroid uterus, urinary bladder, and veins (Johnsson, P. J., Cardiothorac. Vasc. Anesth. 10: 120-126, 1996). The normal serum concentration of CK-BB is <10 ng/ml (120 pM). Serum CK-BB is elevated after hypoxic and ischemic brain injury, but a further investigation is needed to identify serum elevations in specific stroke types (Laskowitz, D. T. et al., J. Stroke Cerebrovasc. Dis. 7:234-241, 1998). Elevations of CK-BB in serum can be attributed to cerebral injury due to ischemia, coupled with increased permeability of the blood brain barrier. No correlation of the serum concentration of CK-BB with the extent of damage (infarct volume) or neurological outcome has been established. CK-BB has a half-life of 1-5 hours in serum and is normally detected in serum at a concentration of <10 ng/ml (120 pM). In severe stroke, serum concentrations CK-BB are elevated and peak soon after the onset of stroke (within 24 hours), gradually returning to normal after 3-7 days (4). CK-BB concentrations in the serum of individuals with head injury peak soon after injury and return to normal between 3.5-12 hours after injury, depending on the injury severity (Skogseid, I. M. et al., Acta Neurochir. (Wien.) 115:106-111, 1992). Maximum serum CK-BB concentrations can exceed 250 ng/ml (3 rM). CK-BB may be best suited as a CSF marker of cerebral ischemia, where its dominant source would be neural tissue. CKBB might be more suitable as a serum marker of CNS damage after head injury because it is elevated for a short time in these individuals, with its removal apparently dependent upon the severity of damage.

Glial fibrillary acidic protein (GFAP) is a 55 kDa cytosolic protein that is a major structural component of astroglial filaments and is the major intermediate filament protein in astrocytes. GFAP is specific to astrocytes, which are interstitial cells located in the CNS and can be found near the blood-brain barrier. GFAP is not normally detected in serum. Serum GFAP is elevated following ischemic stroke (Niebroj-Dobosz, I., et al., Folia Neuropathol. 32:129-137, 1994). Current reports investigating serum GFAP elevations associated with stroke are severely limited, and much further investigation is needed to establish GFAP as a serum marker for all stroke types. Most studies investigating GFAP as a stroke marker have been performed using cerebrospinal fluid. Elevations of GFAP in serum can be attributed to cerebral injury due to ischemia, coupled with increased permeability of the blood brain barrier. No correlation of the serum concentration of GFAP with the extent of damage (infarct volume) or neurological outcome has been established. GFAP is elevated in cerebrospinal fluid of individuals with various neuropathies affecting the CNS, but there are no reports currently available describing the release of GFAP into the serum of individuals with diseases other than stroke (Albrechtsen, M. and Bock, E. J., Neuroimmunol. 8:301-309, 1985). Serum concentrations GFAP appear to be elevated soon after the onset of stroke, continuously increase and persist for an amount of time (weeks) that may correlate with the severity of damage. GFAP appears to a very specific marker for severe CNS injury, specifically, injury to astrocytes due to cell death caused by ischemia or physical damage.

Lactate dehydrogenase (LDH) is a ubiquitous 135 kDa cytosolic enzyme. It is a tetramer of A and B chains that catalyzes the reduction of pyruvate by NADH to lactate. Five isoforms of LDH have been identified in mammalian tissues, and the tissue-specific isoforms are made of different combinations of A and B chains. The normal serum mass concentration of LDH is currently unknown, because a functional assay is typically used to measure total LDH concentration. The normal serum LDH concentration is <600 units/liter (Ray, P. et al., Cancer Detect. Prev. 22:293-304, 1998). A great majority of investigations into LDH elevations in the context of stroke have been performed using cerebrospinal fluid, and elevations correlate with the severity of injury. Elevations in serum LDH activity are reported following both ischemic and hemorrhagic stroke, but further studies are needed in serum to confirm this observation and to determine a correlation with the severity of injury and neurological outcome (Aggarwal, S. P. et al., J. Indian Med. Assoc. 93:331-332, 1995; Maiuri, F. et al., Neurol. Res. 11:6-8, 1989). LDH may be best suited as a cerebrospinal fluid marker of cerebral ischemia, where its dominant source would be neural tissue.

Myelin basic protein (MBP) is actually a 14-21 kDa family of cytosolic proteins generated by alternative splicing of a single MBP gene that is likely involved in myelin compaction around axons during the myelination process. MBP is specific to oligodendrocytes in the CNS and in Schwann cells of the peripheral nervous system (PNS). It accounts for approximately 30% of the total myelin protein in the CNS and approximately 10% of the total myelin protein in the PNS. The normal serum concentration of MBP is <7 ng/ml (400 pM). Serum MBP is elevated after all types of severe stroke, specifically thrombotic stroke, embolic stroke, intracerebral hemorrhage, and subarachnoid hemorrhage, while elevations in MBP concentration are not reported in the serum of individuals with strokes of minor to moderate severity, which would include lacunar infarcts or transient ischemic attacks (Palfreyman, J. W. et al., Clin. Chim. Acta 92:403-409, 1979). Elevations of MBP in serum can be attributed to cerebral injury due to physical damage or ischemia caused by infarction or cerebral hemorrhage, coupled with increased permeability of the blood brain barrier. The serum concentration of MBP has been reported to correlate with the extent of damage (infarct volume), and it may also correlate with neurological outcome. The amount of available information regarding serum MBP elevations associated with stroke is limited, because most investigations have been performed using cerebrospinal fluid. MBP is normally detected in serum at an upper limit of 7 ng/ml (400 pM), is elevated after severe stroke and cerebral injury. Serum MBP is thought to be elevated within hours after stroke onset, with concentrations increasing to a maximum level within 2-5 days after onset. After the serum concentration reaches its maximum, which can exceed 120 ng/ml (6.9 nM), it can take over one week to gradually decrease to normal concentrations. Because the severity of damage has a direct effect on the release of MBP, it will affect the release kinetics by influencing the length of time that MBP is elevated in the serum. MBP will be present in the serum for a longer period of time as the severity of injury increases. The release of MBP into the serum of patients with head injury is thought to follow similar kinetics as those described for stroke, except that serum MBP concentrations reportedly correlate with the neurological outcome of individuals with head injury (Thomas, D. G. et al., Acta Neurochir. Suppl. (Wien) 28:93-95, 1979). The release of MBP into the serum of patients with intracranial tumors is thought to be persistent, but still needs investigation. Finally, serum MBP concentrations can sometimes be elevated in individuals with demyelinating diseases, but no conclusive investigations have been reported. As reported in individuals with multiple sclerosis, MBP is frequently elevated in the cerebrospinal fluid, but matched elevations in serum are often not present (Jacque, C. et al., Arch. Neurol. 39:557-560, 1982). This could indicate that cerebral damage has to be accompanied by an increase in the permeability of the blood-brain barrier to result in elevation of serum MBP concentrations. However, MBP can also be elevated in the population of individuals having intracranial tumors. The presence of these individuals in the larger population of individuals that would be candidates for an assay using this marker for stroke is rare. These individuals, in combination with individuals undergoing neurosurgical procedures or with demyelinating diseases, would nonetheless have an impact on determining the specificity of MBP for cerebral injury. Additionally, serum MBP may be useful as a marker of severe stroke, potentially identifying individuals that would not benefit from stroke therapies and treatments, such as tPA administration.

Neural cell adhesion molecule (NCAM), also called CD56, is a 170 kDa cell surface-bound immunoglobulin-like integrin ligand that is involved in the maintenance of neuronal and glial cell interactions in the nervous system, where it is expressed on the surface of astrocytes, oligodendrocytes, Schwann cells, neurons, and axons. NCAM is also localized to developing skeletal muscle myotubes, and its expression is upregulated in skeletal muscle during development, denervation and renervation. The normal serum mass concentration of NCAM has not been reported. NCAM is commonly measured by a functional enzyme immunoassay and is reported to have a normal serum concentration of <20 units/ml. Changes in serum NCAM concentrations specifically related to stroke have not been reported. NCAM may be best suited as a CSF marker of cerebral ischemia, where its dominant source would be neural tissue.

Enolase is a 78 kDa homo- or heterodimeric cytosolic protein produced from α, β, and γ subunits. It catalyzes the interconversion of 2-phosphoglycerate and phosphoenolpyruvate in the glycolytic pathway. Enolase can be present as αα, ββ, αγ, and γγ isoforms. The α subunit is found in glial cells and most other tissues, the β subunit is found in muscle tissue, and the γ subunit if found mainly in neuron al and neuroendocrine cells (Quinn, G. B. et al., Clin. Chem. 40:790-795, 1994). The γγ enolase isoform is most specific for neurons, and is referred to as neuron-specific enolase (NSE). NSE, found predominantly in neurons and neuroendocrine cells, is also present in platelets and erythrocytes. The normal serum concentration of NSE is <12.5 ng/ml (160 pM). NSE is made up of two subunits; thus, the most feasible immunological assay used to detect NSE concentrations would be one that is directed against one of the subunits. In this case, the γ subunit would be the ideal choice. However, the γ subunit alone is not as specific for cerebral tissue as the γ isoform, since a measurement of the γ subunit alone would detect both the αγ and γγ isoforms. In this regard, the best immunoassay for NSE would be a two-site assay that could specifically detect the γγ isoform. Serum NSE is reportedly elevated after all stroke types, including TIAs, which are cerebral in origin and are thought to predispose an individual to having a more severe stroke at a later date (Isgro, F. et al., Eur. J. Cardiothorac. Surg. 11:640-644, 1997). Elevations of NSE in serum can be attributed to cerebral injury due to physical damage or ischemia caused by infarction or cerebral hemorrhage, coupled with increased permeability of the blood brain barrier, and the serum concentration of NSE has been reported to correlate with the extent of damage (infarct volume) and neurological outcome (Martens, P. et al., Stroke 29:2363-2366, 1998). Additionally, a secondary elevation of serum NSE concentration may be an indicator of delayed neuronal injury resulting from cerebral vasospasm (Laskowitz, D. T. et al., J. Stroke Cerebrovasc. Dis. 7, 234-241, 1998). NSE, which has a biological half-life of 48 hours and is normally detected in serum at an upper limit of 12.5 ng/ml (160 pM), is elevated after stroke and cerebral injury. Serum NSE is elevated after 4 hours from stroke onset, with concentrations reaching a maximum 1-3 days after onset (Missler, U. et al., Stroke 28:1956-1960, 1997). After the serum concentration reaches its maximum, which can exceed 300 ng/ml (3.9 nM), it gradually decreases to normal concentrations over approximately one week. Because the severity of damage has a direct effect on the release of NSE, it will affect the release kinetics by influencing the length of time that NSE is elevated in the serum. NSE will be present in the serum for a longer period of time as the severity of injury increases. The release of NSE into the serum of patients with head injury follows different kinetics as seen with stroke, with the maximum serum concentration being reached within 1-6 hours after injury, often returning to baseline within 24 hours (Skogseid, I. M. et al., Acta Neurochir. (Wien.) 115:106-111, 1992). NSE is a specific marker for cerebral injury, specifically, injury to neuronal cells due to cell death caused by ischemia or physical damage. Neurons are about 10-fold less abundant in the brain than glial cells, so any cerebral injury coupled with increased permeability of the blood-brain barrier will have to occur in a region that has a significant regional population of neurons to significantly increase the serum NSE concentration. In addition, elevated serum concentrations of NSE can also indicate complications related to cerebral injury after AMI and cardiac surgery. Elevations in the serum concentration of NSE correlate with the severity of damage and the neurological outcome of the individual. NSE can be used as a marker of all stroke types, including TIAs. However, NSE cannot be used to differentiate ischemic and hemorrhagic stroke, and it is elevated in the population of individuals having tumors with neuroendocrine features.

Proteolipid protein (PLP) is a 30 kDa integral membrane protein that is a major structural component of CNS myelin. PLP is specific to oligodendrocytes in the CNS and accounts for approximately 50% of the total CNS myelin protein in the central sheath, although extremely low levels of PLP have been found (<1%) in peripheral nervous system (PNS) myelin. The normal serum concentration of PLP is <9 ng/ml (300 pM). Serum PLP is elevated after cerebral infarction, but not after transient ischemic attack (Trotter, J. L. et al., Ann. Neurol. 14:554-558, 1983). Current reports investigating serum PLP elevations associated with stroke are severely limited. Elevations of PLP in serum can be attributed to cerebral injury due to physical damage or ischemia caused by infarction or cerebral hemorrhage, coupled with increased permeability of the blood brain barrier. Correlation of the serum concentration of PLP with the extent of damage (infarct volume) or neurological outcome has not been established. No investigations examining the release kinetics of PLP into serum and its subsequent removal have been reported, but maximum concentrations approaching 60 ng/ml (2 nM) have been reported in encephalitis patients, which nearly doubles the concentrations found following stroke. PLP appears to a very specific marker for severe CNS injury, specifically, injury to oligodendrocytes. The available information relating PLP serum elevations and stroke is severely limited. PLP is also elevated in the serum of individuals with various neuropathies affecting the CNS. The undiagnosed presence of these individuals in the larger population of individuals that would be candidates for an assay using this marker for stroke is rare.

S-100 is a 21 kDa homo- or heterodimeric cytosolic Ca²⁺-binding protein produced from α and β subunits. It is thought to participate in the activation of cellular processes along the Ca2+-dependent signal transduction pathway (Bonfrer, J. M. et al., Br. J. Cancer 77:2210-2214, 1998). S-100ao (αα isoform) is found in striated muscles, heart and kidney, S-100a (αβ isoform) is found in glial cells, but not in Schwann cells, and S-100b (ββ isoform) is found in high concentrations in glial cells and Schwann cells, where it is a major cytosolic component. The β subunit is specific to the nervous system, predominantly the CNS, under normal physiological conditions and, in fact, accounts for approximately 96% of the total S-100 protein found in the brain (Jensen, R. et al., J. Neurochem. 45:700-705, 1985). In addition, S-100β can be found in tumors of neuroendocrine origin, such as gliomas, melanomas, Schwannomas, neurofibromas, and highly differentiated neuroblastomas, like ganglioneuroblastoma and ganglioneuroma (Persson, L. et al., Stroke 18:911-918, 1987). The normal serum concentration of S-100β is <0.2 ng/ml (19 pM), which is the detection limit of the immunological detection assays used. Serum S-100β is elevated after all stroke types, including TIAs. Elevations of S-100β in serum can be attributed to cerebral injury due to physical damage or ischemia caused by infarction or cerebral hemorrhage, coupled with increased permeability of the blood-brain barrier, and the serum concentration of S-100b has been shown to correlate with the extent of damage (infarct volume) and neurological outcome (Martens, P. et al., Stroke 29:2363-2366, 1998; Missler, U. et al., Stroke 28:1956-1960, 1997). S-100b has a biological half-life of 2 hours and is not normally detected in serum, but is elevated after stroke and cerebral injury. Serum S-100β is elevated after 4 hours from stroke onset, with concentrations reaching a maximum 2-3 days after onset. After the serum concentration reaches its maximum, which can approach 20 ng/ml (1.9 mM), it gradually decreases to normal over approximately one week. Because the severity of damage has a direct effect on the release of S-100b, it will affect the release kinetics by influencing the length of time that S-100b is elevated in the serum. S-100b will be present in the serum for a longer period of time as the seventy of injury increases. The release of S-100b into the serum of patients with head injury seems to follow somewhat similar kinetics as reported with stroke, with the only exception being that serum S-100β can be detected within 2.5 hours of onset and the maximum serum concentration is reached approximately 1 day after onset (Woertgen, C. et al., Acta Neurochir. (Wien) 139:1161-1164, 1997). S-100β is a specific marker for cerebral injury, specifically, injury to glial cells due to cell death caused by ischemia or physical damage. Glial cells are about 10 times more abundant in the brain than neurons, so any cerebral injury coupled with increased permeability of the blood-brain barrier will likely produce elevations of serum S-100β. Furthermore, elevated serum concentrations of S-100b can indicate complications related to cerebral injury after AMI and cardiac surgery. S-100b has been virtually undetectable in normal individuals, and elevations in its serum concentration correlate with the seventy of damage and the neurological outcome of the individual. S-100b can be used as a marker of all stroke types, including TIAs. However, S-100b cannot be used to differentiate ischemic and hemorrhagic stroke, and it is elevated in the population of individuals having neuroendocrine tumors, usually in advanced stages.

Thrombomodulin (TM) is a 70 kDa single chain integral membrane glycoprotein found on the surface of vascular endothelial cells. TM demonstrates anticoagulant activity by changing the substrate specificity of thrombin. The formation of a 1:1 stoichiometric complex between thrombin and TM changes thrombin function from procoagulant to anticoagulant. This change is facilitated by a change in thrombin substrate specificity that causes thrombin to activate protein C (an inactivator of factor Va and factor VIIIa), but not cleave fibrinogen or activate other coagulation factors (Davie, E. W. et al., Biochem. 30:10363-10370, 1991). The normal serum concentration of TM is 25-60 ng/ml (350-850 pM). Current reports describing serum TM concentration alterations following ischemic stroke are mixed, reporting no changes or significant increases (Seki, Y. et al., Blood Coagul. Fibrinolysis 8:391-396, 1997). Serum elevations of TM concentration reflect endothelial cell injury and would not indicate coagulation or fibrinolysis activation.

The gamma isoform of protein kinase C (PKCg) is specific for CNS tissue and is not normally found in the circulation. PKCg is activated during cerebral ischemia and is present in the ischemic penumbra at levels 2-24-fold higher than in contralateral tissue, but is not elevated in infarcted tissue (Krupinski, J. et al., Acta Neurobiol. Exp. (Warz) 58:13-21, 1998). In addition, animal models have identified increased levels of PKCg in the peripheral circulation of rats following middle cerebral artery occlusion (Cornell-Bell, A. et al., Patent No. WO 01/16599 A1). Additional isoforms of PKC, beta I and beta II were found in increased levels in the infarcted core of brain tissue from patients with cerebral ischemia (Krupinski, J. et al., Acta Neurobiol. Exp. (Warz) 58:13-21, 1998). Furthermore, the alpha and delta isoforms of PKC (PKCa and PKCd, respectively) have been implicated in the development of vasospasm following subarachnoid hemorrhage using a canine model of hemorrhage. PKCd expression was significantly elevated in the basilar artery during the early stages of vasospasm, and PKCa was significantly elevated as vasospasm progressed (Nishizawa, S. et al., Eur. J. Pharmacol. 398:113-119, 2000). Therefore, it may be of benefit to measure various isoforms of PKC, either individually or in various combinations thereof, for the identification of cerebral damage, the presence of the ischemic penumbra, as well as the development and progression of cerebral vasospasm following subarachnoid hemorrhage. Ratios of PKC isoforms such as PKCg and either PKCbI, PKCbII, or both also may be of benefit in identifying a progressing stroke, where the ischemic penumbra is converted to irreversibly damaged infarcted tissue. In this regard, PKCg may be used to identify the presence and volume of the ischemic penumbra, and either PKCbI, PKCbII, or both may be used to identify the presence and volume of the infarcted core of irreversibly damaged tissue during stroke. PKCd, PKCa, and ratios of PKCd and PKCa may be useful in identifying the presence and progression of cerebral vasospasm following subarachnoid hemorrhage.

(vi) Exemplary Non-Specific Markers Related to Coagulation and Hemostasis

Plasmin is a 78 kDa serine proteinase that proteolytically digests crosslinked fibrin, resulting in clot dissolution. The 70 kDa serine proteinase inhibitor α2-antiplasmin (α2AP) regulates plasmin activity by forming a covalent 1:1 stoichiometric complex with plasmin. The resulting ˜150 kDa plasmin-α2AP complex (PAP), also called plasmin inhibitory complex (PIC) is formed immediately after α2AP comes in contact with plasmin that is activated during fibrinolysis. The normal serum concentration of PAP is <1 μg/ml (6.9 nM). Serum PAP concentration is significantly elevated following embolic and hemorrhagic stroke, but not thrombotic or lacunar stroke, and the magnitude of elevation correlates with the severity of injury and neurological outcome (Seki, Y. et al., Am. J. Hematol. 50:155-160, 1995; Yamazaki, M. et al., Blood Coagul. Fibrinolysis 4:707-712, 1993; Uchiyama, S. et al., Semin. Thromb. Hemost. 23:535-541, 1997; Fujii, Y. et al., Neurosurgery 37:226-234, 1995). There are no reports that identify elevations in serum PAP concentration following cerebral transient ischemic attacks. Elevations in the serum concentration of PAP can be attributed to the activation of fibrinolysis. Elevations in the serum concentration of PAP may be associated with clot presence, or any condition that causes or is a result of fibrinolysis activation. These conditions can include atherosclerosis, disseminated intravascular coagulation, AMI, surgery, trauma, unstable angina, and thrombotic thrombocytopenic purpura. PAP is formed immediately following proteolytic activation of plasmin. Serum PAP is increased in embolic and hemorrhagic stroke. Serum concentrations are elevated soon after stroke onset and may persist for over 2 weeks (Fujii, Y. et al., J. Neurosurg. 86:594-602, 1997). In addition, serum PAP concentration may be higher in hemorrhagic stroke than in ischemic stroke. This could reflect the increased magnitude of coagulation activation associated with hemorrhage. Serum concentrations of PAP associated with stroke can approach 0.6 μg/ml (41 nM). PAP is a specific marker for fibrinolysis activation and the presence of a recent or continual hypercoagulable state. It is not specific for stroke or cerebral injury and can be elevated in many other disease states. However, it may be possible to use PAP to differentiate hemorrhagic stroke from ischemic stroke, which would be beneficial in ruling out patients for thrombolytic therapy, and to identify embolic vs. non-embolic ischemic strokes.

β-thromboglobulin (PTG) is a 36 kDa platelet a granule component that is released upon platelet activation. The normal serum concentration of βTG is <40 ng/ml (1.1 nM). Serum βTG concentration is elevated following ischemic and hemorrhagic stroke (Landi, G. et al., Neurol. 37:1667-1671, 1987). Serum elevations were not found to correlate with injury severity or neurological outcome. Investigations regarding βTG serum elevations in stroke are severely limited. Elevations in the serum PTG concentration can be attributed to platelet activation, which could indirectly indicate the presence of vascular injury. Elevations in the serum concentration of βTG may be associated with clot presence, or any condition that causes platelet activation. These conditions can include atherosclerosis, disseminated intravascular coagulation, AMI, surgery, trauma, unstable angina, and thrombotic thrombocytopenic purpura. βTG is released into the circulation immediately after platelet activation and aggregation. It has a biphasic half-life of 10 minutes, followed by an extended 1 hour half-life in serum (Switaiska, H. I. et al., J. Lab. Clin. Med. 106:690-700, 1985). Serum βTG concentration is reported to be elevated in various stroke types, but these studies may not be completely reliable. Special precautions must be taken to avoid platelet activation during the blood sampling process. Platelet activation is common during regular blood sampling, and could lead to artificial elevations of serum βTG concentration. In addition, the amount of βTG released into the bloodstream is dependent on the platelet count of the individual, which can be quite variable. Serum concentrations of PTG associated with stroke can approach 70 ng/ml (2 nM). βTG is a specific marker of platelet activation, but it is not specific for stroke or cerebral injury and can be elevated in many other disease states.

Platelet factor 4 (PF4) is a 40 kDa platelet a granule component that is released upon platelet activation. PF4 is a marker of platelet activation and has the ability to bind and neutralize heparin. The normal serum concentration of PF4 is <7 ng/ml (175 pM). Serum PF4 concentration is marginally elevated following intracerebral infarction, but not in individuals with intracerebral hemorrhage (Carter, A. M. et al., Arterioscler. Thromb. Vasc. Biol. 18:1124-1131, 1998). Additionally, serum PF4 concentrations are increased 5-9 days following subarachnoid hemorrhage, which may be related to the onset of cerebral vasospasm (Hirashima, Y. et al., Neurochem. Res. 22:1249-1255, 1997). Investigations regarding PF4 serum elevations in stroke are severely limited. Elevations in the serum PF4 concentration can be attributed to platelet activation, which could indirectly indicate the presence of vascular injury. Elevations in the serum concentration of PF4 may be associated with clot presence, or any condition that causes platelet activation. These conditions can include atherosclerosis, disseminated intravascular coagulation, AMI, surgery, trauma, unstable angina, and thrombotic thrombocytopenic purpura. PF4 is released into the circulation immediately after platelet activation and aggregation. It has a biphasic half-life of 1 minute, followed by an extended 20 minute half-life in serum. The half-life of PF4 in serum can be extended to 20-40 minutes by the presence of heparin (Rucinski, B. et al., Am. J. Physiol. 251:H800-H807, 1986). Special precautions must be taken to avoid platelet activation during the blood sampling process. Serum concentrations of PF4 associated with stroke can exceed 200 ng/ml (5 nM), but it is likely that this value may be influenced by platelet activation during the sampling procedure. Furthermore, the serum PF4 concentration would be dependent on platelet count, requiring a second variable to be determined along with the concentration estimates. Finally, patients taking aspirin or other platelet activation inhibitors would compromise the clinical usefulness of PF4 as a marker of platelet activation.

Fibrinopeptide A (FPA) is a 16 amino acid, 1.5 kDa peptide that is liberated from amino terminus of fibrinogen by the action of thrombin. Fibrinogen is synthesized and secreted by the liver. The normal serum concentration of FPA is <4 ng/ml (2.7 nM). Serum FPA is elevated after all stroke types, including cerebral transient ischemic attacks (TIAs) (Fon, E. A. et al., Stroke 25:282-286, 1994; Tohgi, H. et al., Stroke 21:1663-1667, 1990; Landi, G. et al., Neurol. 37:1667-1671, 1987). Elevations of FPA in serum can be attributed to coagulation activation, and the serum concentration of FPA has been reported to correlate with the neurological outcome, but not the severity or extent of damage (infarct volume) (Feinberg, W. M. et al., Stroke 27:1296-1300, 1996). Elevations in the serum concentration of FPA are associated with any condition that causes or is a result of coagulation activation. These conditions can include AMI, surgery, cancer, disseminated intravascular coagulation, nephrosis, thrombotic thrombocytopenic purpura, and unstable angina. FPA is released into the bloodstream immediately upon clot formation and it can remain elevated for more than 1 month. Maximum serum FPA concentrations following stroke can exceed 50 ng/ml (34 nM).

(vii) Other Non-Specific Markers for Cellular Injury

Human vascular endothelial growth factor (VEGF) is a dimeric protein, the reported activities of which include stimulation of endothelial cell growth, angiogenesis, and capillary permeability. VEGF is secreted by a variety of vascularized tissues. In an oxygen-deficient environment, vascular endothelial cells may be damaged and may not ultimately survive. However, such endothelial damage stimulates VEGF production by vascular smooth muscle cells. Vascular endothelial cells may exhibit increased survival in the presence of VEGF, an effect that is believed to be mediated by expression of Bcl-2. VEGF can exist as a variety of splice variants known as VEGF(189), VEGF(165), VEGF(164), VEGFB(155), VEGF(148), VEGF(145), and VEGF(121).

Insulin-like growth factor-1 (IGF-1) is a ubiquitous 7.5 kDa secreted protein that mediates the anabolic and somatogenic effects of growth hormone during development (1, 2). In the circulation, IGF-1 is normally bound to an IGF-binding protein that regulates IGF activity. The normal serum concentration of IGF-1 is approximately 160 ng/ml (21.3 nM). Serum IGF-1 concentrations are reported to be significantly decreased in individuals with ischemic stroke, and the magnitude of reduction appears to correlate with the severity of injury (Schwab, S. et al., Stroke 28:1744-1748, 1997). Decreased IGF-1 serum concentrations have been reported in individuals with trauma and massive activation of the immune system. Due to its ubiquitous expression, serum IGF-1 concentrations could also be decreased in cases of non-cerebral ischemia. Interestingly, IGF-1 serum concentrations are decreased following ischemic stroke, even though its cellular expression is upregulated in the infarct zone (Lee, W. H. and Bondy, C., Ann. N.Y. Acad. Sci. 679:418-422, 1993). The decrease in serum concentration could reflect an increased demand for growth factors or an increased metabolic clearance rate. Serum levels were significantly decreased 24 hours after stroke onset, and remained decreased for over 10 days (Schwab, S. et al., Stroke 28:1744-1748, 1997). Serum IGF-1 may be a sensitive indicator of cerebral injury. However, the ubiquitous expression pattern of IGF-1 indicates that all tissues can potentially affect serum concentrations of IGF-1, compromising the specificity of any assay using IGF-1 as a marker for stroke. In this regard, IGF-1 may be best suited as a cerebrospinal fluid marker of cerebral ischemia, where its dominant source would be neural tissue.

Adhesion molecules are involved in the inflammatory response can also be considered as acute phase reactants, as their expression levels are altered as a result of insult. Examples of such adhesion molecules include E-selectin, intercellular adhesion molecule-1, vascular cell adhesion molecule, and the like.

E-selectin, also called ELAM-1 and CD62E, is a 140 kDa cell surface C-type lectin expressed on endothelial cells in response to IL-1 and TNFα that mediates the “rolling” interaction of neutrophils with endothelial cells during neutrophil recruitment. The normal serum concentration of E-selectin is approximately 50 ng/ml (2.9 nM). Investigations into the changes on serum E-selectin concentrations following stroke have reported mixed results. Some investigations report increases in serum E-selectin concentration following ischemic stroke, while others find it unchanged (Bitsch, A. et al., Stroke 29:2129-2135, 1998; Kim, J. S., J. Neurol. Sci. 137:69-78, 1996; Shyu, K. G. et al., J. Neurol. 244:90-93, 1997). E-selectin concentrations are elevated in the CSF of individuals with subarachnoid hemorrhage and may predict vasospasm (Polin, R. S. et al., J. Neurosurg. 89:559-567, 1998). Elevations in the serum concentration of E-selectin would indicate immune system activation. Serum E-selectin concentrations are elevated in individuals with, atherosclerosis, various forms of cancer, preeclampsia, diabetes, cystic fibrosis, AMI, and other nonspecific inflammatory states (Hwang, S. J. et al., Circulation 96:4219-4225, 1997; Banks, R. E. et al., Br. J. Cancer 68:122-124, 1993; Austgulen, R. et al., Eur. J. Obstet. Gynecol. Reprod. Biol. 71:53-58, 1997; Steiner, M. et al., Thromb. Haemost. 72:979-984, 1994; De Rose, V. et al., Am. J. Respir. Crit. Care Med. 157:1234-1239, 1998). The serum concentration of E-selectin may be elevated following ischemic stroke, but it is not clear if these changes are transient or regulated by an as yet unidentified mechanism. Serum E-selectin may be a specific marker of endothelial cell injury. It is not, however, a specific marker for stroke or cerebral injury, since it is elevated in the serum of individuals with various conditions causing the generation of an inflammatory state. Furthermore, elevation of serum E-selectin concentration is associated with some of the risk factors associated with stroke.

Head activator (HA) is an 11 amino acid, 1.1 kDa neuropeptide that is found in the hypothalamus and intestine. It was originally found in the freshwater coelenterate hydra, where it acts as a head-specific growth and differentiation factor. In humans, it is thought to be a growth regulating agent during brain development. The normal serum HA concentration is <0.1 ng/ml (100 pM) Serum HA concentration is persistently elevated in individuals with tumors of neural or neuroendocrine origin (Schaller, H. C. et al., J Neurooncol. 6:251-258, 1988; Winnikes, M. et al., Eur. J. Cancer 28:421-424, 1992). No studies have been reported regarding HA serum elevations associated with stroke. HA is presumed to be continually secreted by tumors of neural or neuroendocrine origin, and serum concentration returns to normal following tumor removal. Serum HA concentration can exceed 6.8 ng/ml (6.8 nM) in individuals with neuroendocrine-derived tumors. The usefulness of HA as part of a stroke panel would be to identify individuals with tumors of neural or neuroendocrine origin. These individuals may have serum elevations of markers associated with cerebral injury as a result of cancer, not cerebral injury related to stroke. Although these individuals may be a small subset of the group of individuals that would benefit from a rapid diagnostic of cerebral injury, the use of HA as a marker would aid in their identification. Finally, angiotensin converting enzyme, a serum enzyme, has the ability to degrade HA, and blood samples would have to be drawn using EDTA as an anticoagulant to inhibit this activity.

C-type natriuretic peptide (CNP) a 22-amino acid peptide that is the primary active natriuretic peptide in the human brain; CNP is also considered to be an endothelium-derived relaxant factor, which acts in the same way as nitric oxide (NO) (Davidson et al., Circulation 93:1155-9, 1996). CNP is structurally related to Atrial natriuretic peptide (ANP) and B-type natriuretic peptide (BNP); however, while ANP and BNP are synthesized predominantly in the myocardium, CNP is synthesized in the vascular endothelium as a precursor (pro-CNP) (Prickett et al., Biochem. Biophys. Res. Commun. 286:513-7, 2001). CNP is thought to possess vasodilator effects on both arteries and veins and has been reported to act mainly on the vein by increasing the intracellular cGMP concentration in vascular smooth muscle cells.

Adrenomedullin (AM) is a 52-amino acid peptide which is produced in many tissues, including adrenal medulla, lung, kidney and heart (Yoshitomi et al., Clin. Sci. (Colch) 94:135-9, 1998). Intravenous administration of AM causes a long-lasting hypotensive effect, accompanied with an increase in the cardiac output in experimental animals. AM has been reported to enhance the stretch-induced release of ANP from the right atrium, but not to affect ventricular BNP expression. AM is synthesized as a precursor molecule (pro-AM). The N-terminal peptide processed from the AM precursor has also been reported to act as a hypotensive peptide (Kuwasako et al., Ann. Clin. Biochem. 36:622-8, 1999).

The endothelins are three related peptides (endothelin-1, endothelin-2, and endothelin-3) encoded by separate genes that are produced by vascular endothelium, each of which exhibit potent vasoconstricting activity. Endothelin-1 (ET-1) is a 21 amino acid residue peptide, synthesized as a 212 residue precursor (preproET-1), which contains a 17 residue signal sequence that is removed to provide a peptide known as big ET-1. This molecule is further processed by hydrolysis between trp21 and val22 by endothelin converting enzyme. Both big ET-1 and ET-1 exhibit biological activity; however the mature ET-1 form exhibits greater vasoconstricting activity (Brooks and Ergul, J. Mol. Endocrinol. 21:307-15, 1998). Similarly, endothelin-2 and endothelin-3 are also 21 amino acid residues in length, and are produced by hydrolysis of big endothelin-2 and big endothelin-3, respectively (Yap et al., Br. J. Pharmacol. 129:170-6, 2000; Lee et al., Blood 94:1440-50, 1999).

Urotensin 2 is a peptide having the sequence Ala-Gly-Thr-Ala-Asp-Cys-Phe-Trp-Lys-Tyr-Cys-Val, with a disulfide bridge between Cys6 and Cys 11. Human urotensin 2 (UTN) is synthesized in a prepro form. Processed urotensin 2 has potent vasoactive and cardiostimulatory effects, acting on the G protein-linked receptor GPR14.

Vasopressin (arginine vasopressin, AVP; antidiuretic hormone, ADH) is a peptide hormone released from the posterior pituitary. Its primary function in the body is to regulate extracellular fluid volume by affecting renal handling of water. There are several mechanisms regulating release of AVP. Hypovolemia, as occurs during hemorrhage, results in a decrease in atrial pressure. Specialized stretch receptors within the atrial walls and large veins (cardiopulmonary baroreceptors) entering the atria decrease their firing rate when there is a fall in atrial pressure. Afferent from these receptors synapse within the hypothalamus; atrial receptor firing normally inhibits the release of AVP by the posterior pituitary. With hypovolemia or decreased central venous pressure, the decreased firing of atrial stretch receptors leads to an increase in AVP release. Hypothalamic osmoreceptors sense extracellular osmolarity and stimulate AVP release when osmolarity rises, as occurs with dehydration. Finally, angiotensin II receptors located in a region of the hypothalamus regulate AVP release—an increase in angiotensin II simulates AVP release.

Heart Failure is also associated with what might be viewed as a paradoxical increase in AVP. Increased blood volume and atrial pressure associated with heart failure suggest that AVP secretion might be inhibited, but is is not. It may be that sympathetic and renin-angiotensin system activation in heart failure override the volume and low pressure cardiovascular receptors (as well as the osmoregulation of AVP) and cause an increase in AVP secretion. Nevertheless, this increase in AVP during heart failure may contribute to the increase in systemic vascular resistance as well as enhance renal retention of fluid.

AVP has two principle sites of action: kidney and blood vessels. The most important physiological action of AVP is that it increases water reabsorption by the kidneys by increasing water permeability in the collecting duct, thereby permitting the formation of a more concentrated urine. This is the antidiuretic effect of AVP. This hormone also constricts arterial blood vessels; however, the normal physiological concentrations of AVP are below its vasoactive range.

Calcitonin gene related peptide (CGRP) is a polypeptide of 37 amino acids that is a product of the calcitonin gene derived by alternative splicing of the precursor mRNA. The calcitonin gene (CALC-I) primary RNA transcript is processed into different mRNA segments by inclusion or exclusion of different exons as part of the primary transcript. Calcitonin-encoding mRNA is the main product of CALC-I transcription in C-cells of the thyroid, whereas CGRP-I mRNA (CGRP=calcitonin-gene-related peptide) is produced in nervous tissue of the central and peripheral nervous systems (FIG. 2.2.1) (9). In the third mRNA sequence, the calcitonin sequence is lost and alternatively the sequence of CGRP is encoded in the mRNA. CGRP is a markedly vasoactive peptide with vasodilatative properties. CGRP has no effect on calcium and phosphate metabolism and is synthesised predominantly in nerve cells related to smooth muscle cells of the blood vessels (149). ProCGRP, the precursor of CGRP, and PCT have partly identical N-terminal amino acid sequences.

Angiotensin II is an octapeptide hormone formed by renin action upon a circulating substrate, angiotensinogen, that undergoes proteolytic cleavage to from the decapeptide angiotensin I. Vascular endothelium, particularly in the lungs, has an enzyme, angiotensin converting enzyme (ACE), that cleaves off two amino acids to form the octapeptide, angiotensin II (AII).

All has several very important functions: Constricts resistance vessels (via All receptors) thereby increasing systemic vascular resistance and arterial pressure; Acts upon the adrenal cortex to release aldosterone, which in turn acts upon the kidneys to increase sodium and fluid retention; Stimulates the release of vasopressin (antidiuretic hormone, ADH) from the posterior pituitary which acts upon the kidneys to increase fluid retention; Stimulates thirst centers within the brain; Facilitates norepinephrine release from sympathetic nerve endings and inhibits norepinephrine re-uptake by nerve endings, thereby enhancing sympathetic adrenergic function; and Stimulates cardiac hypertrophy and vascular hypertrophy.

Glycated hemoglobin HbA1c measurement provides an assessment of the degree to which blood glucose has been elevated over an extended time period, and so has been related to the extent diabetes is controlled in a patient. Glucose binds slowly to hemoglobin A, forming the A1c subtype. The reverse reaction, or decomposition, proceeds relatively slowly, so any buildup persists for roughly 4 weeks. With normal blood glucose levels, glycated hemoglobin is expected to be 4.5% to 6.7%. As blood glucose concentration rise, however, more binding occurs. Poor blood sugar control over time is suggested when the glycated hemoglobin measure exceeds 8.0%.

(viii) Markers Related to Apoptosis

Caspase-3, also called CPP-32, YAMA, and apopain, is an interleukin-1 converting enzyme (ICE)-like intracellular cysteine proteinase that is activated during cellular apoptosis. Caspase-3 is present as an inactive 32 kDa precursor that is proteolytically activated during apoptosis induction into a heterodimer of 20 kDa and 11 kDa subunits (Fernandes-Alnemri, T. et al., J. Biol. Chem. 269:30761-30764, 1994). Its cellular substrates include poly(ADP-ribose) polymerase (PARP) and sterol regulatory element binding proteins (SREBPs) (Liu, X. et al., J. Biol. Chem. 271:13371-13376, 1996). The normal plasma concentration of caspase-3 is unknown. There are no published investigations into changes in the plasma concentration of caspase-3 associated with ACS. There are increasing amounts of evidence supporting the hypothesis of apoptosis induction in cardiac myocytes associated with ischemia and hypoxia (Saraste, A., Herz 24:189-195, 1999; Ohtsuka, T. et al., Coron. Artery Dis. 10:221-225, 1999; James, T. N., Coron. Artery Dis. 9:291-307, 1998; Bialik, S. et al., J. Clin. Invest. 100:1363-1372, 1997; Long, X. et al., J. Clin. Invest. 99:2635-2643, 1997). Elevations in the plasma caspase-3 concentration may be associated with any physiological event that involves apoptosis. There is evidence that suggests apoptosis is induced in skeletal muscle during and following exercise and in cerebral ischemia (Carraro, U. and Franceschi, C., Aging (Milano) 9:19-34, 1997; MacManus, J. P. et al., J. Cereb. Blood Flow Metab. 19:502-510, 1999).

Cathepsin D (E.C.3.4.23.5.) is a soluble lysosomal aspartic proteinase. It is synthesized in the endoplasmic reticulum as a preprocathepsin D. Having a mannose-6-phosphate tag, procathepsin D is recognized by a mannose-6-phosphate receptor. Upon entering into an acidic lysosome, the single-chain procathepsin D (52 KDa) is activated to cathepsin D and subsequently to a mature two-chain cathepsin D (31 and 14 KDa, respectively). The two mannose-6-phosphate receptors involved in the lysosomal targeting of procathepsin D are expressed both intracellularly and on the outer cell membrane. The glycosylation is believed to be crucial for normal intracellular trafficking. The fundamental role of cathepsin D is to degrade intracellular and internalized proteins. Cathepsin D has been suggested to take part in antigen processing and in enzymatic generation of peptide hormones. The tissue-specific function of cathepsin D seems to be connected to the processing of prolactin. Rat mammary glands use this enzyme for the formation of biologically active fragments of prolactin. Cathepsin D is functional in a wide variety of tissues during their remodeling or regression, and in apoptosis.

Brain α spectrin (also referred to as α fodrin) is a cytoskeletal protein of about 284 kDa that interacts with calmodulin in a calcium-dependent manner. Like erythroid spectrin, brain α spectrin forms oligomers (in particular dimers and tetramers). Brain a spectrin contains two EF-hand domains and 23 spectrin repeats. The caspase 3-mediated cleavage of a spectrin during apoptotic cell death may play an important role in altering membrane stability and the formation of apoptotic bodies.

Other Preferred Markers

The following table provides a list of additional preferred markers, associated with a disease or condition for which each marker can provide useful information for differential diagnosis. Various markers may be listed for more than one condition. As understood by the skilled artisan and described herein, markers may indicate different conditions when considered with additional markers in a panel; alternatively, markers may indicate different conditions when considered in the entire clinical context of the patient.

| Marker | Classification |
|---|---|
| Haptoglobin | Inflammatory |
| Hepcidin | Acute phase reactant |
| HSP-60 | Acute phase reactant |
| HSP-65 | Acute phase reactant |
| HSP-70 | Acute phase reactant |
| Myoglobin | Myocardial injury |
| PAPPA | Inflammatory |
| PECAM 1 | Acute phase reactant |
| Prostaglandin-D-Synthetase | Marker of ischemia |
| S100 | Myocardial injury |
| s-CD40 ligand* | Inflammatory |
| S-FAS ligand | Acute phase reactant |
| Troponin I and complexes | Myocardial injury |
| cardiotrophin 1 | Inflammatory |
| urotensin II | Blood pressure regulation |
| asymmetric dimethylarginine | Acute phase reactant |
| BNP | Blood pressure regulation |
| Fibrinogen | coagulation and hemostasis |
| ANP | Blood pressure regulation |
| CNP | Blood pressure regulation |
| Ubiquitin Fusion Degradation | |
| Protein I Homolog | Apoptosis |
| alpha 2 actin | Vascular tissue |
| basic calponin 1 | Vascular tissue |
| beta like 1 integrin | Vascular tissue |
| Calponin | Vascular tissue |
| CSRP2 | Vascular tissue |
| elastin | Vascular tissue |
| Fibrillin 1 | Vascular tissue |
| LTBP4 | Vascular tissue |
| smooth muscle myosin | Vascular tissue |
| transgelin | Vascular tissue |
| calcitonin gene related peptide | Blood pressure regulation |
| Carboxyterminal propeptide of | Marker of collagen synthesis |
| type I procollagen (PICP) | |
| Collagen carboxyterminal | Marker of collagen degradation |
| telopeptide (ICTP) | |
| Fibronectin | Inflammatory |
| MMP-11 | Acute phase reactant |
| MMP-3 | Acute phase reactant |
| MMP-9 | Acute phase reactant |
| arg-Vasopressin | Blood pressure regulation |
| aldosterone | Blood pressure regulation |
| angiotensin 1 | Blood pressure regulation |
| angiotensin 2 | Blood pressure regulation |
| angiotensin 3 | Blood pressure regulation |
| Antithrombin-III | coagulation and hemostasis |
| Bradykinin | Blood pressure regulation |
| calcitonin | Blood pressure regulation |
| Endothelin-2 | Blood pressure regulation |
| Endothelin-3 | Blood pressure regulation |
| Renin | Blood pressure regulation |
| Urodilatin | Blood pressure regulation |
| Defensin HBD 1 | Acute phase reactant |
| Defensin HBD 2 | Acute phase reactant |
| alpha enolase | Pulmonary tissue specific |
| LAMP 3 | Pulmonary tissue specific |
| LAMP3 | Pulmonary tissue specific |
| Lung Surfactant protein D | Pulmonary tissue specific |
| phospholipase D | Pulmonary tissue specific |
| PLA2G5 | Pulmonary tissue specific |
| SFTPC | Pulmonary tissue specific |
| D-dimer | coagulation and hemostasis |
| HMG | Inflammatory |
| IL-1 | Inflammatory |
| IL-8 | Inflammatory |
| IL-10* | Inflammatory |
| IL-11* | Inflammatory |
| IL-13* | Inflammatory |
| IL-18* | Inflammatory |
| IL-4* | Inflammatory |
| macrophage inhibitory factor | Inflammatory |
| s-acetyl Glutathione | apoptosis |
| Serum Amyloid A | Acute phase reactant |
| s-iL 18 receptor | pro and anti-Inflammatory modulator |
| S-iL-1 receptor | pro and anti-Inflammatory modulator |
| s-TNF P55 | Inflammatory and growth factor |
| s-TNF P75 | Inflammatory and growth factor |
| TGF-beta | Acute phase reactant |
| MMP-11 | Acute phase reactant |
| PAI-1 | coagulation and hemostasis |
| Procalcitonin | Blood pressure regulation |
| PROTEIN C | coagulation and hemostasis |
| TAFI | coagulation and hemostasis |
| CRP | Acute phase reactant |
| e-selectin | Acute phase reactant |
| 14-3-3 | Neural tissue injury |
| 4.1B | Neural tissue injury |
| adrenomedullin | Blood pressure regulation |
| APO E4-1 | Neural tissue injury |
| Atrophin 1 | Neural tissue injury |
| Beta NGF | Acute phase reactant |
| beta thromboglobulin | coagulation and hemostasis |
| BNP | Blood pressure regulation |
| brain Derived neurotrophic | Neural tissue injury |
| factor | |
| Brain Fatty acid binding protein | Neural tissue injury |
| brain tubulin | Neural tissue injury |
| CACNA1A | Neural tissue injury |
| Calbindin D | Neural tissue injury |
| Calbrain | Neural tissue injury |
| calcyphosine | Blood pressure regulation |
| Carbonic anhydrase XI | Neural tissue injury |
| Caspase 3 | apoptosis |
| Cathepsin D | apoptosis |
| CBLN1 | Neural tissue injury |
| CD44 | Inflammatory |
| Cerebellin 1 | Neural tissue injury |
| Chimerin 1 | Neural tissue injury |
| Chimerin 2 | Neural tissue injury |
| CHN1 | Neural tissue injury |
| CHN2 | Neural tissue injury |
| Ciliary neurotrophic factor | Neural tissue injury |
| CKBB | Neural tissue injury |
| CNP | Blood pressure regulation |
| CRHR1 | Neural tissue injury |
| C-tau | Neural tissue injury |
| cytochrome C | apoptosis |
| DRPLA | Neural tissue injury |
| EGF | Inflammatory and growth factors |
| Endothelin-1 | Blood pressure regulation |
| E-selectin | Acute phase reactant |
| Fibrinopeptide A | coagulation and hemostasis |
| Fibronectin | Inflammatory |
| GFAP | Neural tissue injury |
| Glutathione S Transferase | Acute phase reactant |
| GPM6B | Neural tissue injury |
| GPR7 | Neural tissue injury |
| GPR8 | Neural tissue injury |
| GRIN2C | Neural tissue injury |
| GRM7 | Neural tissue injury |
| HAPIP | Neural tissue injury |
| HIF 1 ALPHA | Acute phase reactant |
| HIP2 | Neural tissue injury |
| HSP-60 | Acute phase reactant |
| IL-10 | Inflammatory |
| IL-1-Beta | Inflammatory |
| IL-1ra | Inflammatory |
| IL-6 | Inflammatory |
| IL-8 | Inflammatory |
| I-NOS | Acute phase reactant |
| Insulin-like growth factor | Inflammatory |
| Intracellular adhesion molecule | Acute phase reactant |
| KCNK4 | Neural tissue injury |
| KCNK9 | Neural tissue injury |
| KCNQ5 | Neural tissue injury |
| Lactate dehydrogenase | Acute phase reactant |
| MAPK10 | Neural tissue injury |
| MCP-1 | Acute phase reactant |
| MDA-LDL | plaque rupture |
| MMP-3 | Acute phase reactant |
| MMP-9 | Acute phase reactant |
| myelin basic protein | Neural tissue injury |
| n-acetyl aspartate | Acute phase reactant |
| NCAM | Neural tissue injury |
| NDPKA | Neural tissue injury |
| Neural cell adhesion molecule | Neural tissue injury |
| NEUROD2 | Neural tissue injury |
| Neurofiliment L | Neural tissue injury |
| Neuroglobin | Neural tissue injury |
| neuromodulin | Neural tissue injury |
| Neuron specific enolase | Neural tissue injury |
| Neuropeptide Y | Neural tissue injury |
| Neurotensin | Neural tissue injury |
| Neurotrophin 1,2,3,4 | Neural tissue injury |
| NRG2 | Neural tissue injury |
| Osteoprotegerin | Inflammatory |
| PACE4 | Neural tissue injury |
| phosphoglycerate mutase | Neural tissue injury |
| PKC gamma | Neural tissue injury |
| Plasmin alpha 2 antiplasmin | coagulation and hemostasis |
| complex | |
| Platelet factor 4 | coagulation and hemostasis |
| Prostaglandin D-synthase | Acute phase reactant |
| Prostaglandin E2 | Acute phase reactant |
| proteolipid protein | Neural tissue injury |
| PTEN | Neural tissue injury |
| PTPRZ1 | Neural tissue injury |
| RANK ligand | Acute phase reactant |
| RGS9 | Neural tissue injury |
| RNA Binding protein Regulatory | |
| Subunit | Neural tissue injury |
| S-100b | Neural tissue injury |
| SCA7 | Neural tissue injury |
| secretagogin | Neural tissue injury |
| SLC1A3 | Neural tissue injury |
| SORL1 | Neural tissue injury |
| spectrin | apoptosis |
| SREB3 | Neural tissue injury |
| STAC | Neural tissue injury |
| STX1A | Neural tissue injury |
| STXBP1 | Neural tissue injury |
| Syntaxin | Neural tissue injury |
| Thrombin antithrombin III | coagulation and hemostasis |
| complex | |
| Thrombomodulin | coagulation and hemostasis |
| Thrombus Precursor Protein | coagulation and hemostasis |
| Tissue factor | coagulation and hemostasis |
| TNF Receptor Superfamily | Acute phase reactant |
| Member 1A | |
| Transforming growth factor beta | Inflammatory |
| transthyretin | Neural tissue injury |
| Tumor necrosis factor alpha | Acute phase reactant |
| Vascular cell adhesion molecule | Acute phase reactant |
| Vascular endothelial growth | Inflammatory |
| factor | |
| von Willebrand factor | coagulation and hemostasis |
| adenylate kinase-1 | Neural tissue injury |
| BDNF* | Neural tissue injury |
| CGRP | Blood pressure regulation |
| cystatin C | Acute phase reactant |
| neurokinin A | Neural tissue injury |
| substance P | Inflammatory |
| D Dimer | coagulation and hemostasis |
| Myeloperoxidase (MPO) | Inflammatory |
| Oxidized Low-Density | |
| Lipoproteins (OxLDL) | markers of atherosclerosis |

Exemplary Marker Panels for Distinguishing Systolic and Diastolic Heart Failure

Exemplary marker panels related to differentiating systolic and diastolic function comprise one or more markers selected from the group consisting of BNP, BNP related peptides, aldosterone, ANP, ANP related peptides, urodilatin, angiotensin 1, angiotensin 2, angiotensin 3, bradykinin, calcitonin, calcitonin gene related peptide, endothelin-2, endothelin-3, renin, urotensin 1, urotensin 2, antithrombin III, D-dimer, MMP-3, MMP-9, MMP-11, carboxy terminal propeptide of type I collagen (PICP), collagen carboxy terminal telopeptide (ICTP), fibrinogen, fibronectin, and vasopressin. Markers related to both systolic and diastolic dysfunction include BNP, ANP and ANP related markers. A preferred list of markers for differentiating systolic and diastolic heart failure include one or more markers selected from the group consisting of BNP, BNP related peptides, calcitonin gene related peptide, urotensin 2, endothelin 2, calcitonin and angiotensin 2. A particularly preferred list of markers for differentiating systolic and diastolic dysfunction include one or more markers selected from the group consisting of BNP, angiotensin 2, urotensin 2, and calcitonin gene related peptide.

Congestive heart failure is a heterogenous condition arising from two primary pathologies: left ventricular diastolic dysfunction and systolic dysfunction, which occur either alone or in combination. Gaasch, JAMA 271: 1276-80 (1994). As many as 40 percent of patients with clinical heart failure have diastolic dysfunction with normal systolic function. Soufer et al., Am. J. Cardiol. 55: 1032-6 (1984). Patient care decisions and prognosis hinge upon determination of the presence of one or both of these pathologies. Shamsham and Mitchell, Am. Fam. Physician 2000; 61:1319-28 (2000).

Recently, BNP has been reported as a useful marker in the diagnosis of congestive heart failure. Dao et al., J. Am. Coll. Cardiol. 37: 379-85 (2001). However, BNP levels alone are not able to distinguish diastolic dysfunction from systolic dysfunction. Krishnaswamy et al., Am. J. Med. 111: 274-79 (2001).

Exemplary Marker Panels for Distinguishing Aortic Dissection, Myocardial Ischemia and Myocardial Infarction

Exemplary marker panels related to differentiating aortic dissection, myocardial ischemia, and myocardial infarction comprise one or more markers selected from the group consisting of smooth muscle myosin and/or smooth muscle myosin heavy chain (both aortic dissection markers), BNP and/or BNP related peptides, one or more troponin forms (myocardial ischemia and infarction), and myoglobin (myocardial infarction or necrosis).

Exemplary Marker Panels for Distinguishing Atrial Fibrillation, Myocardial Infarction, and/or Congestive Heart Failure

Exemplary marker panels related to differentiating atrial fibrillation, myocardial infarction, and/or congestive heart failure comprise markers selected from the group consisting of ANP, ANP related peptides (atrial fibrillation), one or more troponin forms, myoglobin, BNP, and BNP related peptides.

Assay Measurement Strategies

Numerous methods and devices are well known to the skilled artisan for the detection and analysis of the markers of the instant invention. With regard to polypeptides or proteins in patient test samples, immunoassay devices and methods are often used. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety, including all tables, figures and claims. These devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, may be employed to determine the presence or amount of analytes without the need for a labeled molecule. See, e.g., U.S. Pat. Nos. 5,631,171; and 5,955,377, each of which is hereby incorporated by reference in its entirety, including all tables, figures and claims. One skilled in the art also recognizes that robotic instrumentation including but not limited to Beckman Access, Abbott AxSym, Roche ElecSys, Dade Behring Stratus systems are among the immunoassay analyzers that are capable of performing the immunoassays taught herein.

Preferably the markers are analyzed using an immunoassay, although other methods are well known to those skilled in the art (for example, the measurement of marker RNA levels). The presence or amount of a marker is generally determined using antibodies specific for each marker and detecting specific binding. Any suitable immunoassay may be utilized, for example, enzyme-linked immunoassays (ELISA), radioimmunoassays (RIAs), competitive binding assays, and the like. Specific immunological binding of the antibody to the marker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The use of immobilized antibodies specific for the markers is also contemplated by the present invention. The antibodies could be immobilized onto a variety of solid supports, such as magnetic or chromatographic matrix particles, the surface of an assay place (such as microtiter wells), pieces of a solid substrate material or membrane (such as plastic, nylon, paper), and the like. An assay strip could be prepared by coating the antibody or a plurality of antibodies in an array on solid support. This strip could then be dipped into the test sample and then processed quickly through washes and detection steps to generate a measurable signal, such as a colored spot.

The analysis of a plurality of markers may be carried out separately or simultaneously with one test sample. For separate or sequential assay of markers, suitable apparatuses include clinical laboratory analyzers such as the ElecSys (Roche), the AxSym (Abbott), the Access (Beckman), the ADVIA® CENTAUR® (Bayer) immunoassay systems, the NICHOLS ADVANTAGE® (Nichols Institute) immunoassay system, etc. Preferred apparatuses or protein chips perform simultaneous assays of a plurality of markers on a single surface. Particularly useful physical formats comprise surfaces having a plurality of discrete, adressable locations for the detection of a plurality of different analytes. Such formats include protein microarrays, or “protein chips” (see, e.g., Ng and Ilag, J. Cell Mol. Med. 6: 329-340 (2002)) and certain capillary devices (see, e.g., U.S. Pat. No. 6,019,944). In these embodiments, each discrete surface location may comprise antibodies to immobilize one or more analyte(s) (e.g., a marker) for detection at each location. Surfaces may alternatively comprise one or more discrete particles (e.g., microparticles or nanoparticles) immobilized at discrete locations of a surface, where the microparticles comprise antibodies to immobilize one analyte (e.g., a marker) for detection.

Several markers may be combined into one test for efficient processing of a multiple of samples. In addition, one skilled in the art would recognize the value of testing multiple samples (for example, at successive time points) from the same individual. Such testing of serial samples will allow the identification of changes in marker levels over time. Increases or decreases in marker levels, as well as the absence of change in marker levels, would provide useful information about the disease status that includes, but is not limited to identifying the approximate time from onset of the event, the presence and amount of salvagable tissue, the appropriateness of drug therapies, the effectiveness of various therapies as indicated by reperfusion or resolution of symptoms, differentiation of the various types of ACS, identification of the severity of the event, identification of the disease severity, and identification of the patient's outcome, including risk of future events.

A panel consisting of the markers referenced above may be constructed to provide relevant information related to differential diagnosis. Such a panel may be constucted using 1, 2, 3, 4, 5, 6, 7, 8, 9, 10, 15, 20, or more or individual markers. The analysis of a single marker or subsets of markers comprising a larger panel of markers could be carried out by one skilled in the art to optimize clinical sensitivity or specificity in various clinical settings. These include, but are not limited to ambulatory, urgent care, critical care, intensive care, monitoring unit, inpatient, outpatient, physician office, medical clinic, and health screening settings. Furthermore, one skilled in the art can use a single marker or a subset of markers comprising a larger panel of markers in combination with an adjustment of the diagnostic threshold in each of the aforementioned settings to optimize clinical sensitivity and specificity. The clinical sensitivity of an assay is defined as the percentage of those with the disease that the assay correctly predicts, and the specificity of an assay is defined as the percentage of those without the disease that the assay correctly predicts (Tietz Textbook of Clinical Chemistrys 2^{nd} edition, Carl Burtis and Edward Ashwood eds., W.B. Saunders and Company, p. 496).

The analysis of markers could be carried out in a variety of physical formats as well. For example, the use of microtiter plates or automation could be used to facilitate the processing of large numbers of test samples. Alternatively, single sample formats could be developed to facilitate immediate treatment and diagnosis in a timely fashion, for example, in ambulatory transport or emergency room settings.

In another embodiment, the present invention provides a kit for the analysis of markers. Such a kit preferably comprises devises and reagents for the analysis of at least one test sample and instructions for performing the assay. Optionally the kits may contain one or more means for using information obtained from immunoassays performed for a marker panel to rule in or out certain diagnoses.

Selecting a Treatment Regimen

Just as the potential causes of any particular nonspecific symptom may be a large and diverse set of conditions, the appropriate treatments for these potential causes may be equally large and diverse. However, once a diagnosis is obtained, the clinician can readily select a treatment regimen that is compatible with the diagnosis. Taking just some of the causes of dyspnea for example, initial treatment for pulmonary embolism is supportive, involving analgesics, oxygen, and potentially β-adrenergic stimulation. Thrombolytic therapy or embolectomy may be indicated. In contrast, treatment for systolic dysfunction in congestive heart failure can include therapeutic amounts of ACE inhibitors, digoxin, α-blockers, and diuretics. In particularly serious chronic heart failure, heart transplant may be indicated. The skilled artisan is aware of appropriate treatments for numerous diseases discussed in relation to the methods of diagnosis described herein. See, e.g., Merck Manual of Diagnosis and Therapy, 17^{th} Ed. Merck Research Laboratories, Whitehouse Station, N.J., 1999.

### EXAMPLES

The following examples serve to illustrate the present invention. These examples are in no way intended to limit the scope of the invention.

### Example 1

### Blood Sampling

Blood specimens were collected by trained study personnel using EDTA as the anticoagulant and centrifuged for greater than or equal to 10 minutes. The plasma component was transferred into a sterile cryovial and frozen at −20° C. or colder. Specimens from the following population of patients and normal healthy donors were collected (Table 1). Clinical histories were available for each of the patients to aid in the statistical analysis of the assay data.

### Example 2

### Biochemical Analyses

Markers were measured using standard immunoassay techniques. These techniques involved the use of antibodies to specifically bind the protein targets. A monoclonal antibody directed against a selected marker was biotinylated using N-hydroxysuccinimide biotin (NHS-biotin) at a ratio of about 5 NHS-biotin moieties per antibody. The antibody-biotin conjugate was then added to wells of a standard avidin 384 well microtiter plate, and antibody conjugate not bound to the plate was removed. This formed the “anti-marker” in the microtiter plate. Another monoclonal antibody directed against the same marker was conjugated to alkaline phosphatase using succinimidyl 4-[N-maleimidomethyl]-cyclohexane-1-carboxylate (SMCC) and N-succinimidyl 3-[2-pyridyldithio]propionate (SPDP) (Pierce, Rockford, Ill.).

Immunoassays were performed on a TECAN Genesis RSP 200/8 Workstation. Biotinylated antibodies were pipetted into microtiter plate wells previously coated with avidin and incubated for 60 min. The solution containing unbound antibody was removed, and the wells were washed with a wash buffer, consisting of 20 mM borate (pH 7.42) containing 150 mM NaCl, 0.1% sodium azide, and 0.02% Tween-20. The plasma samples (10 μL) were pipeted into the microtiter plate wells, and incubated for 60 min. The sample was then removed and the wells were washed with a wash buffer. The antibody-alkaline phosphatase conjugate was then added to the wells and incubated for an additional 60 min after which time, the antibody conjugate was removed and the wells were washed with a wash buffer. A substrate, (AttoPhos®, Promega, Madison, Wis.) was added to the wells, and the rate of formation of the fluorescent product was related to the concentration of the marker in the patient samples.

### Example 3

### Dyspnea Analysis

The following table compares levels of pulmonary surfactant protein D, D-dimer, BNP, total cardiac troponin I, and the ratio of BNP:D-dimer in individual patients presenting with clinical dyspnea and in normal subjects. Dyspnea patients were subdivided into patients receiving a clinical diagnosis of congestive heart failure, and those receiving a clinical diagnosis of pulmonary embolism. All units are ng/ml except BNP (pg/ml) and ratios.

Multi-Center CHF
Patients

| Patient ID | PSD | D-Dimer | BNP | Tnl | Ratio |
|---|---|---|---|---|---|
| 16 | 35.4 | 88 | 889 | 2.1 | 10.1 |
| 012 | 8.7 | 113 | 1228 | 2.2 | 10.9 |
| Moore 003 | 6.9 | 62 | 552 | 0.0 | 8.9 |
| 11 | 11.7 | 160 | 987 | 0.5 | 6.2 |
| 010 | 13.3 | 145 | 466 | 0.0 | 3.2 |
| 18 | 7.9 | 39 | 330 | 0.0 | 8.6 |
| 131-2 | 7.2 | 125 | 1031 | 0.0 | 8.3 |
| 125-1 | 3.7 | 49 | 314 | 0.0 | 6.4 |
| 115 | 8.1 | 203 | 185 | 0.0 | 0.9 |
| 128-1 | 7.5 | 141 | 228 | 0.0 | 1.6 |
| 143-1 | 5.1 | 169 | 402 | 0.0 | 2.4 |
| 134-1 | 1.9 | 142 | 251 | 0.0 | 1.8 |
| 138-1 | 2.4 | 40 | 521 | 0.0 | 13.0 |
| 157-1 | 4.1 | 107 | 231 | 0.0 | 2.2 |
| 176-1 | 2.6 | 70 | 234 | 0.0 | 3.4 |
| 175-1 | 4.6 | 154 | 498 | 0.0 | 3.2 |
| 22 | 6.7 | 36 | 650 | 0.0 | 18.3 |
| 21 | 3.9 | 149 | 453 | 0.0 | 3.0 |
| 23 | 11.5 | 147 | 1024 | 0.0 | 7.0 |
| 103-2 | 3.3 | 70 | 640 | 0.0 | 9.2 |
| 20 | 2.9 | 78 | 858 | 0.0 | 11.0 |
| 148-2 | 6.2 | 79 | 1614 | 0.0 | 20.4 |
| 173-2 | 2.7 | 68 | 236 | 0.0 | 3.5 |
| 166-1 | 5.5 | 53 | 681 | 0.0 | 12.9 |
| 178-1 | 4.3 | 89 | 250 | 0.0 | 2.8 |
| 183-2 | 9.3 | 109 | 1199 | 0.0 | 11.0 |
| 189-2 | 2.2 | 270 | 335 | 0.0 | 1.2 |
| 42 | 3.5 | 143 | 846 | 0.0 | 5.9 |
| 43 | 4.2 | 63 | 287 | 0.0 | 4.5 |
| 54 | 3.5 | 51 | 302 | 0.0 | 5.9 |
| 25 | 4.6 | 61 | 768 | 0.0 | 12.5 |
| 53 | 4.5 | 77 | 1813 | 0.0 | 23.5 |
| 59 | 20.8 | 77 | 288 | 0.0 | 3.7 |
| 55 | 2.4 | 53 | 237 | 0.0 | 4.5 |
| 158-1 | 2.3 | 53 | 1030 | 0.0 | 19.6 |
| Mean | 6.7 | 100.9 | 624.5 | 0.1 | 7.8 |
| Median | 4.6 | 79.3 | 498.0 | 0.0 | 6.2 |
| St. Dev. | 6.3 | 53.1 | 415.8 | 0.5 | 5.9 |

| | | | | | |
|---|---|---|---|---|---|
| Patient ID | PSD | D-Dimer | BNP | Tnl | Ratio |

Multi-Center Patients
with PE

| | | | | | |
|---|---|---|---|---|---|
| 81 | 4.9 | 145 | 314.7 | 0.0 | 2.2 |
| 110 | 4.3 | 87 | 24.3 | 0.0 | 0.3 |
| 112 | 6.9 | 105 | 15.9 | 0.0 | 0.2 |
| 119 | 10.1 | 104 | 175.7 | 0.0 | 1.7 |
| 142 | 7.0 | 106 | 6.2 | 0.0 | 0.1 |
| 196 | 8.2 | 127 | 5.0 | 0.1 | 0.0 |
| 801-2 | 5.2 | 113 | 19.7 | 0.0 | 0.2 |
| 377-2 | 1.4 | 97 | 57.2 | 0.0 | 0.6 |
| 008264 | 1.3 | 258 | 121.3 | 0.0 | 0.5 |
| 008557 | 17.5 | 126 | 51.3 | 0.0 | 0.4 |
| 010647 | 3.8 | 106 | 355.3 | 0.0 | 3.4 |
| 10640 | 0.7 | 43 | 9.2 | 0.0 | 0.2 |
| 7329 | 3.4 | 191 | 287.3 | 0.0 | 1.5 |
| 008605 | 6.0 | 82 | 733.5 | 0.0 | 9.0 |
| Mean | 5.8 | 120.7 | 155.5 | 0.0 | 1.4 |
| Median | 5.0 | 105.7 | 54.3 | 0.0 | 0.4 |
| St. Dev. | 4.3 | 51.7 | 207.6 | 0.0 | 2.4 |

Normal
Subjects

| | | | | | |
|---|---|---|---|---|---|
| 001511 | 5.1 | 90 | 20.4 | 0.0 | 0.2 |
| 001515 | 1.9 | 36 | 8.9 | 0.0 | 0.2 |
| 001520 | 1.0 | 61 | 6.5 | 0.0 | 0.1 |
| 001521 | 4.6 | 72 | 3.8 | 0.0 | 0.1 |
| 001524 | 2.3 | 69 | 11.1 | 0.0 | 0.2 |
| 001607 | 3.6 | 72 | 23.4 | 0.0 | 0.3 |
| 001610 | 1.1 | 52 | 18.3 | 0.0 | 0.4 |
| 001613 | 0.2 | 40 | 0.0 | 0.0 | 0.0 |
| 001616 | 2.6 | 28 | 0.0 | 0.0 | 0.0 |
| 001619 | 0.3 | 44 | 0.0 | 0.0 | 0.0 |
| 001622 | 1.4 | 25 | 0.0 | 0.0 | 0.0 |
| 001625 | 4.6 | 142 | 0.0 | 0.0 | 0.0 |
| 001628 | 1.6 | 40 | 0.0 | 0.0 | 0.0 |
| 001631 | 4.6 | 57 | 0.0 | 0.0 | 0.0 |
| 001634 | 7.2 | 60 | 6.6 | 0.0 | 0.1 |
| 001637 | 5.5 | 55 | 0.0 | 0.0 | 0.0 |
| 001640 | 0.0 | 260 | 19.1 | 0.0 | 0.1 |
| 001643 | 2.5 | 50 | 7.7 | 0.0 | 0.2 |
| 001646 | 0.0 | 56 | 4.7 | 0.0 | 0.1 |
| 002202 | 1.0 | 59 | 27.4 | 0.0 | 0.5 |
| 002205 | 1.7 | 39 | 23.4 | 0.0 | 0.6 |
| 002208 | 1.1 | 25 | 25.9 | 0.0 | 1.0 |
| 002211 | 0.9 | 55 | 45.9 | 0.0 | 0.8 |
| 002214 | 0.0 | 97 | 23.4 | 0.0 | 0.2 |
| 002217 | 2.8 | 117 | 15.3 | 0.0 | 0.1 |
| 002220 | 0.3 | 55 | 11.3 | 0.0 | 0.2 |
| 002223 | 2.5 | 47 | 8.1 | 0.0 | 0.2 |
| 002228 | 2.2 | 44 | 24.3 | 0.0 | 0.5 |
| 002229 | 2.6 | 61 | 11.2 | 0.0 | 0.2 |
| 002232 | 0.7 | 69 | 10.5 | 0.0 | 0.2 |
| 002235 | 0.0 | 54 | 4.0 | 0.0 | 0.1 |
| 002238 | 1.5 | 53 | 9.6 | 0.0 | 0.2 |
| 002241 | 7.5 | 16 | 10.8 | 0.0 | 0.7 |
| 002244 | 8.6 | 44 | 10.7 | 0.0 | 0.2 |
| 002247 | 3.7 | 68 | 33.1 | 0.0 | 0.5 |
| Mean | 2.5 | 63.3 | 12.2 | 0.0 | 0.2 |
| Median | 1.9 | 55.0 | 10.5 | 0.0 | 0.2 |
| St. Dev. | 2.3 | 42.3 | 11.1 | 0.0 | 0.3 |

These data indicate that the median D-dimer levels in the patients diagnosed with pulmonary embolism is higher than for the CHF patients, which is itself higher than normal subjects. Pulmonary surfactant protein D levels appears to be elevated over normals to nearly the same extent in both disease groups compared to normals. Using <82 μg/ml d-dimer as the rule-out cutoff for a diagnosis of pulmonary embolism would result in one false negative diagnosis, and would correctly rule out 18 of the 35 CHF patients and 30 of the 35 normals. For this patient population, using a d-dimer/BNP ratio of >3.4 as the rule-out cutoff would again result in one false negative diagnosis, but would correctly rule out 25 of the 35 CHF patients. The low cardiac troponin I level in all disease and normal subjects correctly rules out the occurrence of myocardial infarction in the entire test population. This example demonstrates that the differential diagnosis of causes of dyspnea can be accomplished through the measurement of d-dimer, BNP and cardiac troponin. Additionally, pulmonary embolism can be ruled in when BNP, d-dimer and pulmonary surfactant protein D levels are elevated above normal levels and troponin levels are normal. Pulmonary embolism can be ruled out when d dimer levels are in the normal range. When BNP levels are above normal, one can rule in congestive heart failure. When cardiac troponin levels are above normal, cardiac ischemia and necrosis can be ruled in.

### Example 4

### Identification of Diastolic Dysfunction

The following table compares levels of BNP, vasopressin, endothelin-2, calcitonin gene related peptide, urotensin 2, ANP, angiotensis 2, the ratios of BNP:CGRP, BNP:ANP, BNP:urotensin 2, and calcitonin in heart disease patients and normal subjects. The heart disease patients are subdivided according to the New York Heart Association classification of functional capacity and objective assessment. See, Nomenclature and Criteria for Diagnosis of Diseases of the Heart and Great Vessels. 9th ed. Boston, Mass: Little, Brown & Co; 1994, pp. 253-256. The classification is made as follows:

| Class | Functional Capacity | Objective Assessment |
|---|---|---|
| NYHA1 | Patients with cardiac disease but | No objective evidence |
| | without resulting limitation of physical | of cardiovascular |
| | activity. Ordinary physical activity does | disease. |
| | not cause undue fatigue, palpitation, | |
| | dyspnea, or anginal pain. | |
| NYHA2 | Patients with cardiac disease resulting in | Objective evidence |
| | slight limitation of physical activity. | of minimal |
| | They are comfortable at rest. Ordinary | cardiovascular disease. |
| | physical activity results in fatigue, | |
| | palpitation, dyspnea, or anginal pain. | |
| NYHA3 | Patients with cardiac disease resulting | Objective evidence of |
| | in marked limitation of physical | moderately severe |
| | activity. They are comfortable at rest. | cardiovascular disease. |
| | Less than ordinary activity causes | |
| | fatigue, palpitation, dyspnea, or anginal | |
| | pain. | |
| NYHA4 | Patients with cardiac disease resulting | Objective evidence of |
| | in inability to carry on any physical | severe cardiovascular |
| | activity without discomfort. Symptoms | disease. |
| | of heart failure or the anginal syndrome | |
| | may be present even at rest. If any | |
| | physical activity is undertaken, | |
| | discomfort is increased. | |

DD indicates patients having a clinical diagnosis of diastolic dysfunction, and exhibit an ejection fraction of >50%. Low ejection fraction (EF) patients are those exhibiting an ejection fraction of <50%, and are NYHA4 class patients considered to exhibit systolic, rather than diastolic, dysfunction. All units are ng/ml except BNP (pg/ml) and ratios, and N is the number of subjects in each group.

TABLE 4

| | | | | | BNP/ | | | BNP/ | | BNP/ | Angiotensin | |
|---|---|---|---|---|---|---|---|---|---|---|---|---|
| | BNP | Vasopressin | Endothelin 2 | CGRP | CGRP | Calcitonin | Urotensin 2 | U2 | ANP | ANP | 2 | N |
| Normal | 0 | 0.98 | 3.60 | 0.94 | 0 | 0.14 | 14.6 | 0 | 1.84 | 0 | 0.09 | 20 |
| DD NYHA1 | 142 | 1.13 | 4.63 | 1.06 | 247 | 0.20 | 18.9 | 11 | 2.11 | 162 | 0.09 | 2 |
| DD NYHA2 | 152 | 0.89 | 3.70 | 0.77 | 289 | 0.19 | 18.4 | 9 | 1.48 | 113 | 0.08 | 6 |
| DD NYHA3 | 325 | 0.84 | 3.72 | 1.02 | 309 | 0.16 | 18.8 | 16 | 1.95 | 149 | 0.09 | 6 |
| DD NYHA4 | 600 | 0.99 | 4.38 | 0.66 | 853 | 0.17 | 19.8 | 30 | 1.07 | 297 | 0.06 | 6 |
| DD All | 262 | 0.89 | 3.91 | 0.77 | 366 | 0.19 | 19.0 | 14 | 1.50 | 177 | 0.08 | |
| Low EF | 839 | 1.10 | 4.38 | 0.97 | 957 | 0.19 | 28.0 | 41 | 2.17 | 413 | 0.07 | 20 |

These data indicate that Urotensin-2 and ANP can distinguish diastolic dysfunction from systolic dysfunction. In both cases, the levels are higher in systolic dysfunction than in diastolic dysfunction. Moreover, with the addition of BNP, the the ability to discriminate diastolic dysfunction from systolic dysfunction is enhanced, as elevation of both BNP and ANP appears to be indicative of systolic dysfunction while elevation of BNP with ANP at or below normal levels appears to be indicative of diastolic dysfunction. Urotensin 2 shows a similar pattern. CGRP contributes to the ability to distinguish diastolic from systolic dysfunction when expressed as a ratio with BNP where the ratio is greater in cases of systolic dysfunction relative to diastolic dysfunction.

### Example 5

### Marker Panels for Caridac Differential Diagnosis

Exemplary marker panels were selected initially comprising a marker related to blood pressure regulation and a plurality of markers related to myocardial injury in order to develop a panel for diagnosing and/or distinguishing congestive heart failure, acute coronary syndromes, and myocardial infarction, and for guiding therapy in response to the results of the assay. For this purpose, BNP, cardiac troponin I (free and complexed), creatine kinase-MB, and myoglobin were selected. Threshold levels for comparison of measured marker concentrations were established in this example using the upper end of normal values for CKMB (4.3 ng/mL), myoglobin (107 ng/mL) and troponin 1 (0.4 ng/mL). Elevation and/or Temporal changes in these three markers, coupled with chest pain for a period of at least 20 minutes is highly indicative of myocardial infarction. In addition, BNP concentrations in excess of 80 pg/mL BNP can provide additional risk stratification in these patients, as this level of BNP is related to increased rates of death, myocardial infarction, and congestive heart failure in comprarison to patients having a BNP level below this threshold. Moreover, even in subjects experiencing no clinical symptoms of disease, a BNP level in excess of 100 pg/mL is associated with a substantially higher incidence of congestive heart failure. Thus, this multimarker strategy can provide substantially more clinically relevant information than can individual markers.

The addition of other markers to the multimarker panel can provide additional clinical information for both risk stratification and differential diagnosis. For example, D-dimer may be added to the panel as a marker of coagulation and hemostasis. As discussed above, the addition of D-dimer can permit the differentiation of pulmonary embolism and/or deep venous thrombosis from myocardial infarction and congestive heart failure, despite the fact that the subjects may present to the clinician with substantially similar symptoms. In this case, a threshold level of about about 1 μg/mL may be established. In addition, or in the alternative, to D-dimer, C-reactive protein, a relatively nonspecific indicator of inflammation, can provide additional risk stratification to the panel.

As the number of markers in a panel increases, the determination of a single panel response and its correlation to various disease states by the methods described herein can be advantageous. An example of such a panel may include rdiac troponin I (free and complexed), creatine kinase-MB, and myoglobin, where none of the markers are compared to a predetermined threshold. Instead, of values for each marker within a window are assigned a relative index (e.g., a value between 0 and 1). This window can be defined by a midpoint and a window width (e.g., for troponin I, a midpoint of 1.16 ng/mL with a window extending from 0.25 ng/mL to 2.06 ng/mL can be used; troponin I concentrations less than 0.25 are assigned an index value of 0, concentrations greater than 2.06 are assigned an index value of 1, and concentrations in the window are assigned a relative value between 0 and 1, using a linear interpolation across the window. This is repeated for each of CK-MB and myoglobin, with different midpoints and window widths determined according to the methods described herein. Finally, each marker may be assigned a weighting factor, by which the index value is multiplied. The three index values are added, and used to determine a panel response, which may be correlated to diagnosis (e.g., of acute myocardial infarction). Additional markers may be included, which may include single concentrations of markers, or may include relative changes in markers over time (e.g., an increase in troponin I, CK-MB and/or myoglobin concentration over 60-240 minutes may act as a marker in the panel).

In the case of acute myocardial infarction, panels, window values, and weighting factors are selected that, using a panel response value, preferably provide a sensitivity of at least 80% at greater than 90% specificity.

### Example 5

### Marker Panels for Cerebrovascular Differential Diagnosis

In the case of cerebrovascular differential diagnosis, marker panels were selected comprising a marker related to blood pressure regulation and a plurality of markers related to neural tissue injury in order to develop a panel for diagnosing and/or distinguishing stroke from patients referred to herein as “stroke mimics.” Additional classes of markers tested to increase marker panel response include markers of apoptosis, markers of inflammation, and/or acute phase reactants. A final exemplary panel was identified that provided a sensitivity of at least 80% at greater than 90% specificity. The details of this panel are described in the following table. For each marker, window values are defined as a window midpoint (“Midpoint”), and a window width (“Window”), which when multiplied by the midpoint provide the +/−range for the window width. A positive window width and weighting indicate a positive correlation of the marker with a diagnosis of stroke (that is, the marker level is increased in the stroke group relative to the mimic group); while a negative window width and weighting indicate a negative correlation of the marker with a diagnosis of stroke. Again, none of the markers are compared to a predetermined threshold. Instead, a panel response value is obtained as described herein.

| | | |
|---|---|---|
| Midpoint | Window | Weight |

NCAM

| | | |
|---|---|---|
| 81.610 ng/mL | −0.535 | −0.203 |

Caspase-3

| | | |
|---|---|---|
| 10.528 ng/mL | 0.422 | 0.172 |

IL-8

| | | |
|---|---|---|
| 54.727 pg/mL | 0.637 | 0.078 |

CK-BB

| | | |
|---|---|---|
| 3.232 ng/mL | 0.559 | 0.133 |

CRP

| | | |
|---|---|---|
| 90.283 μg/mL | 0.613 | 0.163 |

S-100b

| | | |
|---|---|---|
| 214.559 pg/mL | 0.409 | 0.112 |

BNP

| | | |
|---|---|---|
| 302.561 pg/mL | 0.465 | 0.092 |

MMP-9

| | | |
|---|---|---|
| 473.153 ng/mL | 0.590 | 0.046 |

| Analyte panel size | 8 | | |
|---|---|---|---|
| Stroke Type | All stroke | Ischemic stroke | Hemorrhagic |
| | | | stroke |
| Total Controls | 49 | 49 | 49 |
| Disease group | 48 | 41 | 7 |
| ROC Area | 0.956 | 0.969 | 0.878 |
| Sens @ 92.5% Spec | 93.8% | 95.1% | 85.7% |
| Spec @ 92.5% Sens | 95.9% | 95.9% | 22.4% |
| Runs (N) | 100 | 100 | 100 |
| Ave ROC Area | 0.927 | 0.935 | 0.881 |
| SD | 0.018 | 0.024 | 0.039 |
| Ave Sens @ 92.5% Spec | 85.6% | 86.8% | 78.7% |
| SD | 6.50% | 7.20% | 9.60% |
| Ave Spec @ 92.5% Sens | 83.2% | 84.4% | 39.8% |
| SD | 8.10% | 8.90% | 27.80% |

In addition, panels were assessed for the ability to identify severity of neurologic deficit in stroke patients. In these panels, controls were subjects exhibiting an NIH stroke scale (“NIHSS”) score of <5, while the disease subjects exhibited an NIHSS score of 5. As shown in the following table, simply changing the window parameters and weighting, while using the same eight markers described above, can provide important information about the severity of neurologic deficit.

| Panel 1 | | | Panel 2 | | |
|---|---|---|---|---|---|
| | Win- | | | Win- | |
| Midpoint | dow | Weight | Midpoint | dow | Weight |
| CRP | | | CRP | | |
| 15.229 μg/mL | 0.577 | 0.159 | 21.973 μg/mL | 0.573 | 0.180 |
| BNP | | | BNP | | |
| 254.365 pg/mL | 0.408 | 0.121 | 240.978 pg/mL | 0.438 | 0.118 |
| CK-BB | | | IL-8 | | |
| 0.370 ng/mL | 0.280 | 0.074 | 41.354 pg/mL | 0.519 | 0.149 |
| IL-8 | | | MMP-9 | | |
| 36.565 pg/mL | 0.382 | 0.136 | 438.778 ng/mL | −0.253 | −0.117 |
| Caspase-3 | | | CK-BB | | |
| 6.552 ng/mL | 0.482 | 0.103 | 0.847 ng/mL | 0.347 | 0.060 |
| MMP-9 | | | Caspase-3 | | |
| 445.855 ng/mL | −0.327 | −0.122 | 6.497 ng/mL | 0.574 | 0.095 |
| S-100b | | | S-100b | | |
| 5.248 pg/mL | 0.009 | −0.023 | 144.698 pg/mL | 0.244 | 0.042 |
| NCAM | | | NCAM | | |
| 66.384 ng/mL | −0.264 | −0.100 | 58.061 ng/mL | −0.202 | −0.069 |

| | Panel 1 | Panel 2 |
|---|---|---|
| Total Controls | 66 | 66 |
| Disease group | 24 | 24 |
| ROC Area | 0.966 | 0.963 |
| Sens @ 92.5% Spec | 100.0% | 100.0% |
| Spec @ 92.5% Sens | 95.5% | 95.5% |
| Runs (N) | 100 | 100 |
| Ave ROC Area | 0.928 | 0.929 |
| SD | 0.021 | 0.018 |
| Ave Sens @ 92.5% | 79.6% | 80.2% |
| Spec | | |
| SD | 12.70% | 13.30% |
| Ave Spec @ 92.5% | 86.9% | 87.7% |
| Sens | | |
| SD | 6.80% | 6.10% |

Interestingly, MMP-9 shows a negative correlation with neurologic deficit, indicating that, while MMP-9 is increased in stroke patients relative to mimics, MMP-9 is actually decreased in the case of stroke patients exhibiting an increased neurologic deficit, relative to subjects with less severe neurologic deficit. High MMP-9 may be indicative of increased revascularization, and therefore may be a marker of positive prognosis in stroke patients. In addition, thrombolytic treatment may be less advantageous in stroke patients with high MMP-9, as revascularization is providing additional perfusion of the lesion. Such panels may provide prognostic information in diseases and procedures that are associated with a risk of neurologic deficit. Such procedures include carotid endarterectomy, hypothermic circulatory arrest, aortic valve replacement, mitral valve replacement, coronary artery surgery, endograft repair of aortic aneurism, coronary artery bypass graft surgery, laryngeal mask insertion, and repair of congenital heart defects.

Additional panels may be provided that utilize fewer markers, with moderate loss of sensitivity and specificity, as shown in the following tables:

| Panel 3 | | | Panel 4 | | | Panel 5 | | | Panel 6 | | |
|---|---|---|---|---|---|---|---|---|---|---|---|
| Midpoint | Window | Weight | Midpoint | Window | Weight | Midpoint | Window | Weight | Midpoint | Window | Weight |
| CRP | | | CRP | | | CRP | | | CRP | | |
| 20.232 | 0.572 | 0.195 | 31.980 | 0.517 | 0.213 | 26.976 | 0.580 | 0.258 | 28.064 | 0.515 | 0.292 |
| BNP | | | BNP | | | BNP | | | BNP | | |
| 257.043 | 0.390 | 0.143 | 235.623 | 0.284 | 0.160 | 214.202 | 0.294 | 0.199 | 262.398 | 0.277 | 0.260 |
| MMP-9 | | | CK-BB | | | CK-BB | | | Caspase-3 | | |
| −545.945 | −0.335 | −0.169 | 0.567 | 0.474 | 0.086 | 0.832 | 0.483 | 0.126 | 7.753 | 0.592 | 0.186 |
| CK-BB | | | Caspase-3 | | | Caspase-3 | | | CK-BB | | |
| 0.990 | 0.441 | 0.083 | 6.825 | 0.522 | 0.120 | 7.699 | 0.654 | 0.177 | 0.166 | 0.373 | 0.100 |
| Caspase-3 | | | MMP-9 | | | MMP-9 | | | | | |
| 7.508 | 0.595 | 0.106 | −464.054 | −0.362 | −0.156 | −518.648 | −0.443 | −0.188 | | | |
| IL-8 | | | IL-8 | | | | | | | | |
| 37.149 | 0.537 | 0.145 | 43.924 | 0.463 | 0.174 | | | | | | |
| S-100b | | | | | | | | | | | |
| 182.184 | 0.442 | 0.079 | | | | | | | | | |
| | 81 | | | 86 | | | 86 | | | 86 | |
| | 26 | | | 27 | | | 27 | | | 27 | |
| | 0.941 | | | 0.947 | | | 0.922 | | | 0.900 | |
| | 100.0% | | | 96.3% | | | 85.2% | | | 66.7% | |
| | 92.6% | | | 93.0% | | | 87.2% | | | 84.9% | |
| | 100 | | | 100 | | | 100 | | | 100 | |
| | 0.913 | | | 0.910 | | | 0.895 | | | 0.878 | |
| | 0.021 | | | 0.019 | | | 0.014 | | | 0.016 | |
| | 74.8% | | | 73.4% | | | 65.3% | | | 61.0% | |
| | 12.60% | | | 10.50% | | | 7.90% | | | 6.00% | |
| | 83.6% | | | 83.7% | | | 80.9% | | | 76.1% | |
| | 6.80% | | | 5.50% | | | 5.10% | | | 7.40% | |

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

We claim:

1. A method of analyzing a subject sample for a plurality of subject-derived markers selected to distinguish amongst a plurality of cardiovascular disorders, comprising:
assaying said sample for the presence or amount of one or more subject-derived markers related to blood pressure regulation, and for the presence or amount of one or more subject-derived markers related to myocardial injury, and
characterizing said subject's risk of having developed or of developing each of said plurality of cardiovascular disorders based upon the presence or amount of said markers, wherein the amount of at least one of said one or more subject-derived markers is not compared to a predetermined threshold amount.

2. A method according to claim 1, wherein said characterization step is performed without comparing the amount of any of said markers to a predetermined threshold amount.

3. A method according to claim 1, wherein said subject-derived marker(s) related to blood pressure regulation are selected from the group consisting of B-type natriuretic peptide, a marker related to B-type natriuretic peptide, C-type natriuretic factor, urotensin II, arginine vasopressin, aldosterone, angiotensin I, angiotensin II, angiotensin III, bradykinin, calcitonin, procalcitonin, calcitonin gene related peptide, adrenomedullin, calcyphosine, endothelin-2, endothelin-3, rennin, A-type natriuretic peptide, and urodilatin, and wherein said subject-derived marker(s) related to myocardial injury are selected from the group consisting of free cardiac troponin I, free cardiac troponin T, cardiac troponin I in a complex comprising one or both of troponin T and troponin C, cardiac troponin T in a complex comprising one or both of troponin I and troponin C, free and complexed cardiac troponin I, free and complexed cardiac troponin T, creatine kinase-MB, myoglobin, glycogen phosphorylase-BB, annexin B, P-enolase, heart-type fatty acid binding protein, and S-100ao.

4. A method according to claim 3, wherein said method comprises assaying said sample for the presence or amount of B-type natriuretic peptide or a marker related to B-type natriuretic peptide, creatine kinase-MB, total cardiac troponin I, and myoglobin.

5. A method according to claim 1, wherein said method further comprises assaying said sample for the presence or amount of one or more subject-derived markers related to inflammation.

6. A method according to claim 5, wherein said characterization step is performed without comparing the amount of any of said marker(s) related to inflammation to a predetermined threshold amount.

7. A method according to claim 5, wherein said marker(s) related to inflammation are selected from the group consisting of C-reactive protein, an interleukin, interleukin-1 receptor agonist, CD54, CD106, monocyte chemotactic protein-1, caspase-3, lipocalin-type prostaglandin D synthase, mast cell tryptase, eosinophil cationic protein, KL-6, haptoglobin, tumor necrosis factor α, tumor necrosis factor β, fibronectin, and vascular endothelial growth factor.

8. A method according to claim 7, wherein said method comprises assaying said sample for the presence or amount of B-type natriuretic peptide or a marker related to B-type natriuretic peptide, creatine kinase-MB, total cardiac troponin I, myoglobin, and C-reactive protein.

9. A method according to claim 1, wherein said method further comprises assaying said sample for the presence or amount of one or more subject-derived markers related to coagulation and hemostasis.

10. A method according to claim 9, wherein said characterization step is performed without comparing the amount of any of said marker(s) related to coagulation and hemostasis to a predetermined threshold amount.

11. A method according to claim 9, wherein said subject-derived marker(s) related to coagulation and hemostasis are selected from the group consisting of plasmin, fibrinogen, D-dimer, β-thromboglobulin, platelet factor 4, fibrinopeptide A, platelet-derived growth factor, prothrombin fragment 1+2, plasmin-α2-antiplasmin complex, thrombin-antithrombin III complex, P-selectin, thrombin, von Willebrand factor, tissue factor, and thrombus precursor protein.

12. A method according to claim 11, wherein said method comprises assaying said sample for the presence or amount of B-type natriuretic peptide or a marker related to B-type natriuretic peptide, D-dimer, creatine kinase-MB, total cardiac troponin I, and myoglobin.

13. A method according to claim 5, wherein said method further comprises assaying said sample for the presence or amount of a subject-derived marker related to coagulation and hemostasis.

14. A method according to claim 13, wherein said method comprises assaying said sample for the presence or amount of B-type natriuretic peptide or a marker related to B-type natriuretic peptide, D-dimer, creatine kinase-MB, total cardiac troponin I, myoglobin, and C-reactive protein.

15. A method according to claim 1, wherein said subject sample is selected from the group consisting of a blood sample, a serum sample, and a plasma sample.

16. A method according to claim 1, wherein said plurality of cardiovascular disorders are selected from the group consisting of myocardial infarction, congestive heart failure, acute coronary syndrome, unstable angina, and pulmonary embolism.

17. A method according to claim 1, wherein said correlating step comprises comparing at least one marker amount to a predetermined threshold level.

18. A test device for performing the method of claim 1, comprising:
a test surface comprising a plurality of discrete addressable locations corresponding to said plurality of subject-derived markers, each said location comprising an antibody immobilized at said location selected to bind for detection one of said plurality of subject-derived markers.

19. A method of analyzing a subject sample for a plurality of subject-derived markers selected to distinguish amongst a plurality of cerebrovascular disorders, comprising:
assaying said sample for the presence or amount of one or more subject-derived markers related to blood pressure regulation, and for the presence or amount of one or more subject-derived markers related to neural tissue injury, and
characterizing said subject's risk of having developed or of developing each of said plurality of cerebrovascular disorders based upon the presence or amount of said markers, wherein the amount of one or more of said markers is not compared to a predetermined threshold amount.

20. A method according to claim 19, wherein said characterization step is performed without comparing the amount of any of said markers to a predetermined threshold amount.

21. A method according to claim 19, wherein said subject-derived marker(s) related to blood pressure regulation are selected from the group consisting of B-type natriuretic peptide, a marker related to B-type natriuretic peptide, C-type natriuretic factor, urotensin II, arginine vasopressin, aldosterone, angiotensin I, angiotensin II, angiotensin III, bradykinin, calcitonin, procalcitonin, calcitonin gene related peptide, adrenomedullin, calcyphosine, endothelin-2, endothelin-3, rennin, A-type natriuretic peptide, and urodilatin, and wherein said subject-derived marker(s) related to neural tissue injury are selected from the group consisting of precerebellin 1, cerebillin 1, cerebellin 3, chimerin 1, chimerin 2, calbrain, calbindin D, brain tubulin, brain fatty acid binding protein (“B-FABP”), brain derived neurotrophic factor (“BDNF”), carbonic anhydrase XI, CACNA1A calcium channel gene, nerve growth factor β, atrophin 1, apolipoprotein E4-1, protein 4.1B, 14-3-3 protein, ciliary neurotrophic factor, creatine kinase-BB, C-tau, glial fibrillary acidic protein (“GFAP”), neural cell adhesion molecule (“NCAM”), neuron specific enolase, S-100b, prostaglandin D synthase, neurokinin A, neurotensin, and secretagogin.

22. A method according to claim 19, wherein said method further comprises assaying said sample for the presence or amount of one or more subject-derived markers related to inflammation.

23. A method according to claim 22, wherein said characterization step is performed without comparing the amount of any of said marker(s) related to inflammation to a predetermined threshold amount.

24. A method according to claim 19, wherein said method further comprises assaying said sample for the presence or amount of one or more subject-derived markers related to coagulation and hemostasis.

25. A method according to claim 24, wherein said characterization step is performed without comparing the amount of any of said marker(s) related to coagulation and hemostasis to a predetermined threshold amount.

26. A method according to claim 19, wherein said method further comprises assaying said sample for the presence or amount of one or more subject-derived markers related to apoptosis.

27. A method according to claim 26, wherein said subject-derived marker(s) related to apoptosis are selected from the group consisting of spectrin, cathepsin D, caspase 3, s-acetyl glutathione, and ubiquitin fusion degradation protein 1 homolog.

28. A method according to claim 27, wherein said characterization step is performed without comparing the amount of any of said marker(s) related to apoptosis to a predetermined threshold amount.

29. A method according to claim 19, wherein said method further comprises assaying said sample for the presence or amount of one or more subject-derived acute phase markers.

30. A method according to claim 29, wherein said subject-derived acute phase marker(s) are selected from the group consisting of hepcidin, HSP-60, HSP-65, HSP-70, S-FAS ligand, asymmetric dimethylarginine, matrix metalloproteins 11, 3, and 9, defensin HBD 1, defensin HBD 2, serum amyloid A, oxidized LDL, insulin like growth factor, transforming growth factor β, e-selectin, glutathione-S-transferase, hypoxia-inducible factor-1α, inducible nitric oxide synthase, intracellular adhesion molecule, lactate dehydrogenase, monocyte chemoattractant peptide-1, n-acetyl aspartate, prostaglandin E2, receptor activator of nuclear factor ligand, TNF receptor superfamily member 1A, TNFα, vascular cell adhesion molecule, and cystatin C.

31. A method according to claim 27, wherein said characterization step is performed without comparing the amount of any of said acute phase marker(s) to a predetermined threshold amount.

32. A method according to claim 19, wherein said method comprises assaying said sample for the presence or amount of B-type natriuretic peptide or a marker related to B-type natriuretic peptide, caspase-3, interleukin-8, creatine kinase-BB, C-reactive protein, S-100b, matrix metalloprotein-9, and neural cell adhesion molecule.

33. A method according to claim 19, wherein said plurality of cerebrovascular disorders are selected from the group consisting of ischemic stroke, hemorrhagic stroke, transient ischemic attack, and subarachnoid hemorrhage.

34. A method according to claim 19, wherein said subject sample is selected from the group consisting of a blood sample, a serum sample, and a plasma sample.

35. A test device for performing the method of claim 19, comprising:
a test surface comprising a plurality of discrete addressable locations corresponding to said plurality of subject-derived markers, each said location comprising an antibody immobilized at said location selected to bind for detection one of said plurality of subject-derived markers.

36. A method of analyzing a subject sample for a plurality of subject-derived markers selected to identify subjects suffering from myocardial infarction, comprising:
assaying said sample for the presence or amount of a plurality of subject-derived marker(s) related to myocardial injury selected from the group consisting of free cardiac troponin I, free cardiac troponin T, cardiac troponin I in a complex comprising one or both of troponin T and troponin C, cardiac troponin T in a complex comprising one or both of troponin I and troponin C, free and complexed cardiac troponin I, free and complexed cardiac troponin T, creatine kinase-MB, myoglobin, glycogen phosphorylase-BB, annexin B, β-enolase, heart-type fatty acid binding protein, and S-100ao, and
characterizing said subject's risk of having suffered a myocardial infarction based upon the presence or amount of said markers, wherein the amount of each of said markers is not compared to a predetermined threshold amount.
